(19) United States
(12) Patent Application Publication
(10) Pub. No.: US 2026/0085034 A1
(43) Pub. Date: Mar. 26, 2026

(54) N-(BENZHYDRYL)CYCLOALKYLCARBOXAMIDE DERIVATIVES AS INHIBITORS OF GLYCOGEN SYNTHASE 1 (GYS1) AND METHODS OF USE THEREOF

(71) Applicant: Maze Therapeutics, Inc., South San Francisco, CA (US)

(72) Inventors: David John MORGANS, JR., Los Altos, CA (US); Kevin MELLEM, Redwood City, CA (US); Hannah L. POWERS, San Diego, CA (US); Patrick Sang Tae LEE, Walnut Creek, CA (US); Walter WON, San Diego, CA (US); Christopher Joseph SINZ, Walnut Creek, CA (US)

(21) Appl. No.: 19/111,738

(22) PCT Filed: Sep. 13, 2023
(86) PCT No.: PCT/US2023/074110

§ 371 Date: Mar. 13, 2025

Related U.S. Application Data
(60) Provisional application No. 63/406,684, filed on Sep. 14, 2022.

(51) Int. Cl. C07C 233/62; A61K 31/166; A61K 31/397; A61K 31/404; A61K 31/416; A61K 31/4166; A61K 31/421; A61K 31/4439; A61K 31/445; C07C 233/63; C07C 255/46; C07D 205/04; C07D 209/34; C07D 213/40; C07D 231/12; C07D 233/74; C07D 263/58; C07D 401/10

(52) U.S. Cl. CPC C07C 233/62; A61K 31/166; A61K 31/397; A61K 31/404; A61K 31/416; A61K 31/4166; A61K 31/421; A61K 31/4439; A61K 31/445; C07C 233/63; C07C 255/46; C07D 205/04; C07D 209/34; C07D 213/40; C07D 231/12; C07D 233/74; C07D 263/58; C07D 401/10

(57) ABSTRACT

Provided herein are compounds of formula (I) or a stereoisomer or tautomer thereof, or a pharmaceutically acceptable salt of any of the foregoing, wherein m, n, R¹, R², R³, R⁴, Y¹, Y², X¹, X², L, and Q¹ are as defined elsewhere herein. Also provided herein CN are methods of preparing compounds of formula (I). Also provided herein are methods of inhibiting GYSI and methods of treating a GYS1-mediated disease, disorder, or condition in an individual in need thereof.

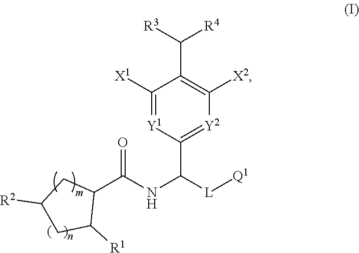

### CROSS REFERENCE TO RELATED APPLICATIONS

This application claims priority to U.S. Provisional Application No. 63/406,684, filed on Sep. 14, 2022, the content of which is incorporated herein by reference.

### BACKGROUND OF THE INVENTION

Pathological accumulation of glycogen is a hallmark of several devastating and chronic human diseases. For some of these disorders, the cellular etiology driving this aberrant accumulation has clear genetic underpinnings and for others the mechanistic driving force is more complex. Nonetheless, the consequence of elevated levels of glycogen is altered cellular homeostasis and impaired tissue function over time. The rate limiting enzyme in the glycogen synthesis pathway is the protein Glycogen Synthase (GYS). In humans there are two isoforms GYS1 & GYS2. The former is ubiquitously expressed but highly abundant in muscle cells, while the latter is expressed exclusively in liver. Glycogen synthesis ultimately begins with transport of glucose into cells via the GLUT transporter family of proteins. Conversion of glucose into glycogen follows along a well characterized biochemical conversion pathway to the step where GYS covalently links glucose molecules into long branches via α1,4-glycosidic linkages. The final spherical structure of glycogen results from the action of Glycogen Branching Enzyme (GBE) which introduces α1,6-linkage branch points along the strands. The result of this biochemical chain of events is the generation of an energy dense and highly soluble molecule that can be stored in the cytosol of cells for rapid catabolism into glucose energy when needed.

An imbalance in the equilibrium of either glycogen synthesis or glycogenolysis can result in aberrant accumulation of cellular stores of glycogen. It has long been hypothesized that substrate reduction therapy targeted to inhibit glycogen synthase could be an effective treatment for diseases of glycogen storage. Indeed, substrate reduction therapy drugs have been very successful in modulating patient disease course in other storage disorders including Gaucher and Fabry diseases (Platt F M, Butters T D. Substrate Reduction Therapy. Lysosomal Storage Disorders, Springer US chapter 11, pgs 153-168, 2007; Shemesh E, et al. Enzyme replacement and substrate reduction therapy for Gaucher disease. Cochrane Database of Systematic Reviews, Issue 3, 2015). It is the aim of this invention to inhibit glycogen synthase enzyme activity resulting in reduction of tissue glycogen stores with therapeutic benefit to patients suffering the consequences of aberrant cellular glycogen accumulation.

Pompe Disease is a rare genetic disorder caused by the pathological buildup of cellular glycogen due to loss of function (LOF) mutations in the lysosomal enzyme α-glucosidase (GAA). GAA catabolizes lysosomal glycogen and in its absence, glycogen builds up in lysosomes. This triggers a disease cascade beginning with lysosome and autophagosome dysfunction, leading ultimately to cell death and muscle atrophy over time (Raben N, et al. Autophagy and mitochondria in Pompe Disease: nothing is so new as what has long been forgotten. American Journal oMedical Genetics, vol. 160, 2012. van der Ploeg A T and Reuser A J J, Pompe's Disease. Lancet vol. 372, 2008). In humans, the clinical manifestation of the disease results in a spectrum of severity and occurs at a prevalence of one in 40,000 live births (Meena N K, Raben N. Pompe disease: new developments in an old lysosomal storage disorder. Biomolecules, vol. 10, 2020). Infantile onset patients are born with cellular pathology and rapidly develop severe impairments including myopathy, heart defects, organomegaly, and hypotonia which collectively left untreated will take the child's life within a year. The later onset children may develop heart enlargement but are characterized consistently by the progressive loss of motor function, degeneration of skeletal muscle, and ultimate failure of the respiratory system leading to early death. Late onset adult Pompe patients exhibit normal heart function but develop progressive muscle weakness and respiratory decline then failure. The current standard of care for Pompe patients is enzyme replacement therapy (ERT) with recombinant human GAA. ERT treatment has been successful in slowing the rate of disease progression but in the majority of patients there remains incredible unmet need (Schoser B, et al. The humanistic burden of Pompe disease: are there still unmet needs?A systematic review. BMC Neurology, vol. 17, 2017). For over a decade, substrate reduction therapy targeting GYS1 has been hypothesized to be beneficial for the treatment of Pompe disease. In fact, three separate preclinical modalities have demonstrated that GYS1 genetic LOF in Pompe model mice effectively reduces tissue glycogen and improves mouse disease outcomes (Douillard-Guilloux G, et al. Modulation of glycogen synthesis by RNA interference: towards a new therapeutic approach for glycogenosis type II. Human Molecular Genetics, vol. 17, no. 24, 2008; Douillard-Guilloux G, et al. Restoration of muscle functionality by genetic suppression of glycogen synthesis in a murine model of Pompe disease. Human Molecular Genetics, vol. 19, no. 4, 2010; Clayton N P, et al. Antisense oligonucleotide-mediated suppression of muscle glycogen synthase 1 synthesis as an approach for substrate reduction therapy of Pompe Disease. Molecular Therapy—Nucleic Acids, vol. 3, 2014). A small molecule GYS1 inhibitor could be used to address the current unmet needs for Pompe patients either as a single therapy or in combination with standard of care ERT.

Pompe disease is only one of more than a dozen diseases caused by an inborn error of metabolism that result in aberrant build-up of glycogen in various tissues of the body. For some glycogen storage diseases (GSDs), specific dietary regimes effectively manage the disease but for others there are no clinically approved therapeutic interventions to modify disease course. Therefore, inhibition of glycogen synthesis and the concomitant reduction in tissue glycogen levels may be a viable treatment option for these patients. Cori disease, GSD III, is caused by mutations in the glycogen debranching enzyme (GDE) which results in pathological glycogen accumulation in the heart, skeletal muscle, and liver (Kishnani P, et al. Glycogen storage disease type III diagnosis and management guidelines. Genetics in Medicine, vol. 12, no. 7, 2010). While dietary management can be effective in ameliorating aspects of the disease there is currently no treatment to prevent the progressive myopathy in GSD III. Adult polyglucosan body disease (APBD) is an adult-onset disorder caused by loss of activity in the glycogen branching enzyme (GBE1). Deficiency in GBE results in accumulation of long strands of unbranched glycogen which precipitate in the cytosol generating polyglucosan bodies, and ultimately triggering neurological deficits in both the central and peripheral nervous systems. Genetic deletion of GYS1 in the APBD mouse model rescued deleterious accumulation of glycogen, improved life span, and neuromuscular function (Chown E E, et al. GYS1 or PPP1R3C deficiency rescues murine adult polyglucosan body disease. Annals of Clinical and Translational Neurology, vol. 7, no. 11, 2020). Lafora Disease (LD) is a very debilitating juvenile onset epilepsy disorder also characterized by accumulation of polyglucason bodies. Genetic cross of LD mouse models with GYS1 knock out (KO) mice resulted in rescue of disease phenotypes (Pedersen B, et al. Inhibiting glycogen synthesis prevents Lafora disease in a mouse model. Annals of Neurology, vol. 74, no. 2, 2013; Varea O, et al. Suppression of glycogen synthesis as a treatment for Lafora disease: establishing the window of opportunity. Neurobiology of Disease, 2020).

The reliance on high levels of glycogen by clear cell cancers has recently emerged as a novel therapeutic target. Ewing sarcoma (ES), clear cell renal cell carcinoma (ccRCC), glycogen rich clear cell carcinoma breast cancer (GRCC), acute myeloid leukemia (AML), and nonsmall-cell lung carcinoma (NSCLC) are all examples of cancers histopathologically defined by PAS+abnormally high levels of cellular glycogen. Elevated transcriptional levels of GYS1 have been significantly correlated with poor disease outcomes in NSCLC (Giatromanolaki A, et al. Expression of enzymes related to glucose metabolism in non-small cell lung cancer and prognosis. Experimental Lung Research, vol. 43, no. 4-5, 2017) and AML (Falantes J F, et al. Overexpression of GYS1, MIF, and MYC is associated with adverse outcome and poor response to azacitidine in myelodysplastic syndromes and acute myeloid leukemia. Clinical Lymphoma, Myeloma & Leukemia, vol. 15, no. 4, 2015). Lentiviral knockdown of GYS1 in cultured myeloid leukemia cells potently inhibited in vitro cancer cell growth and in vivo tumorigenesis (Bhanot H, et al. Pathological glycogenesis through glycogen synthase I and suppression of excessive AMP kinase activity in myeloid leukemia cells. Leukemia, vol. 29, no. 7, 2015). Genetic knock-down of GYS1 in ccRCC cell models both suppresses tumor growth in vivo and increases the synthetic lethality of sunitinub (Chen S, et al. GYS1 induces glycogen accumulation and promotes tumor progression via the NF-kB pathway in clear cell renal carcinoma. Theranostics, vol. 10, no. 20, 2020).

Reduction of GYS1 enzyme activity and reduced cellular stores of glycogen in preclinical models of Pompe disease, APBD, LD, AML, ccRCC, and NSCLC all provide compelling evidence of the potential therapeutic benefit of inhibiting glycogen synthesis. It is the aim of this invention to inhibit glycogen synthase enzyme activity resulting in reduction of tissue glycogen stores with therapeutic benefit to patients suffering the consequences of accumulated cellular glycogen.

### BRIEF SUMMARY OF THE INVENTION

In one aspect, provided herein is a compound of formula (I):

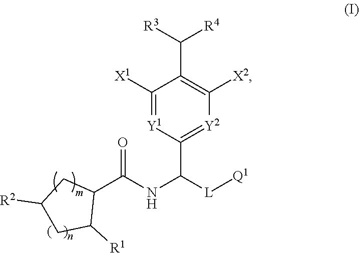

or a stereoisomer or tautomer thereof, or a pharmaceutically acceptable salt of any of the foregoing, wherein:
Y¹ and Y² are each CH, or
one of Y¹ and Y² is N and the other of Y¹ and Y² is CH;
X¹ and X² are each independently H or halo;
R³ and R⁴ are each —CH₃, or
R³ and R⁴ are taken, together with the atoms to which they are attached, to form cyclopropyl or cyclobutyl;
either
(1) L is absent; and
Q¹ is:
(i) C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
   the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
   the 3-15 membered heterocyclyl of the —NH-(3-15 membered heterocyclyl) is optionally substituted with one or more oxo or C₁₋₆alkyl, or
(ii) 3-15 membered heterocyclyl, wherein the 3-15 membered heterocyclyl of Q¹ is optionally substituted with one or more oxo, or
(iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl;
or
(2) L is —CH₂—; and
Q¹ is C₃₋₁₀cycloalkyl;
m is 0 or 1;
n is 0 or 1;
R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein
   the C₁₋₆alkyl of the —C(O)—NH(C₁₋₆alkyl) of R¹ is optionally substituted with one or more —C(O)—C₁₋₆alkoxy, and
   the C₁₋₆alkyl of the —NH—C(O)—C₁₋₆alkyl of R¹ is optionally substituted with one or more —NH—C(O)—C₁₋₆alkyl or —C(O)—NH₂; and
R² is H, halo, or —OH.

In one aspect, provided herein is a compound of formula (I-A):

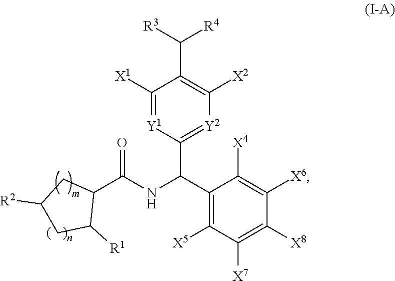

or a stereoisomer or tautomer thereof, or a pharmaceutically acceptable salt of any of the foregoing, wherein m, n, R¹, R², R³, R⁴, X¹, X², X⁴, X⁵, X⁶, X⁷, X⁸, Y¹, Y², and R^{a} are as defined elsewhere herein.

In one aspect, provided herein is a compound of formula (I-B):

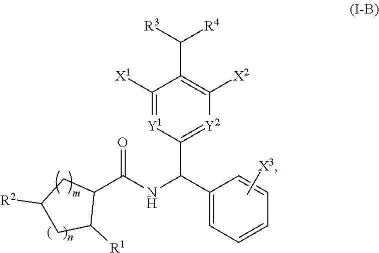

or a stereoisomer or tautomer thereof, or a pharmaceutically acceptable salt of any of the foregoing, wherein m, n, R¹, R², R³, R⁴, X¹, X², X³, Y¹, Y², and R^{a} are as defined elsewhere herein.

In one aspect, provided herein is a compound of formula (I-C):

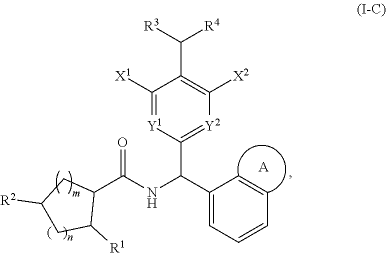

or a stereoisomer or tautomer thereof, or a pharmaceutically acceptable salt of any of the foregoing, wherein m, n, R¹, R², R³, R⁴, X¹, X², Y¹, Y², R^{a} and ring A are as defined elsewhere herein.

In one aspect, provided herein is a compound of formula (I-D):

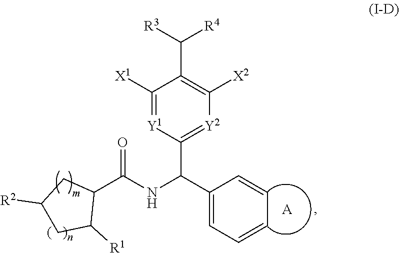

or a stereoisomer or tautomer thereof, or a pharmaceutically acceptable salt of any of the foregoing, wherein m, n, R¹, R², R³, R⁴, X¹, X², Y¹, Y², R^{a} and ring A are as defined elsewhere herein.

In one aspect, provided is a compound of formula (I-E):

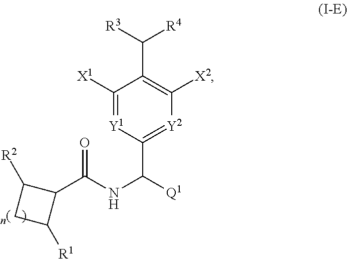

or a stereoisomer or tautomer thereof, or a pharmaceutically acceptable salt of any of the foregoing, wherein n, X¹, X², R¹, R², R³, R Y¹, Y², R^{a} and Q¹ are as defined elsewhere herein.

In one aspect, provided is a compound of formula (I-F):

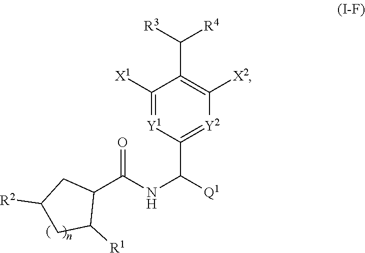

or a stereoisomer or tautomer thereof, or a pharmaceutically acceptable salt of any of the foregoing, wherein n, X¹, X², R¹, R², R³, R⁴, Y¹, Y², R^{a} and Q¹ are as defined elsewhere herein.

In one aspect, provided herein is a compound of formula (I-G):

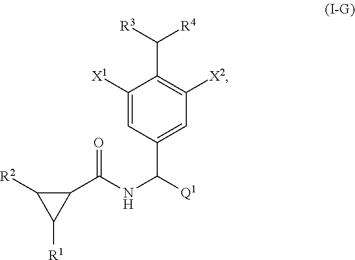

or a stereoisomer or tautomer thereof, or a pharmaceutically acceptable salt of any of the foregoing, wherein X¹, X², R¹, R², R³, R⁴, Y¹, Y², R^{a} and Q¹ are as defined elsewhere herein.

In one aspect, provided herein is a compound of formula (I-H):

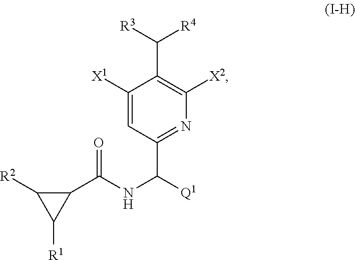

or a stereoisomer or tautomer thereof, or a pharmaceutically acceptable salt of any of the foregoing, wherein X¹, X², R¹, R², R³, R⁴, Y¹, Y², R^{a} and Q¹ are as defined elsewhere herein.

In one aspect, provided herein is a pharmaceutical composition, comprising (i) a compound of formula (I), or any variation or embodiment thereof, or a stereoisomer or tautomer thereof, or a pharmaceutically acceptable salt of any of the foregoing, and (ii) one or more pharmaceutically acceptable excipients.

In one aspect, provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of inhibiting GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of reducing tissue glycogen stores in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of modulating GYS1 in a cell of an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a GYS1-mediated disease, disorder, or condition in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a GYS1-mediated disease, disorder, or condition in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a kit, comprising (i) a composition comprising an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an GYS1-mediated disease, disorder, or condition in an individual in need thereof.

In one aspect, provided herein is a kit, comprising (i) a composition comprising a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an GYS1-mediated disease, disorder, or condition in an individual in need thereof.

In some aspect, provided herein are methods of preparing a compound of formula (I), or any embodiment or variation thereof, such as a compound of formula (I), (I-A), (I-A1), (I-A2), (I-B), (I-B1), (I-B2), (I-B3), (I-B4), (I-B5), (I-B6), (I-C), (I-D), (I-E), (I-E1), (I-E2), (I-F), (I-F1), (I-G), (I-G1), (I-G2), (I-G3), (I-G4), (I-H), (I-H1), (1-H2), (I-H3), (I-H4), or (I-H5), or a stereoisomer or tautomer thereof, or a pharmaceutically acceptable salt of any of the foregoing.

### BRIEF DESCRIPTION OF THE DRAWINGS

FIG. 1 depicts the pathway in which PPP1R3A Loss of Function (LoF) leads to reduction in muscle glycogen.

FIGS. 2A and 2B depict the association between PPP1R3A protein truncating variant (PTV) and left ventricular ejection (LVEF) (%) and left ventricle wall thickness (mm) in UK Biobank.

FIGS. 2C and 2D depict the association between PPP1R3A protein truncating variant (PTV) and exercise output (watts) and max heart rate (HR) exercise (bpm) in UK Biobank.

FIGS. 2E and 2F depict the association between PPP1R3A protein truncating variant (PTV) and PQ interval (ms) and QRS duration (ms) in UK Biobank.

FIGS. 2G and 2H depict the association between PPP1R3A protein truncating variant (PTV) and QT interval (ms) and serum glucose (mmol/L) in UK Biobank.

### DETAILED DESCRIPTION OF THE INVENTION

“Individual” refers to mammals and includes humans and non-human mammals. Examples of individuals include, but are not limited to, mice, rats, hamsters, guinea pigs, pigs, rabbits, cats, dogs, goats, sheep, cows, and humans. In some embodiments, individual refers to a human.

As used herein, “about” a parameter or value includes and describes that parameter or value per se. For example, “about X” includes and describes X per se.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated univalent hydrocarbon chain. As used herein, alkyl has 1-20 carbons (i.e., C₁₋₂₀alkyl), 1-16 carbons (i.e., C₁₋₁₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkenyl”, as used herein, refers to a branched or unbranched univalent hydrocarbon chain comprising at least one carbon-carbon double bond. As used herein, alkenyl has 2-20 carbons (i.e., C₂₋₂₀alkenyl), 2-16 carbons (i.e., C₂₋₁₆alkenyl), 2-12 carbons (i.e., C₂₋₁₂alkenyl), 2-10 carbons (i.e., C₂₋₁₀alkenyl), 2-8 carbons (i.e., C₂₋₈alkenyl), 2-6 carbons (i.e., C₂₋₆alkenyl), 2-4 carbons (i.e., C₂₋₄alkenyl), or 2-3 carbons (i.e., C₂₋₃alkenyl). Examples of alkenyl include, but are not limited to, ethenyl, prop-1-enyl, prop-2-enyl 1,2-butadienyl, and 1,3-butadienyl. When an alkenyl residue having a specific number of carbons is named by chemical name or molecular formula, all positional isomers having that number of carbon atoms may be encompassed—for example, “propenyl” includes prop-1-enyl and prop-2-enyl. Certain commonly used alternative names may be used and will be understood by those of ordinary skill in the art. For instance, a divalent group, such as a divalent “alkenyl” group, may be referred to as an “alkenylene”.

The term “alkynyl”, as used herein, refers to a branched or unbranched univalent hydrocarbon chain comprising at least one carbon-carbon triple bond. As used herein, alkynyl has 2-20 carbons (i.e., C₂₋₂₀alkynyl), 2-16 carbons (i.e., C₂₋₁₆alkynyl), 2-12 carbons (i.e., C₂₋₁₂alkynyl), 2-10 carbons (i.e., C₂₋₁₀alkynyl), 2-8 carbons (i.e., C₂₋₈alkynyl), 2-6 carbons (i.e., C₂₋₆alkynyl), 2-4 carbons (i.e., C₂₋₄alkynyl), or 2-3 carbons (i.e., C₂₋₃alkynyl). Examples of alkynyl include, but are not limited to, ethynyl, prop-1-ynyl, prop-2-ynyl, but-1-ynyl, but-2-ynyl, and but-3-ynyl. When an alkynyl residue having a specific number of carbons is named by chemical name or molecular formula, all positional isomers having that number of carbon atoms may be encompassed for example, “propynyl” includes prop-1-ynyl and prop-2-ynyl. Certain commonly used alternative names may be used and will be understood by those of ordinary skill in the art. For instance, a divalent group, such as a divalent “alkynyl” group, may be referred to as an “alkynylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond.

Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbonyl, decalinyl, 7,7-dimethyl-bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo).

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety).

Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The term “oxo”, as used herein, refers to a ═O moiety.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²p, 353 ¹⁸F, ³⁶Cl, ¹²³I, and ¹²⁵I, respectively. In some embodiments, a compound of formula (A) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

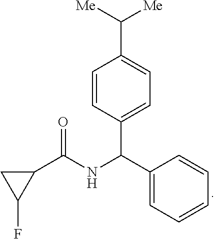 [SMILES: CC(C)c1ccc(C(NC(=O)C2CC2F)c2ccccc2)cc1]

Where enantiomeric and/or diastereomeric forms exist of a given structure, wedged or hashed bonds indicate the composition is made up of at least 90%, by weight, of a single enantiomer or diastereomer with known stereochemistry, e.g.,

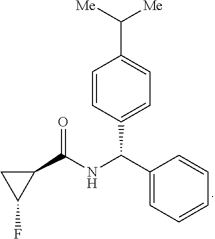 [SMILES: CC(C)c1ccc([C@@H](NC(=O)[C@@H]2C[C@H]2F)c2ccccc2)cc1]

Where relevant, combinations of the above notation may be used. Exemplified species may contain stereogenic centers with known stereochemistry and stereogenic centers with unknown stereochemistry, e.g.,

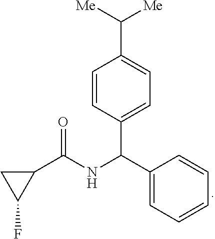 [SMILES: CC(C)c1ccc(C(NC(=O)C2C[C@H]2F)c2ccccc2)cc1]

### Compounds

In one aspect, provided is a compound of formula (I):

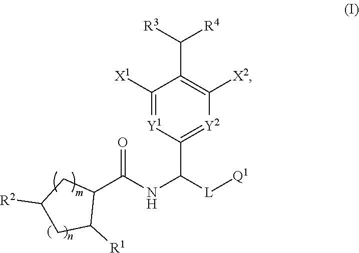

or a stereoisomer or tautomer thereof, or a pharmaceutically acceptable salt of any of the foregoing, wherein:
Y¹ and Y² are each CH, or
one of Y¹ and Y² is N and the other of Y¹ and Y² is CH;
X¹ and X² are each independently H or halo;
R³ and R⁴ are each —CH₃, or
R³ and R⁴ are taken, together with the atoms to which they are attached, to form cyclopropyl or cyclobutyl;
either
(1) L is absent; and
Q¹ is:
(i) C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
   the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
   the 3-15 membered heterocyclyl of the —NH-(3-15 membered heterocyclyl) is optionally substituted with one or more oxo or C₁₋₆alkyl, or
(ii) 3-15 membered heterocyclyl, wherein the 3-15 membered heterocyclyl of Q¹ is optionally substituted with one or more oxo, or
(iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl;
or
(2) L is —CH₂—; and
Q¹ is C₃₋₁₀cycloalkyl;
m is 0 or 1;
n is 0 or 1;
R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein
   the C₁₋₆alkyl of the —C(O)—NH(C₁₋₆alkyl) of R¹ is optionally substituted with one or more —C(O)—C₁₋₆alkoxy, and
   the C₁₋₆alkyl of the —NH—C(O)—C₁₋₆alkyl of R¹ is optionally substituted with one or more —NH—C(O)—C₁₋₆alkyl or —C(O)—NH₂; and
R² is H, halo, or —OH.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2. In some embodiments, m is 0, and n is 1, or 2. In some embodiments, m is 0, and n is 1. In some embodiments, m is 0, and n is 2. In some embodiments, m is 1, and n is 0, or 1. In some embodiments, m is 1, and n is 0. In some embodiments, m is 1, and n is 1. In some embodiments, m+n is 1. In some embodiments, m+n is 2.

In some embodiments of a compound of formula (I), or a stereoisomer or tautomer thereof, or a pharmaceutically acceptable salt of any of the foregoing, Y¹ and Y² are each CH, or one of Y¹ and Y² is N and the other of Y¹ and Y² is CH. In some embodiments, Y¹ and Y² are each CH. In some embodiments, one of Y¹ and Y² is N and the other of Y¹ and Y² is CH.

In some embodiments of a compound of formula (I), or a stereoisomer or tautomer thereof, or a pharmaceutically acceptable salt of any of the foregoing, X¹ is H.

In some embodiments of a compound of formula (I), or a stereoisomer or tautomer thereof, or a pharmaceutically acceptable salt of any of the foregoing, X¹ is halo. In some embodiments, X¹ is fluoro.

In some embodiments of a compound of formula (I), or a stereoisomer or tautomer thereof, or a pharmaceutically acceptable salt of any of the foregoing, X² is H.

In some embodiments of a compound of formula (I), or a stereoisomer or tautomer thereof, or a pharmaceutically acceptable salt of any of the foregoing, X² is halo. In some embodiments, X² is fluoro.

In some embodiments of a compound of formula (I), or a stereoisomer or tautomer thereof, or a pharmaceutically acceptable salt of any of the foregoing, X¹ and X² are each independently H or halo. In some embodiments, X¹ and X² are each independently H or F. In some embodiments, X¹ and X² are each independently H. In some embodiments, X¹ and X² are each independently halo. In some embodiments, X¹ and X² are each independently F. In some embodiments, one of X¹ and X² is H and the other of X¹ and X² is halo. In some embodiments, one of X¹ and X² is H and the other of X¹ and X² is F.

In some embodiments of a compound of formula (I), or a stereoisomer or tautomer thereof, or a pharmaceutically acceptable salt of any of the foregoing, R³ and R⁴ are each —CH₃. In some embodiments, R³ and R⁴ are taken, together with the atoms to which they are attached, to form cyclopropyl. In some embodiments, R³ and R⁴ are taken, together with the atoms to which they are attached, to form cyclobutyl.

In some embodiments of a compound of formula (I), or a stereoisomer or tautomer thereof, or a pharmaceutically acceptable salt of any of the foregoing, R³ and R⁴ are each —CH₃, and X¹ and X² are each hydrogen. In some embodiments, R³ and R⁴ are each —CH₃, and X¹ or X² is F. In some embodiments, R³ and R⁴ are taken, together with the atoms to which they are attached, to form cyclopropyl and X¹ and X² are each hydrogen. In some embodiments, R³ and R⁴ are taken, together with the atoms to which they are attached, to form cyclobutyl and X¹ and X² are each hydrogen. In some embodiments, R³ and R⁴ are taken, together with the atoms to which they are attached, to form cyclopropyl and X¹ or X² is F.

In some embodiments of a compound of formula (I), or a stereoisomer or tautomer thereof, or a pharmaceutically acceptable salt of any of the foregoing, Y¹ and Y² are each CH, R³ and R⁴ are each —CH₃, and X¹ and X² are each hydrogen. In some embodiments, Y¹ and Y² are each CH, R³ and R⁴ are each —CH₃, and X¹ or X² is F. In some embodiments, Y¹ and Y² are each CH, R³ and R⁴ are taken, together with the atoms to which they are attached, to form cyclopropyl and X¹ and X² are each hydrogen. In some embodiments, Y¹ and Y² are each CH, R³ and R⁴ are taken, together with the atoms to which they are attached, to form cyclobutyl and X¹ and X² are each hydrogen. In some embodiments, Y¹ and Y² are each CH, R³ and R⁴ are taken, together with the atoms to which they are attached, to form cyclopropyl and X¹ or X² is F.

In some embodiments of a compound of formula (I), or a stereoisomer or tautomer thereof, or a pharmaceutically acceptable salt of any of the foregoing, one of Y¹ and Y² is N and the other of Y¹ and Y² is CH, R³ and R⁴ are each —CH₃, and X¹ and X² are each hydrogen. In some embodiments, one of Y¹ and Y² is N and the other of Y¹ and Y² is CH, R³ and R⁴ are each —CH₃, and X¹ or X² is F. In some embodiments, one of Y¹ and Y² is N and the other of Y¹ and Y² is CH, R³ and R⁴ are taken, together with the atoms to which they are attached, to form cyclopropyl and X¹ and X² are each hydrogen. In some embodiments, one of Y¹ and Y² is N and the other of Y¹ and Y² is CH, R³ and R⁴ are taken, together with the atoms to which they are attached, to form cyclobutyl and X¹ and X² are each hydrogen. In some embodiments, one of Y¹ and Y² is N and the other of Y¹ and Y² is CH, R³ and R⁴ are taken, together with the atoms to which they are attached, to form cyclopropyl and X¹ or X² is F.

In some embodiments of a compound of formula (I), or a stereoisomer or tautomer thereof, or a pharmaceutically acceptable salt of any of the foregoing, Y¹ and Y² are each CH.

In some embodiments of a compound of formula (I), or a stereoisomer or tautomer thereof, or a pharmaceutically acceptable salt of any of the foregoing, of formula

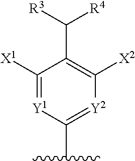

of formula (I) is selected from the group consisting of

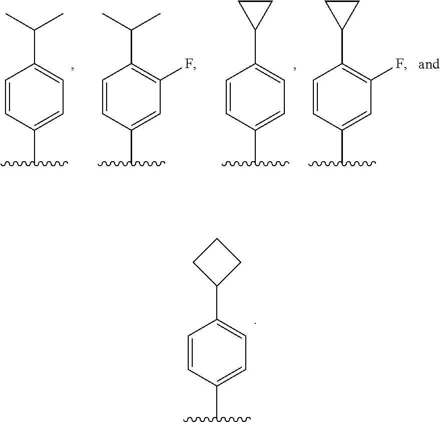

In some embodiments,

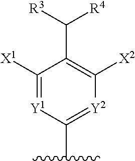

of formula (I) is selected from the group consisting of

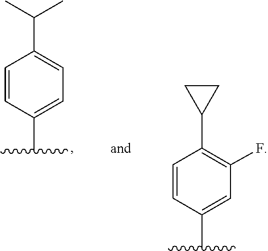

In some embodiments,

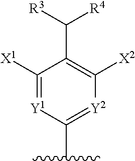

of formula (I) is selected from the group consisting of

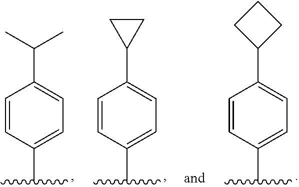

In some embodiments,

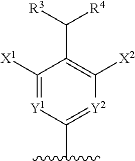

of formula (I) is

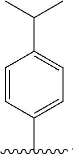 [SMILES: *c1ccc(C(C)C)cc1]

In some embodiments,

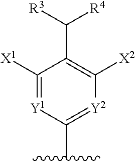

of formula (I) is

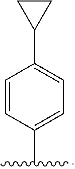 [SMILES: *c1ccc(C2CC2)cc1]

In some embodiments,

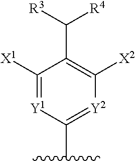

of formula (I) is

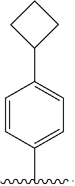 [SMILES: *c1ccc(C2CCC2)cc1]

In some embodiments of a compound of formula (I), or a stereoisomer or tautomer thereof, or a pharmaceutically acceptable salt of any of the foregoing, one of Y¹ and Y² is N and the other of Y¹ and Y² is CH.

In some embodiments of a compound of formula (I), or a stereoisomer or tautomer thereof, or a pharmaceutically acceptable salt of any of the foregoing,

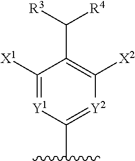

of formula (I) is selected from the group consisting of

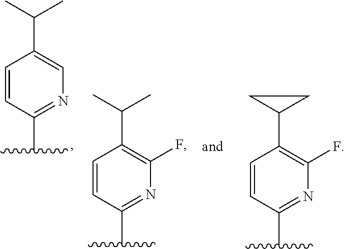

In some embodiments

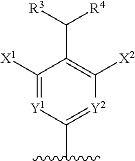

of formula (I) is selected from the group consisting of

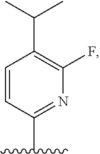 [SMILES: *c1ccc(C(C)C)c(F)n1]

and

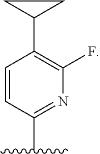 [SMILES: *c1ccc(C2CC2)c(F)n1]

In some embodiments,

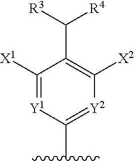

of formula (I) is

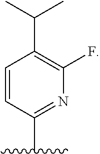 [SMILES: *c1ccc(C(C)C)c(F)n1]

In some embodiments,

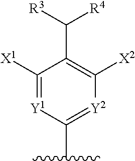

of formula (I) is

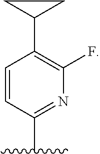 [SMILES: *c1ccc(C2CC2)c(F)n1]

In some embodiments,

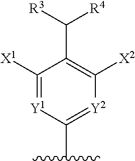

of formula (I) is selected from the group consisting of

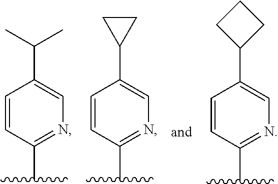

In some embodiments of a compound of formula (I), or a stereoisomer or tautomer thereof, or a pharmaceutically acceptable salt of any of the foregoing, L is absent.

In some embodiments of a compound of formula (I), or a stereoisomer or tautomer thereof, or a pharmaceutically acceptable salt of any of the foregoing, L is absent and Q¹ is C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl.

In some embodiments of a compound of formula (I), or a stereoisomer or tautomer thereof, or a pharmaceutically acceptable salt of any of the foregoing, L is absent and Q¹ is phenyl, wherein the phenyl of Q¹ is optionally substituted with one or more OH, NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), or —NH—C(═N—CN)—NH₂, —NH(C₁₋₆alkyl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

In some embodiments of a compound of formula (I), or a stereoisomer or tautomer thereof, or a pharmaceutically acceptable salt of any of the foregoing, L is absent and Q¹ is phenyl, wherein the phenyl of Q¹ is optionally substituted with one or more OH, NH₂, halo, C₁₋₃alkyl, C₁₋₃alkoxy, C₃₋₆cycloalkyl, 5-10 membered heteroaryl, —NH—C(O)—NH₂, —NH—C(O)—NH(C₁₋₃alkyl), —NH—C(O)—C₁₋₃alkyl, —NH—C(O)—C₃₋₆cycloalkyl, —NH—C(O)-(3-6 membered heterocyclyl), or —NH—C(═N—CN)—NH₂, —NH(C₁₋₃alkyl), wherein the 3-6 membered heterocyclyl of the —NH—C(O)-(3-6 membered heterocyclyl) is optionally substituted with one or more —C(O)—C₁₋₃alkyl or C₁₋₆alkyl, wherein the C₁₋₃alkyl is optionally substituted with one or more halo, C₁₋₃alkoxy, or C₃₋₆cycloalkyl, and the 3-6 membered heterocyclyl of the —NH-(3-6 membered heterocyclyl) is optionally substituted with one or more oxo or C₁₋₃alkyl.

In some embodiments of a compound of formula (I), or a stereoisomer or tautomer thereof, or a pharmaceutically acceptable salt of any of the foregoing, L is absent and Q¹ is selected from the group consisting of

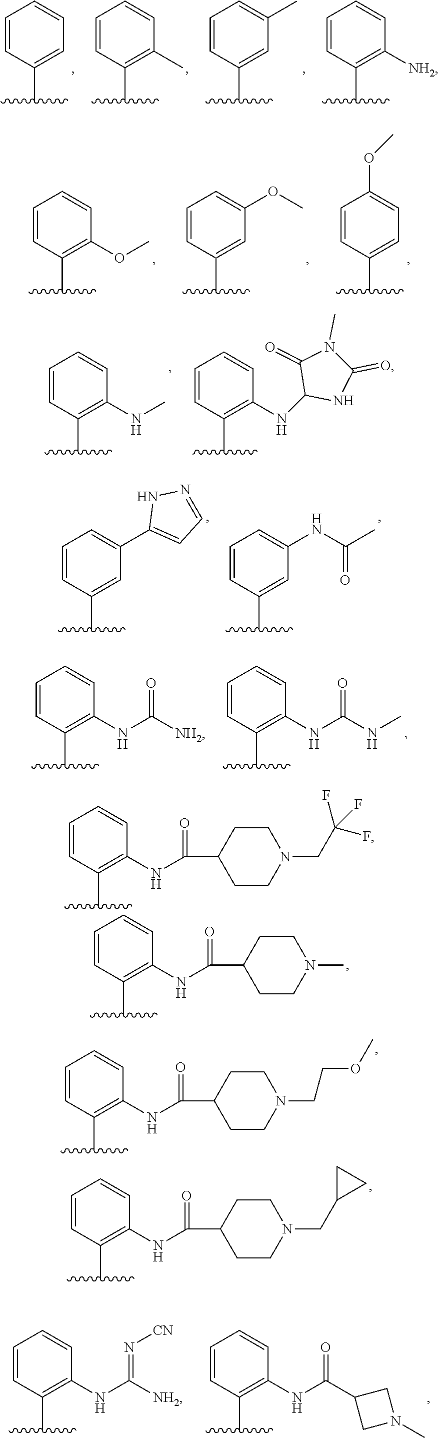

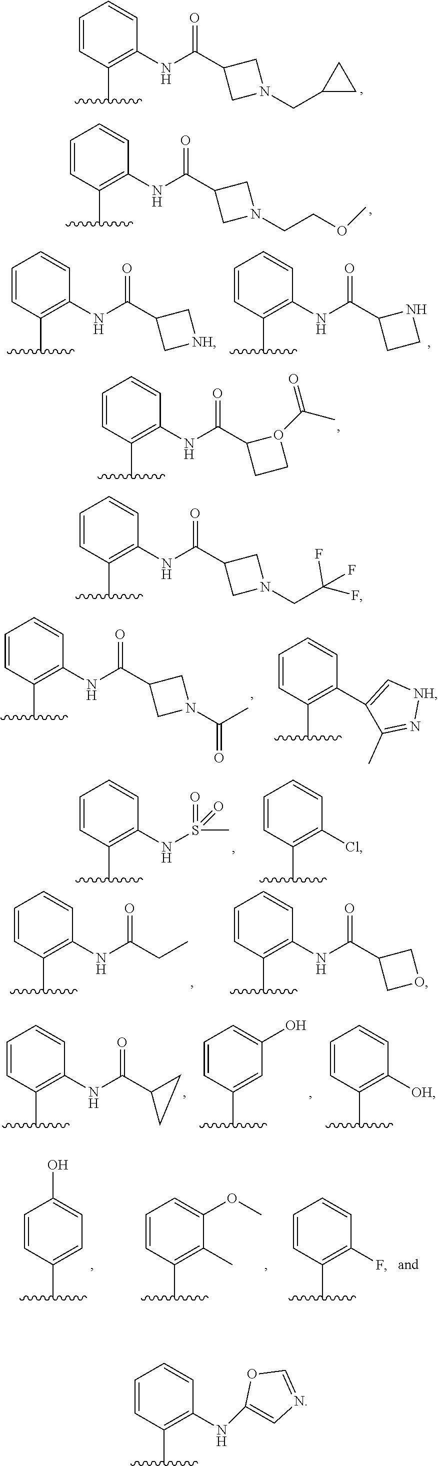

In some embodiments, Q¹ is selected from the group consisting of

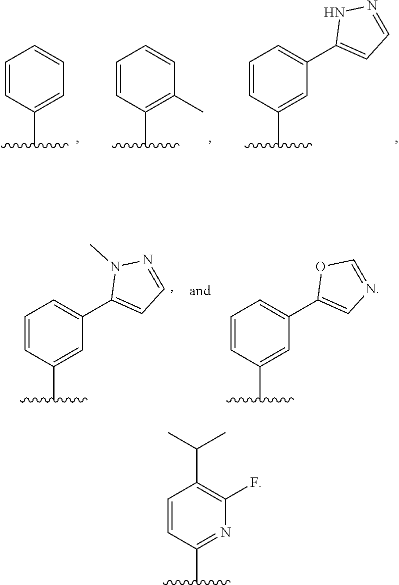

In some embodiments, Q¹ is selected from the group consisting of

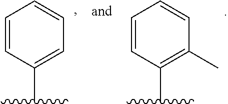

In some embodiments, Q¹ is

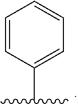 [SMILES: *c1ccccc1]

In some embodiments, Q¹ is

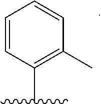 [SMILES: *c1ccccc1C]

In some embodiments of a compound of formula (I), or a stereoisomer or tautomer thereof, or a pharmaceutically acceptable salt of any of the foregoing, L is absent and Q¹ is 3-15 membered heterocyclyl, wherein the 3-15 membered heterocyclyl of Q¹ is optionally substituted with one or more oxo. In some embodiments, L is absent and Q¹ is 3-6 membered heterocyclyl, wherein the 3-6 membered heterocyclyl of Q¹ is optionally substituted with one or more oxo. In some embodiments, Q¹ is 6-10 membered heterocyclyl, wherein the 6-10 membered heterocyclyl of Q¹ is optionally substituted with one or more oxo or C₁₋₃alkyl. In some embodiments, Q¹ is 9-10 membered heterocyclyl, wherein the 9-10 membered heterocyclyl of Q¹ is optionally substituted with one or more oxo. In some embodiments, Q¹ is selected from the group consisting of

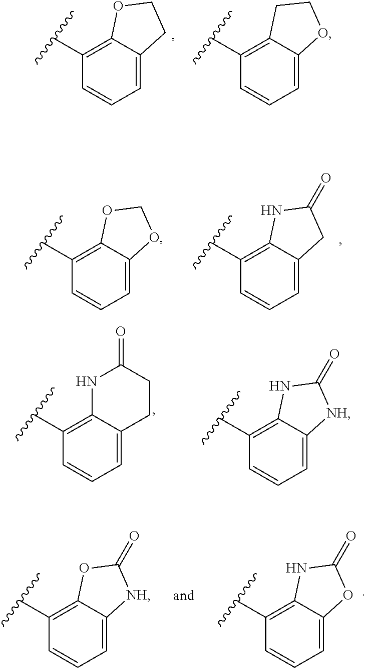

In some embodiments Q¹ is selected from the group consisting of

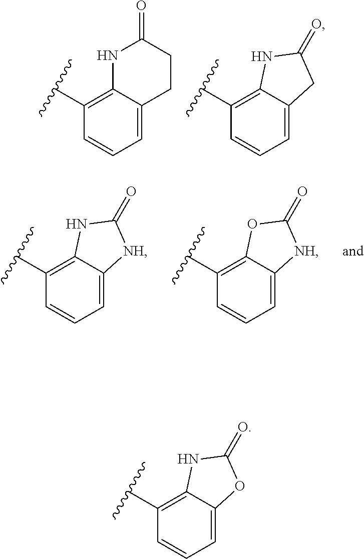

In some embodiments of a compound of formula (I), or a stereoisomer or tautomer thereof, or a pharmaceutically acceptable salt of any of the foregoing, L is absent and Q¹ is 5-20 membered heteroaryl, wherein the 5-20 membered heteroaryl of Q¹ is optionally substituted with one or more —NH₂, halo, C₁₋₆alkyl, or C₃₋₁₀cycloalkyl. In some embodiments, Q¹ is 5-10 membered heteroaryl, wherein the 5-10 membered heteroaryl of Q¹ is optionally substituted with one or more —NH₂, halo, C₁₋₆alkyl, or C₃₋₁₀cycloalkyl, and wherein the 5-10 membered heteroaryl of Q¹ contains at least 1 annular N. In some embodiments, Q¹ is pyridinyl, wherein the pyridinyl of Q¹ is optionally substituted with one or more —NH₂, halo, C₁₋₆alkyl, or C₃₋₁₀cycloalkyl. In some embodiments, Q¹ is pyridinyl. In some embodiments, Q¹ is pyrazolyl.

In some embodiments of a compound of formula (I), or a stereoisomer or tautomer thereof, or a pharmaceutically acceptable salt of any of the foregoing, L is absent and Q¹ is 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl. In some embodiments, Q¹ is 6-10 membered heteroaryl, wherein the 6-10 membered heteroaryl of Q¹ comprises at least one annular N atom and is optionally substituted with one or more —NH₂, halo, C₁₋₆alkyl, or C₃₋₁₀cycloalkyl. In some embodiments, Q¹ is selected from the group consisting of

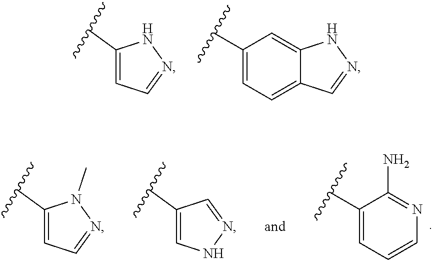

In some embodiments, Q¹ is selected from the group consisting of

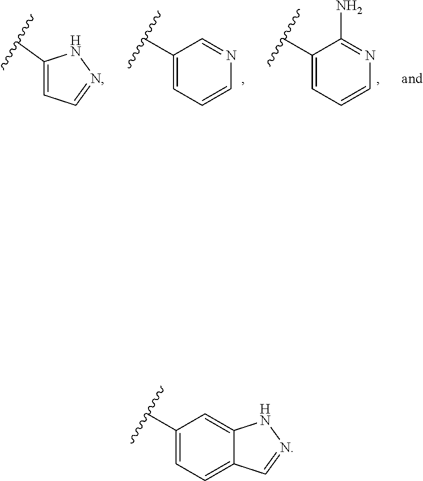 [SMILES: *c1ccn[nH]1 ; *c1cccnc1 ; *c1cccnc1N ; *c1ccc2cn[nH]c2c1]

In some embodiments, Q¹ is selected from the group consisting of

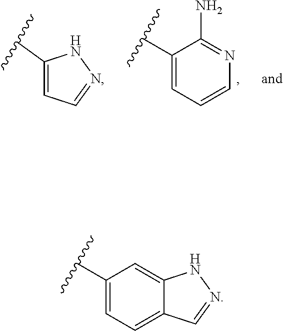 [SMILES: *c1ccn[nH]1 ; *c1cccnc1N ; *c1ccc2cn[nH]c2c1]

In some embodiments of a compound of formula (I), or a stereoisomer or tautomer thereof, or a pharmaceutically acceptable salt of any of the foregoing, L is —CH₂—.

In some embodiments of a compound of formula (I), or a stereoisomer or tautomer thereof, or a pharmaceutically acceptable salt of any of the foregoing, L is —CH₂— and Q¹ is C₃₋₁₀cycloalkyl. In some embodiments, L is —CH₂— and Q¹ is C₃₋₆cycloalkyl.

In some embodiments of a compound of formula (I), or a stereoisomer or tautomer thereof, or a pharmaceutically acceptable salt of any of the foregoing, m is 0, or 1, and n is 0, or 1. In some embodiments, m is 0, and n is 1. In some embodiments, m is 0, and n is 0. In some embodiments, m is 0, n is 1 or 0. In some embodiments, m is 1, and n is 0. In some embodiments, m is 1, and n is 1. In some embodiments, m is 1, and n is 0 or 1.

In some embodiments of a compound of formula (I), or a stereoisomer or tautomer thereof, or a pharmaceutically acceptable salt of any of the foregoing, R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂ and R² is H, halo, or —OH. In some embodiments, R¹ is H, halo, —CN, —C(O)—NH₂, —C(O)—NH(CN), —C(O)—NH(C₁₋₃alkyl), —NH—C(O)—NH₂, or —NH—C(O)—C₁₋₆alkyl, wherein the C₁₋₃alkyl of the —C(O)—NH(C₁₋₃alkyl) of R¹ is optionally substituted with one or more —C(O)—C₁₋₃alkoxy, and the C₁₋₃alkyl of the —NH—C(O)—C₁₋₃alkyl of R¹ is optionally substituted with one or more —NH—C(O)—C₁₋₃alkyl or —C(O)—NH₂. In some embodiments,

In some embodiments of a compound of formula (I), or a stereoisomer or tautomer thereof, or a pharmaceutically acceptable salt of any of the foregoing, R¹ is selected from the group consisting of H, —CN, —C(O)—NH₂, —C(O)—NH(CN), —C(O)—NH(C₁₋₆alkyl), —NH—C(O)—NH₂, and —NH—C(O)—C₁₋₆alkyl, wherein
the C₁₋₆alkyl of the —C(O)—NH(C₁₋₆alkyl) is optionally substituted with one or more —C(O)—C₁₋₆alkoxy, and
the C₁₋₆alkyl of the —NH—C(O)—C₁₋₆alkyl is optionally substituted with one or more —NH—C(O)—C₁₋₆alkyl or —C(O)—NH₂.

In some embodiments of a compound of formula (I), or a stereoisomer or tautomer thereof, or a pharmaceutically acceptable salt of any of the foregoing, R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂.

In some embodiments of a compound of formula (I), or a stereoisomer or tautomer thereof, or a pharmaceutically acceptable salt of any of the foregoing, R¹ is H.

In some embodiments of a compound of formula (I), or a stereoisomer or tautomer thereof, or a pharmaceutically acceptable salt of any of the foregoing, R¹ is selected from the group consisting of —CN, —C(O)—NH₂, —C(O)—NH(CN), —C(O)—NH(C₁₋₆alkyl), —NH—C(O)—NH₂, and —NH—C(O)—C₁₋₆alkyl, wherein
the C₁₋₆alkyl of the —C(O)—NH(C₁₋₆alkyl) is optionally substituted with one or more —C(O)—C₁₋₆alkoxy, and
the C₁₋₆alkyl of the —NH—C(O)—C₁₋₆alkyl is optionally substituted with one or more —NH—C(O)—C₁₋₆alkyl or —C(O)—NH₂.

In some embodiments of a compound of formula (I), or a stereoisomer or tautomer thereof, or a pharmaceutically acceptable salt of any of the foregoing, wherein R¹ is selected from the group consisting of —CN,

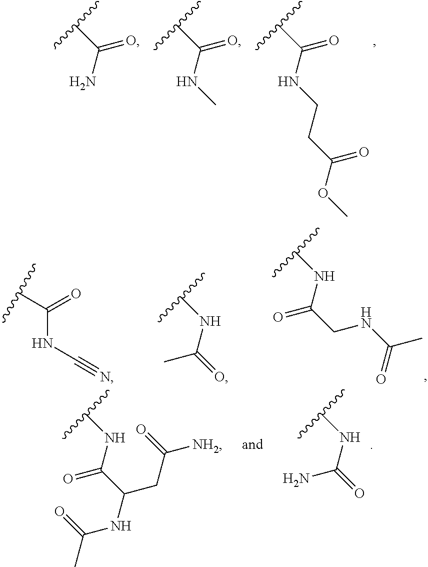

In some embodiments of a compound of formula (I), or a stereoisomer or tautomer thereof, or a pharmaceutically acceptable salt of any of the foregoing, R¹ is H or halo. In some embodiments, R¹ is H or fluoro. In some embodiments, R¹ is H. In some embodiments, R¹ is fluoro.

In some embodiments of a compound of formula (I), or a stereoisomer or tautomer thereof, or a pharmaceutically acceptable salt of any of the foregoing, R² is H, halo, or —OH. In some embodiments, R² is H. In some embodiments, R² is halo. In some embodiments, R² is F. In some embodiments, R² is —OH.

In some embodiments of a compound of formula (I), or any embodiment or variation thereof, such as a compound of formula (I-A), (I-A1), (I-A2), (I-B), (I-B1), (I-B2), (I-B3), (I-B4), (I-B5), (I-B6), (I-C), (I-D), (I-E), (I-E1), (I-E2), (I-F), (I-F1), (I-F2), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing, has a stereochemical configuration of the formula

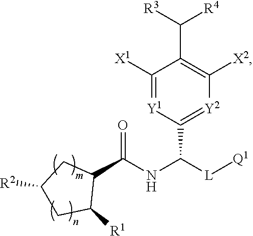

wherein m, n, R¹, R², R³, R⁴, X¹, X², Y¹, Y², L and Q¹, are as defined elsewhere herein.

In some embodiments of a compound of formula (I), or any embodiment or variation thereof, such as a compound of formula (I-A), (I-A1), (I-A2), (I-B), (I-B1), (I-B2), (I-B3), (I-B4), (I-B5), (I-B6), (I-C), (I-D), (I-E), (I-E1), (I-E2), (I-F), (I-F1), (I-F2), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing, has a stereochemical configuration of the formula

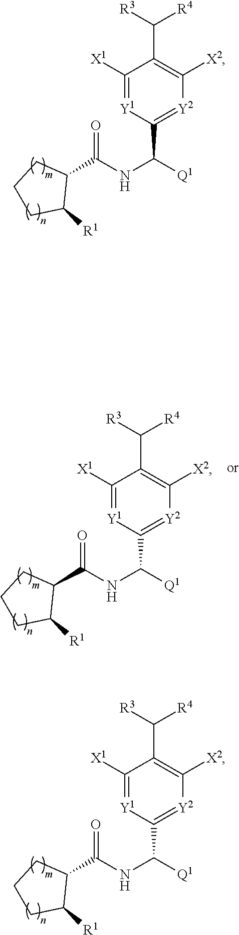

wherein m, n, R¹, R², R³, R⁴, X¹, X², Y¹, Y², and Q¹ are as defined elsewhere herein.

In some embodiments of a compound of formula (I), or a stereoisomer or tautomer thereof, or a pharmaceutically acceptable salt of any of the foregoing, m is 0, or 1; n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is phenyl wherein the phenyl of Q¹ is optionally substituted with one or more OH, NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl; R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂; and R² is H, halo, or —OH. In some embodiments, m is 0, or 1, and n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is phenyl wherein the phenyl of Q¹ is optionally substituted with one or more OH, NH₂, halo, C₁₋₃alkyl, C₁₋₃alkoxy, C₆₋₁₀cycloalkyl, 5-10 membered heteroaryl, —NH—C(O)—NH₂, —NH—C(O)—NH(C₁₋₃alkyl), —NH—C(O)—C₁₋₃alkyl, —NH—C(O)—C₃₋₆cycloalkyl, —NH—C(O)-(3-6 membered heterocyclyl), —NH—C(═N—CN)—NH₂, —NH(C₁₋₃alkyl), —NH-(3-10 membered heterocyclyl), or —NH-(5-10 membered heteroaryl), wherein the 3-10 membered heterocyclyl of the —NH—C(O)-(3-10 membered heterocyclyl) is optionally substituted with one or more —C(O)—C₁₋₃alkyl or C₁₋₃alkyl, wherein the C₁₋₃alkyl is optionally substituted with one or more halo, C₁₋₃alkoxy, or C₆₋₁₀cycloalkyl, and the 3-10 membered heterocyclyl of the —NH-(3-10 membered heterocyclyl) is optionally substituted with one or more oxo or C₁₋₃alkyl; R¹ is H, halo, —CN, —C(O)—NH₂, —C(O)—NH(CN), —C(O)—NH(C₁₋₃alkyl), —NH—C(O)—NH₂, or —NH—C(O)—C₁₋₃alkyl, wherein the C₁₋₃alkyl of the —C(O)—NH(C₁₋₃alkyl) of R¹ is optionally substituted with one or more —C(O)—C₁₋₃alkoxy, and the C₁₋₃alkyl of the —NH—C(O)—C₁₋₃alkyl of R¹ is optionally substituted with one or more —NH—C(O)—C₁₋₃alkyl or —C(O)—NH₂; and R² is H, halo, or —OH.

In some embodiments, m is 0, or 1, and n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is phenyl wherein the phenyl of Q¹ is optionally substituted with one or more OH, NH₂, F, CH₃, —OCH₃, C₆₋₁₀cycloalkyl, 5-10 membered heteroaryl, —NH—C(O)—NH₂, —NH—C(O)—NH(CH₃), —NH—C(O)— CH₃, —NH—C(O)—C₃₋₆cycloalkyl, —NH—C(O)-(3-6 membered heterocyclyl), —NH—C(═N—CN)—NH₂, —NH(C₁₋₃alkyl), —NH-(3-10 membered heterocyclyl), or —NH-(5-10 membered heteroaryl), wherein the 3-10 membered heterocyclyl of the —NH—C(O)-(3-6 membered heterocyclyl) is optionally substituted with one or more —C(O)—CH₃ or CH₃, wherein the CH₃ is optionally substituted with one or more halo, —OCH₃, or C₆₋₁₀cycloalkyl, and the 3-6 membered heterocyclyl of the —NH-(3-6 membered heterocyclyl) is optionally substituted with one or more oxo or C₁₋₃alkyl; R¹ is H, F, —CN, —C(O)—NH₂, —C(O)—NH(CN), —C(O)—NH(CH₃), —NH—C(O)—NH₂, or —NH—C(O)— CH₃, wherein the CH₃ of the —C(O)—NH(CH₃) of R¹ is optionally substituted with one or more —C(O)—OCH₃, and the —CH₃ of the —NH—C(O)—CH₃ of R¹ is optionally substituted with one or more —NH—C(O)—CH₃ or —C(O)—NH₂; and R² is H, halo, or —OH.

In some embodiments, m is 0; n is 0; Y¹ and Y² are each CH; R³ and R⁴ are each —CH₃; X¹ is H; X² is H; Q¹ is phenyl wherein the phenyl of Q¹ is optionally substituted with one or more —OH, NH₂, F, CH₃, —OCH₃, C₆₋₁₀cycloalkyl, 5-10 membered heteroaryl, —NH—C(O)—NH₂, —NH—C(O)—NH(CH₃), —NH—C(O)—CH₃, —NH—C(O)—C₃₋₆cycloalkyl, —NH—C(O)-(3-6 membered heterocyclyl), —NH—C(═N—CN)—NH₂, —NH(C₁₋₃alkyl), —NH-(3-10 membered heterocyclyl), or —NH-(5-10 membered heteroaryl), wherein the 3-10 membered heterocyclyl of the —NH—C(O)-(3-6 membered heterocyclyl) is optionally substituted with one or more —C(O)—CH₃ or CH₃, wherein the CH₃ is optionally substituted with one or more halo, —OCH₃, or C₆₋₁₀cycloalkyl, and the 3-6 membered heterocyclyl of the —NH-(3-6 membered heterocyclyl) is optionally substituted with one or more oxo or C₁₋₃alkyl; R¹ is H; and R² is H.

In some embodiments, m is 0; n is 0; Y¹ and Y² are each CH; R³ and R⁴ are each —CH₃; X¹ is H; X² is F; Q¹ is phenyl wherein the phenyl of Q¹ is optionally substituted with one or more —NH—C(O)—NH₂; R¹ is H or F; and R² is H.

In some embodiments of a compound of formula (I), or a stereoisomer or tautomer thereof, or a pharmaceutically acceptable salt of any of the foregoing, m is 0; and n is 1; Y¹ is CH; Y² is N; R³ and R⁴ are each —CH₃; X¹ and X² are each independently H; Q¹ is phenyl wherein the phenyl of Q¹ is optionally substituted with one or more C₁₋₃alkyl or —NH—C(O)—C₁₋₃alkyl; R¹ is H; and R² is H. In some embodiments, m is 0; and n is 1; Y¹ is CH; Y² is N; R³ and R⁴ are each —CH₃; X¹ and X² are each independently H; Q¹ is phenyl wherein the phenyl of Q¹ is optionally substituted with one or more —CH₃ or —NH—C(O)—CH₃; R¹ is H; and R² is H.

In some embodiments of a compound of formula (I), or a stereoisomer or tautomer thereof, or a pharmaceutically acceptable salt of any of the foregoing, m is 0; and n is 1; Y¹ is CH; Y² is N; X¹ is H; X² is halo; R³ and R⁴ are taken, together with the atoms to which they are attached, to form cyclopropyl; Q¹ is phenyl wherein the phenyl of Q¹ is optionally substituted with one or more 5-10 membered heteroaryl; R¹ is H; and R² is halo. In some embodiments, m is 0; and n is 1; Y¹ is CH; Y² is N; X¹ is H; X² is halo; R³ and R⁴ are each —CH₃ or R³ and R⁴ are taken, together with the atoms to which they are attached, to form cyclopropyl; Q¹ is phenyl wherein the phenyl of Q¹ is optionally substituted with one or more pyrazolyl; R¹ is H; and R² is F.

In some embodiments of a compound of formula (I), or a stereoisomer or tautomer thereof, or a pharmaceutically acceptable salt of any of the foregoing, m is 0, or 1, and n is 0, or 1; Y¹ and Y² are each CH or one of Y¹ and Y² is N and the other of Y¹ and Y² is CH; X¹ and X² are each independently H or halo; R³ and R⁴ are each —CH₃ or R³ and R⁴ are taken, together with the atoms to which they are attached, to form cyclopropyl or cyclobutyl; Q¹ is phenyl; R¹ is H, F, —C(O)—NH(CH₃), —NH—C(O)—NH₂, or —NH—C(O)—CH₃, wherein the —CH₃ of the —NH—C(O)—CH₃ of R¹ is optionally substituted with one or more —NH—C(O)—CH₃ or —C(O)—NH₂; and R² is H, or halo.

In some embodiments of a compound of formula (I), or a stereoisomer or tautomer thereof, or a pharmaceutically acceptable salt of any of the foregoing, m is 0, or 1, and n is 0, or 1; Y¹ and Y² are each CH or one of Y¹ and Y² is N and the other of Y¹ and Y² is CH; X¹ and X² are each independently H or halo; R³ and R⁴ are each —CH₃ or R³ and R⁴ are taken, together with the atoms to which they are attached, to form cyclopropyl or cyclobutyl; Q¹ is phenyl; R¹ is H, F, —C(O)—NH(CH₃), —NH—C(O)—NH₂, or —NH—C(O)—CH₃, wherein the —CH₃ of the —NH—C(O)—CH₃ of R¹ is optionally substituted with one or more —NH—C(O)—CH₃ or —C(O)—NH₂; and R² is H, OH, or halo.

In some embodiments of a compound of formula (I), or a stereoisomer or tautomer thereof, or a pharmaceutically acceptable salt of any of the foregoing, m is 0, or 1, and n is 0, or 1; Y¹ and Y² are each CH; X¹ and X² are each independently H or halo; R³ and R⁴ are each —CH₃ or R³ and R⁴ are taken, together with the atoms to which they are attached, to form cyclopropyl or cyclobutyl; Q¹ is phenyl; R¹ is H, F, —C(O)—NH(CH₃), —NH—C(O)—NH₂, or —NH—C(O)—CH₃, wherein the —CH₃ of the —NH—C(O)—CH₃ of R¹ is optionally substituted with one or more —NH—C(O)—CH₃ or —C(O)—NH₂; and R² is H, OH, or halo.

In some embodiments of a compound of formula (I), or a stereoisomer or tautomer thereof, or a pharmaceutically acceptable salt of any of the foregoing, m is 0, or 1, and n is 0, or 1; one of Y¹ and Y² is N and the other of Y¹ and Y² is CH; X¹ and X² are each independently H or halo; R³ and R⁴ are taken, together with the atoms to which they are attached, to form cyclopropyl or cyclobutyl; Q¹ is phenyl; R¹ is H, or F; and R² is H, or halo.

In some embodiments of a compound of formula (I), or a stereoisomer or tautomer thereof, or a pharmaceutically acceptable salt of any of the foregoing, m is 0, or 1; n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is (i) 3-15 membered heterocyclyl, wherein the 3-15 membered heterocyclyl of Q¹ is optionally substituted with one or more oxo, or (ii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl; R¹ is H, halo, —CN, —C(O)—NH₂, —C(O)—NH(CN), —C(O)—NH(C₁₋₃alkyl), —NH—C(O)—NH₂, or —NH—C(O)—C₁₋₃alkyl, wherein the C₁₋₃alkyl of the —C(O)—NH(C₁₋₃alkyl) of R¹ is optionally substituted with one or more —C(O)—C₁₋₃alkoxy, and the C₁₋₃alkyl of the —NH—C(O)—C₁₋₃alkyl of R¹ is optionally substituted with one or more —NH—C(O)—C₁₋₃alkyl or —C(O)—NH₂; and R² is H, halo, or —OH. In some embodiments, m is 0, or 1; n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is (i) 3-10 membered heterocyclyl, wherein the 3-10 membered heterocyclyl of Q¹ is optionally substituted with one or more oxo, or (ii) 5-10 membered heteroaryl, wherein the 5-10 membered heteroaryl of Q¹ comprises at least one annular N atom and is optionally substituted with one or more —NH₂, halo, C₁₋₃alkyl, or C₃₋₆cycloalkyl; R¹ is H, halo, —CN, —C(O)—NH₂, —C(O)—NH(CN), —C(O)—NH(C₁₋₃alkyl), —NH—C(O)—NH₂, or —NH—C(O)—C₁₋₃alkyl, wherein the C₁₋₃alkyl of the —C(O)—NH(C₁₋₃alkyl) of R¹ is optionally substituted with one or more —C(O)—C₁₋₃alkoxy, and the C₁₋₃alkyl of the —NH—C(O)—C₁₋₃alkyl of R¹ is optionally substituted with one or more —NH—C(O)—C₁₋₃alkyl or —C(O)—NH₂; and R² is H, halo, or —OH.

In some embodiments of a compound of formula (I), or a stereoisomer or tautomer thereof, or a pharmaceutically acceptable salt of any of the foregoing, m is 0; n is 0; Y¹ and Y² are each CH; X¹ and X² are each independently H; R³ and R⁴ are each —CH₃; Q¹ is 3-15 membered heterocyclyl, wherein the 3-15 membered heterocyclyl of Q¹ is optionally substituted with one or more oxo; R¹ is H, halo; and R² is H, halo, or —OH. In some embodiments, m is 0; n is 0; Y¹ and Y² are each CH; X¹ and X² are each independently H; R³ and R⁴ are each —CH₃; Q¹ is 3-10 membered heterocyclyl, wherein the 3-10 membered heterocyclyl of Q¹ is optionally substituted with one or more oxo; R¹ is H, halo; and R² is H, halo, or —OH.

In some embodiments of a compound of formula (I), or a stereoisomer or tautomer thereof, or a pharmaceutically acceptable salt of any of the foregoing, m is 0; n is 0; Y¹ is CH; Y² is N; X¹ is H; X¹ is H; X² is halo; R³ and R⁴ are each —CH₃; Q¹ is 3-15 membered heterocyclyl, wherein the 3-15 membered heterocyclyl of Q¹ is optionally substituted with one or more oxo; R¹ is H, halo; and R² is H, halo, or —OH. In some embodiments, m is 0; n is 0; Y¹ and Y² are each CH; X¹ and X² are each independently H; R³ and R⁴ are each —CH₃; Q¹ is 3-10 membered heterocyclyl, wherein the 3-10 membered heterocyclyl of Q¹ is optionally substituted with one or more oxo; R¹ is H, halo; and R² is H, halo, or —OH.

In some embodiments of a compound of formula (I), or a stereoisomer or tautomer thereof, or a pharmaceutically acceptable salt of any of the foregoing, m is 0; n is 0; Y¹ is CH; Y² is N; X¹ is H; X¹ and X² are each independently H; R³ and R⁴ are each —CH₃; Q¹ is 3-15 membered heterocyclyl, wherein the 3-15 membered heterocyclyl of Q¹ is optionally substituted with one or more oxo; R¹ is H, halo; and R² is H, halo, or —OH. In some embodiments, m is 0; n is 0; Y¹ and Y² are each CH; X¹ and X² are each independently H; R³ and R⁴ are each —CH₃; Q¹ is 3-10 membered heterocyclyl, wherein the 3-10 membered heterocyclyl of Q¹ is optionally substituted with one or more oxo; R¹ is H, halo; and R² is H, halo, or —OH.

In some embodiments of a compound of formula (I), or a stereoisomer or tautomer thereof, or a pharmaceutically acceptable salt of any of the foregoing, m is 0; n is 0; Y¹ and Y² are each CH; X¹ and X² are each independently H; R³ and R⁴ are each —CH₃; Q¹ is 5-20 membered heteroaryl, wherein the 5-20 membered heteroaryl of Q¹ comprises at least one annular N atom and is optionally substituted with one or more C₁₋₆alkyl; R¹ is H; and R² is H. In some embodiments, m is 0; n is 0; Y¹ and Y² are each CH; X¹ and X² are each independently H; R³ and R⁴ are each —CH₃; Q¹ is 5-10 membered heteroaryl, wherein the 5-10 membered heteroaryl of Q¹ comprises at least one annular N atom and is optionally substituted with one or more C₁₋₃alkyl; R¹ is H; and R² is H.

In some embodiments of a compound of formula (I), or a stereoisomer or tautomer thereof, or a pharmaceutically acceptable salt of any of the foregoing, m is 0; n is 0; Y¹ and Y² are each CH; X¹ is H; X² is halo; R³ and R⁴ are each —CH₃; Q¹ is 5-20 membered heteroaryl, wherein the 5-20 membered heteroaryl of Q¹ comprises at least one annular N atom and is optionally substituted with one or more C₁₋₆alkyl; R¹ is H, or halo; and R² is H, or halo. In some embodiments, m is 0; n is 0; Y¹ and Y² are each CH; X¹ and X² are each independently H; R³ and R⁴ are each —CH₃; Q¹ is 5-10 membered heteroaryl, wherein the 5-10 membered heteroaryl of Q¹ comprises at least one annular N atom and is optionally substituted with one or more —C₁₋₃alkyl; R¹ is H, halo; and R² is H, halo, or —OH.

In some embodiments of a compound of formula (I), or a stereoisomer or tautomer thereof, or a pharmaceutically acceptable salt of any of the foregoing, m is 0; n is 0; Y¹ is CH; Y² is N; X¹ is H; X² is halo; R³ and R⁴ are each —CH₃; Q¹ is 5-20 membered heteroaryl, wherein the 5-20 membered heteroaryl of Q¹ comprises at least one annular N atom and is optionally substituted with one or more —C₁₋₆alkyl; R¹ is H, halo; and R² is H, or halo. In some embodiments, m is 0; n is 0; Y¹ and Y² are each CH; X¹ and X² are each independently H; R³ and R⁴ are each —CH₃; Q¹ is 5-10 membered heteroaryl, wherein the 5-10 membered heteroaryl of Q¹ comprises at least one annular N atom and is optionally substituted with one or more —C₁₋₃alkyl; R¹ is H, or halo; and R² is H, or halo.

In some embodiments of a compound of formula (I), or a stereoisomer or tautomer thereof, or a pharmaceutically acceptable salt of any of the foregoing, m is 0; n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is (i)C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl, or (ii) 3-15 membered heterocyclyl, wherein the 3-15 membered heterocyclyl of Q¹ is optionally substituted with one or more oxo, or (iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl; R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂; and R² is H, halo, or —OH.

In some embodiments of a compound of formula (I), or a stereoisomer or tautomer thereof, or a pharmaceutically acceptable salt of any of the foregoing, m is 1; n is 0, or 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is (i)C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl, or (ii) 3-15 membered heterocyclyl, wherein the 3-15 membered heterocyclyl of Q¹ is optionally substituted with one or more oxo, or (iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl; R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂; and R² is H, halo, or —OH. In some embodiments, m is 1; n 1; Y¹ and Y² are each CH, or one of Y¹ and Y² is N and the other of Y¹ and Y² is CH; X¹ and X² are each independently H or halo; R³ and R⁴ are each —CH₃, or R³ and R⁴ are taken, together with the atoms to which they are attached, to form cyclopropyl or cyclobutyl; Q¹ is (i)C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl; R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein the C₁₋₆alkyl of the —C(O)—NH(C₁₋₆alkyl) of R¹ is optionally substituted with one or more —C(O)—C₁₋₆alkoxy, and the C₁₋₆alkyl of the —NH—C(O)—C₁₋₆alkyl of R¹ is optionally substituted with one or more —NH—C(O)—C₁₋₆alkyl or —C(O)—NH₂; and R² is H, halo, or —OH.

In some embodiments, provided herein is a compound of formula (I), or a stereoisomer or tautomer thereof, or a pharmaceutically acceptable salt thereof, wherein the compound is a compound of formula (I-A):

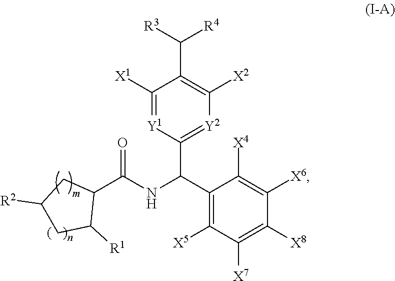

or a stereoisomer or tautomer thereof, or a pharmaceutically acceptable salt of any of the foregoing, wherein, either:
i. X⁴⁻⁸ are each independently H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein
   the 3-9 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
   the 3-9 membered heterocyclyl of the —NH-(3-15 membered heterocyclyl) is optionally substituted with one or more oxo or C₁₋₆alkyl; or
ii. X⁶ is taken together with either of X⁴ or X⁸, and the atoms to which they are attached, to form ring A, wherein ring A is
   3-9 membered heterocyclyl, and wherein the 3-9 membered heterocyclyl of ring A is optionally substituted with one or more oxo, wherein X⁵, X⁷, and the other of X⁴ or X¹ are each independently H, or oxo, or
   5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl, wherein X⁵, X⁷, and the other of X⁴ or X⁸ are each independently H, —NH₂, halo, C₁₋₆alkyl, or C₃₋₁₀cycloalkyl; or
iii. X⁷ is taken together with either of X⁵ or X⁸, and the atoms to which they are attached, to form ring A, wherein ring A is
   3-9 membered heterocyclyl, and wherein the 3-9 membered heterocyclyl of ring A is optionally substituted with one or more oxo, and wherein X⁴, X⁶, and the other of X⁵ or X⁸ are each independently H, or oxo, or
   5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl, and wherein X⁴, X⁶, and the other of X⁵ or X⁸ are each independently H, —NH₂, halo, C₁₋₆alkyl, or C₃₋₁₀cycloalkyl.

In some embodiments of a compound of formula (I-A), or a stereoisomer or tautomer thereof, or a pharmaceutically acceptable salt of any of the foregoing, X⁴⁻⁸ are each independently X⁴⁻⁸ are each independently H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl.

In some embodiments of a compound of formula (I-A), or a stereoisomer or tautomer thereof, or a pharmaceutically acceptable salt of any of the foregoing, X⁴⁻⁸ are each independently H.

In some embodiments of a compound of formula (I-A), or a stereoisomer or tautomer thereof, or a pharmaceutically acceptable salt of any of the foregoing, one of —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.
and the others of X⁴⁻⁸ are each independently H.

In some embodiments, one of X⁴⁻⁸ is selected from the group consisting of methyl, OH, Cl, —OCH₃, NH₂, —NH(CH₃)

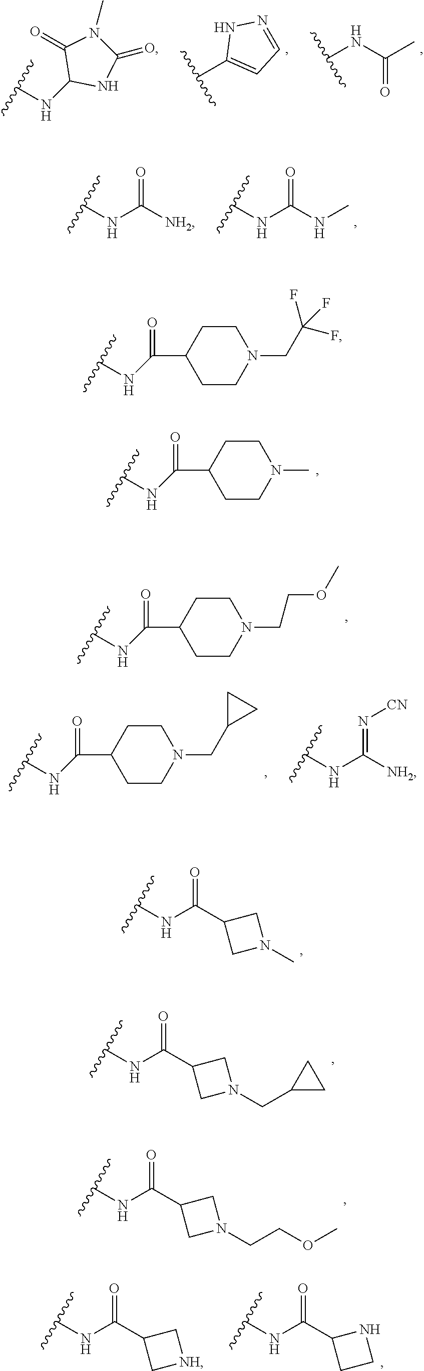

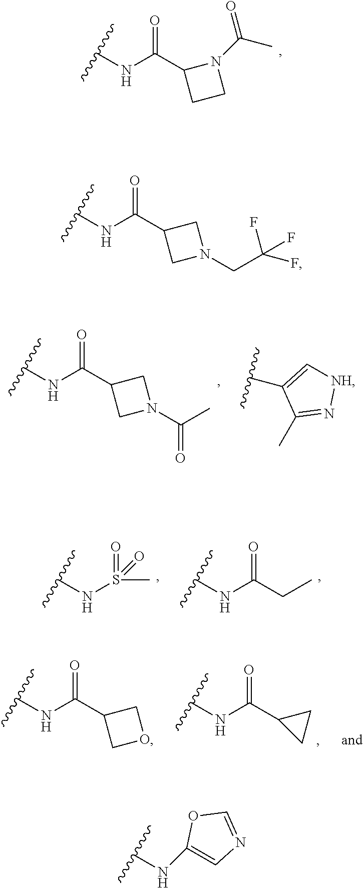

and the others of X⁴⁻⁸ are each independently H.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is
3-9 membered heterocyclyl, wherein the 3-9 membered heterocyclyl of ring A is optionally substituted with one or more oxo, and wherein X⁵, X⁷, and the other of X⁴ or X⁸ are each independently H, or oxo, or
5-14 membered heteroaryl, wherein the 5-14 membered heteroaryl of ring A comprises at least one annular N atom and is optionally substituted with one or more —NH₂, halo, C₁₋₆alkyl, or C₃₋₁₀cycloalkyl, and wherein X⁵, X⁷, and the other of X⁴ or X⁸ are each independently H, —NH₂, halo, C₁₋₆alkyl, or C₃₋₁₀cycloalkyl.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is
3-6 membered heterocyclyl, wherein the 3-9 membered heterocyclyl of ring A is optionally substituted with one or more oxo, and wherein X⁵, X⁷, and the other of X⁴ or X⁸ are each independently H, or oxo, or
5-6 membered heteroaryl, wherein the 5-10 membered heteroaryl of ring A comprises at least one annular N atom and is optionally substituted with one or more —NH₂, halo, C₁₋₆alkyl, or C₃₋₁₀cycloalkyl, and wherein X⁵, X⁷, and the other of X⁴ or X⁸ are each independently H, —NH₂, halo, C₁₋₆alkyl, or C₃₋₁₀cycloalkyl.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is 3-9 membered heterocyclyl, wherein the 3-9 membered heterocyclyl of ring A is optionally substituted with one or more oxo. In some embodiments, ring A is 3-6 membered heterocyclyl, wherein the 3-6 membered heterocyclyl of ring A is optionally substituted with one or more oxo or C₁₋₃alkyl. In some embodiments, ring A is selected from the group consisting of

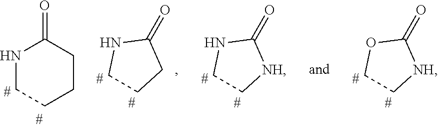

wherein # represents a point of attachment to the rest of the molecule.

In some embodiments of a compound of formula (I-A), or a stereoisomer or tautomer thereof, or a pharmaceutically acceptable salt of any of the foregoing, 5-14 membered heteroaryl, wherein the 5-14 membered heteroaryl of ring A comprises at least one annular N atom and is optionally substituted with one or more —NH₂, halo, C₁₋₆alkyl, or C₃₋₁₀cycloalkyl, and wherein X⁵, X⁷, and the other of X⁴ or X⁸ are each independently H, —NH₂, halo, C₁₋₆alkyl, or C₃₋₁₀cycloalkyl. In some embodiments, ring A is 5-8 membered heteroaryl, wherein the 5-8 membered heteroaryl of ring A is optionally substituted with one or more —NH₂, halo, C₁₋₆alkyl, or C₃₋₁₀cycloalkyl, and wherein X⁵, X⁷, and the other of X⁴ or X⁸ are each independently H, —NH₂, halo, C₁₋₆alkyl, or C₃₋₁₀cycloalkyl. In some embodiments, ring A is

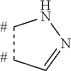

wherein # represents a point of attachment to the rest of the molecule.

In some embodiments, provided herein is a compound of formula (I), or (I-A), or a stereoisomer or tautomer thereof, or a pharmaceutically acceptable salt thereof, wherein the compound is a compound of formula (I-A1):

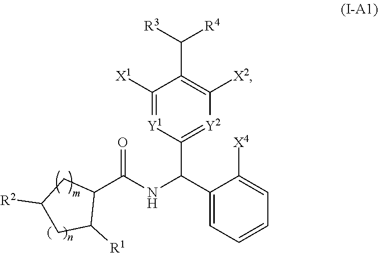

or a stereoisomer or tautomer thereof, or a pharmaceutically acceptable salt of any of the foregoing, wherein X⁴ is H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl.

In some embodiments, provided herein is a compound of formula (I), or (I-A), or a stereoisomer or tautomer thereof, or a pharmaceutically acceptable salt thereof, wherein the compound is a compound of formula (I-A2):

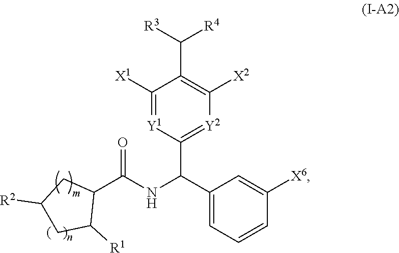

or a stereoisomer or tautomer thereof, or a pharmaceutically acceptable salt of any of the foregoing, wherein X⁶ is H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and the 3-15 membered heterocyclyl of the —NH-(3-15 membered heterocyclyl) is optionally substituted with one or more oxo or C₁₋₆alkyl.

In some embodiments, provided herein is a compound of formula (I), or a stereoisomer or tautomer thereof, or a pharmaceutically acceptable salt thereof, wherein the compound is a compound of formula (I-B):

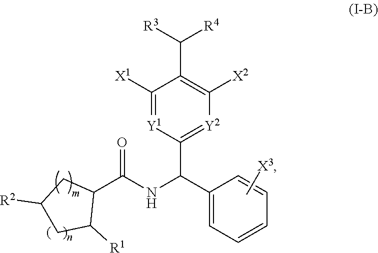

or a stereoisomer or tautomer thereof, or a pharmaceutically acceptable salt of any of the foregoing, wherein X³ is H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

In some embodiments of a compound of formula (I-B), or a stereoisomer or tautomer thereof, or a pharmaceutically acceptable salt of any of the foregoing, X¹ and X² are independently H or halo; and R³ and R⁴ are each —CH₃ or R³ and R⁴ are taken, together with the atoms to which they are attached, to form cyclopropyl or cyclobutyl. In some embodiments, X¹ and X² are independently H or F; and R³ and R⁴ are each —CH₃ or R³ and R⁴ are taken, together with the atoms to which they are attached, to form cyclopropyl or cyclobutyl. In some embodiments, one of X¹ and X² is H; the other of X¹ and X² is halo; and R³ and R⁴ are each —CH₃ or R³ and R⁴ are taken, together with the atoms to which they are attached, to form cyclopropyl or cyclobutyl. In some embodiments, one of X¹ and X² is H; the other of X¹ and X² is F; and R³ and R⁴ are each —CH₃ or R³ and R⁴ are taken, together with the atoms to which they are attached, to form cyclopropyl or cyclobutyl.

In some embodiments of a compound of formula (I-B), or a stereoisomer or tautomer thereof, or a pharmaceutically acceptable salt of any of the foregoing, Y¹ and Y² are each CH; X¹ and X² are independently H or halo; and R³ and R⁴ are each —CH₃ or R³ and R⁴ are taken, together with the atoms to which they are attached, to form cyclopropyl or cyclobutyl. In some embodiments, Y¹ and Y² are each CH; X¹ and X² are independently H or F; and R³ and R⁴ are each —CH₃ or R³ and R⁴ are taken, together with the atoms to which they are attached, to form cyclopropyl or cyclobutyl. In some embodiments, Y¹ and Y² are each CH; one of X¹ and X² is H; the other of X¹ and X² is halo; and R³ and R⁴ are each —CH₃ or R³ and R⁴ are taken, together with the atoms to which they are attached, to form cyclopropyl or cyclobutyl. In some embodiments, Y¹ and Y² are each CH; one of X¹ and X² is H; the other of X¹ and X² is F; and R³ and R⁴ are each —CH₃ or R³ and R⁴ are taken, together with the atoms to which they are attached, to form cyclopropyl or cyclobutyl.

In some embodiments of a compound of formula (I-B), or a stereoisomer or tautomer thereof, or a pharmaceutically acceptable salt of any of the foregoing, one of Y¹ and Y² is N and the other of Y¹ and Y² is CH; X¹ and X² are independently H or halo; and R³ and R⁴ are each —CH₃ or R³ and R⁴ are taken, together with the atoms to which they are attached, to form cyclopropyl or cyclobutyl. In some embodiments, one of Y¹ and Y² is N and the other of Y¹ and Y² is CH; X¹ and X² are independently H or F; and R³ and R⁴ are each —CH₃ or R³ and R⁴ are taken, together with the atoms to which they are attached, to form cyclopropyl or cyclobutyl. In some embodiments, one of Y¹ and Y² is N and the other of Y¹ and Y² is CH; one of X¹ and X² is H; the other of X¹ and X² is halo; and R³ and R⁴ are each —CH₃ or R³ and R⁴ are taken, together with the atoms to which they are attached, to form cyclopropyl or cyclobutyl. In some embodiments, one of Y¹ and Y² is N and the other of Y¹ and Y² is CH, one of X¹ and X² is H; the other of X¹ and X² is F; and R³ and R⁴ are each —CH₃ or R³ and R⁴ are taken, together with the atoms to which they are attached, to form cyclopropyl or cyclobutyl.

In some embodiments of a compound of formula (I-B), or a stereoisomer or tautomer thereof, or a pharmaceutically acceptable salt of any of the foregoing, X³ is H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

In some embodiments of a compound of formula (I-B), or a stereoisomer or tautomer thereof, or a pharmaceutically acceptable salt of any of the foregoing, X³ is H.

In some embodiments of a compound of formula (I-B), or a stereoisomer or tautomer thereof, or a pharmaceutically acceptable salt of any of the foregoing, X³ is —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

In some embodiments, one of X³ is selected from the group consisting of methyl, OH, Cl, —OCH₃, NH₂, —NH(CH₃),

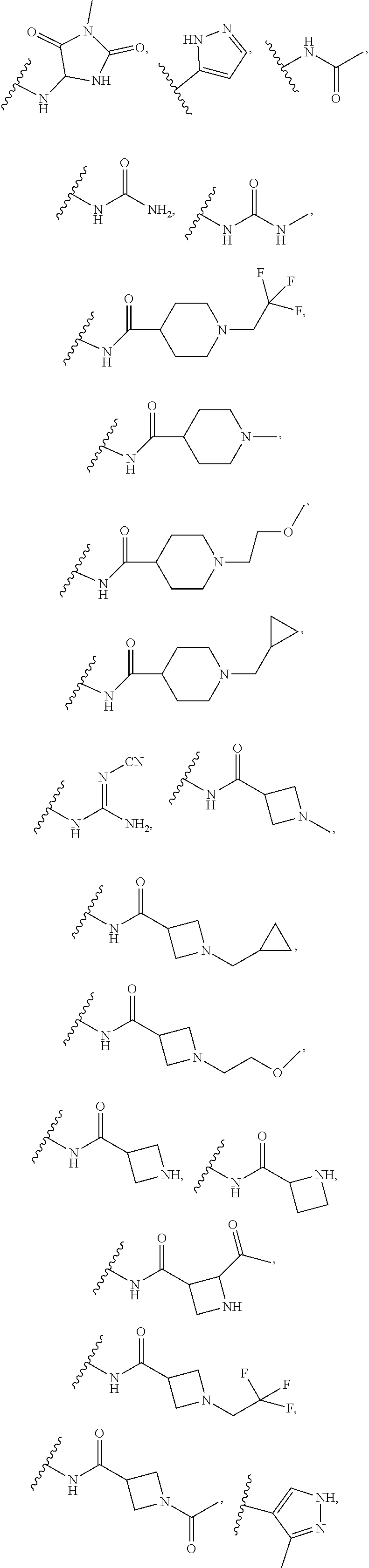

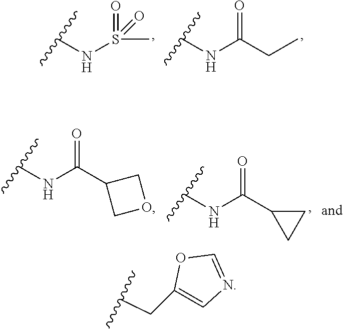

In some embodiments, provided herein is a compound of formula (I) or formula (I-B), or a stereoisomer or tautomer thereof, or a pharmaceutically acceptable salt thereof, wherein the compound is of formula (I-B1):

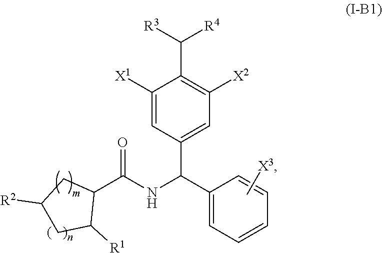

or a stereoisomer or tautomer thereof, or a pharmaceutically acceptable salt of any of the foregoing.

In some embodiments of a compound of formula (I-B1), or a stereoisomer or tautomer thereof, or a pharmaceutically acceptable salt of any of the foregoing, X¹ and X² are independently halo; X³ is C₁₋₃alkyl, or C₃₋₆cycloalkyl; and R³ and R⁴ are each —CH₃ or R³ and R⁴ are taken, together with the atoms to which they are attached, to form cyclopropyl or cyclobutyl.

In some embodiments, X¹ and X² are independently F; X³ is C₁₋₃alkyl, or C₃₋₆cycloalkyl; and R³ and R⁴ are each —CH₃ or R³ and R⁴ are taken, together with the atoms to which they are attached, to form cyclopropyl or cyclobutyl.

In some embodiments, provided is a compound of formula (I) or formula (I-B), or a stereoisomer or tautomer thereof, or a pharmaceutically acceptable salt of any of the foregoing, wherein the compound is a compound of formula (I-B2):

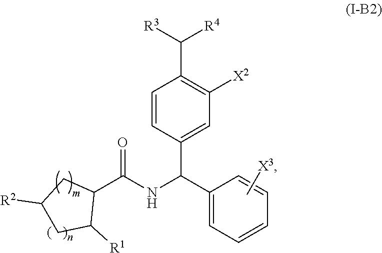

or a stereoisomer or tautomer thereof, or a pharmaceutically acceptable salt of any of the foregoing.

In some embodiments of a compound of formula (I-B2), or a stereoisomer or tautomer thereof, or a pharmaceutically acceptable salt of any of the foregoing, X² is halo; and R³ and R⁴ are each —CH₃ or R³ and R⁴ are taken, together with the atoms to which they are attached, to form cyclopropyl or cyclobutyl. In some embodiments, X² is F; and R³ and R⁴ are each —CH₃ or R³ and R⁴ are taken, together with the atoms to which they are attached, to form cyclopropyl or cyclobutyl.

In some embodiments, provided is a compound of formula (I) or formula (I-B), or a stereoisomer or tautomer thereof, or a pharmaceutically acceptable salt of any of the foregoing, wherein the compound is a compound of formula (I-B3):

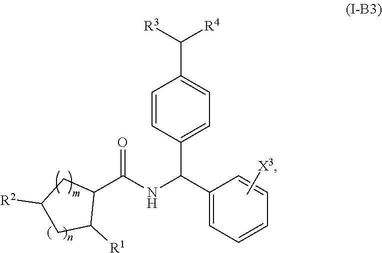

or a stereoisomer or tautomer thereof, or a pharmaceutically acceptable salt of any of the foregoing.

In some embodiments of a compound of formula (I-B3), or a stereoisomer or tautomer thereof, or a pharmaceutically acceptable salt of any of the foregoing, R³ and R⁴ are each —CH₃ or R³ and R⁴ are taken, together with the atoms to which they are attached, to form cyclopropyl or cyclobutyl.

In some embodiments, provided herein is a compound of formula (I) or formula (I-B), or a stereoisomer or tautomer thereof, or a pharmaceutically acceptable salt thereof, wherein the compound is of formula (I-B4):

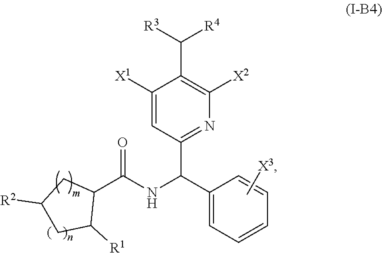

or a stereoisomer or tautomer thereof, or a pharmaceutically acceptable salt of any of the foregoing.

In some embodiments, provided is a compound of formula (I) or formula (I-B), or a stereoisomer or tautomer thereof, or a pharmaceutically acceptable salt of any of the foregoing, wherein the compound is a compound of formula (I-B5):

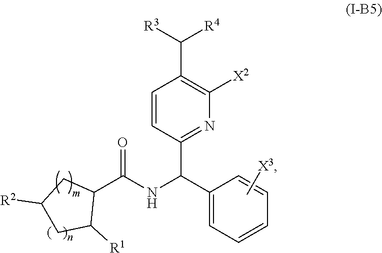

or a stereoisomer or tautomer thereof, or a pharmaceutically acceptable salt of any of the foregoing.

In some embodiments of a compound of formula (I-B5), or a stereoisomer or tautomer thereof, or a pharmaceutically acceptable salt of any of the foregoing, X² is halo; and X³ is C₁₋₆alkyl, or C₃₋₁₀cycloalkyl, wherein the C₃₋₁₀cycloalkyl of X³ is optionally substituted with one or more C₁₋₆alkyl. In some embodiments, X² is F; and X³ is C₁₋₃alkyl, or C₃₋₆cycloalkyl, wherein the C₃₋₆cycloalkyl of X³ is optionally substituted with one or more C₁₋₃alkyl.

In some embodiments, provided is a compound of formula (I) or formula (I-B), or a stereoisomer or tautomer thereof, or a pharmaceutically acceptable salt of any of the foregoing, wherein the compound is a compound of formula (I-B6):

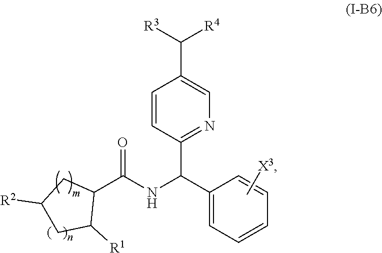

or a stereoisomer or tautomer thereof, or a pharmaceutically acceptable salt of any of the foregoing.

In some embodiments of a compound of formula (I-B6), or a stereoisomer or tautomer thereof, or a pharmaceutically acceptable salt of any of the foregoing, R³ and R⁴ are each —CH₃ or R³ and R⁴ are taken, together with the atoms to which they are attached, to form cyclopropyl or cyclobutyl.

In some embodiments, provided here is a compound of formula (I), or a stereoisomer or tautomer thereof, or a pharmaceutically acceptable salt of any of the foregoing, wherein the compound is a compound of formula (I-C):

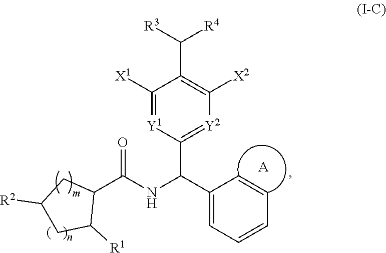

or a stereoisomer or tautomer thereof, or a pharmaceutically acceptable salt of any of the foregoing, wherein ring A is
3-9 membered heterocyclyl, and wherein the 3-9 membered heterocyclyl of ring A is optionally substituted with one or more oxo,
5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl.

In some embodiments, provided here is a compound of formula (I), or a stereoisomer or tautomer thereof, or a pharmaceutically acceptable salt of any of the foregoing, wherein the compound is a compound of formula (I-D):

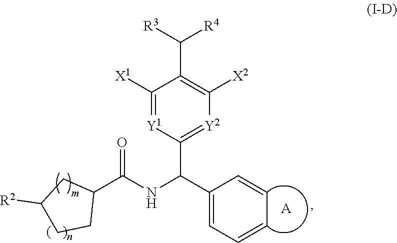

or a stereoisomer or tautomer thereof, or a pharmaceutically acceptable salt of any of the foregoing, wherein ring A is
3-9 membered heterocyclyl, and wherein the 3-9 membered heterocyclyl of ring A is optionally substituted with one or more oxo,
5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl.

In some embodiments of a compound of formula (I-C), (I-D), or a stereoisomer or tautomer thereof, or a pharmaceutically acceptable salt of any of the foregoing, ring A is 5-6 membered heterocyclyl, wherein the 5-6 membered heterocyclyl of ring A is optionally substituted with one or more oxo. In some embodiments, ring A is selected from the group consisting of

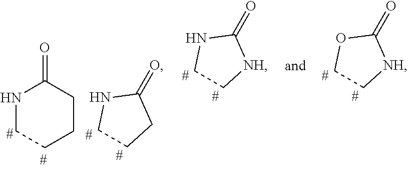

wherein # represents a point of attachment to the rest of the molecule.

In some embodiments of a compound of formula (I-C), or (I-D), or a stereoisomer or tautomer thereof, or a pharmaceutically acceptable salt of any of the foregoing, ring A is 5-8 membered heteroaryl, wherein the 5-8 membered heteroaryl of ring A comprises at least one annular N atom and is optionally substituted with one or more —NH₂, halo, C₁₋₆alkyl, or C₃₋₁₀cycloalkyl. In some embodiments, ring A is

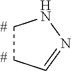

wherein # represents a point of attachment to the rest of the molecule.

In some embodiments, provided herein is a compound of formula (I), or a stereoisomer or tautomer thereof, or a pharmaceutically acceptable salt of any of the foregoing, wherein the compound is a compound of formula (I-E):

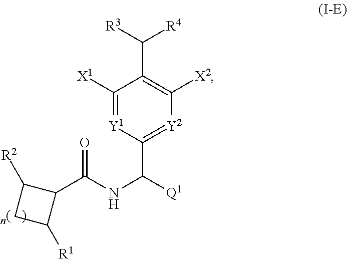

or a stereoisomer or tautomer thereof, or a pharmaceutically acceptable salt of any of the foregoing.

In some embodiments, provided herein is a compound of formula (I), or (I-E), or a stereoisomer or tautomer thereof, or a pharmaceutically acceptable salt of any of the foregoing, wherein the compound is a compound of formula (I-E1):

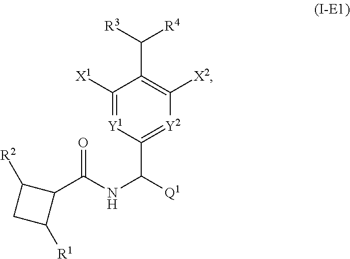

or a stereoisomer or tautomer thereof, or a pharmaceutically acceptable salt of any of the foregoing.

In some embodiments, provided herein is a compound of formula (I), or (I-E), or a stereoisomer or tautomer thereof, or a pharmaceutically acceptable salt of any of the foregoing, wherein the compound is a compound of formula (I-E2):

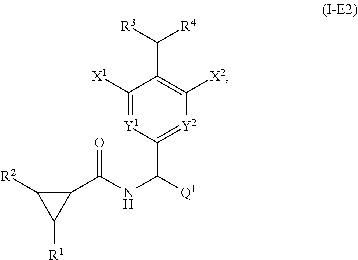

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is a compound of formula (I-F):

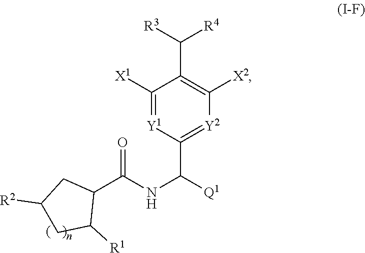

or a stereoisomer or tautomer thereof, or a pharmaceutically acceptable salt of any of the foregoing.

In some embodiments, provided herein is a compound of formula (I), or (I-F), or a stereoisomer or tautomer thereof, or a pharmaceutically acceptable salt of any of the foregoing, wherein the compound is a compound of formula (I-F1):

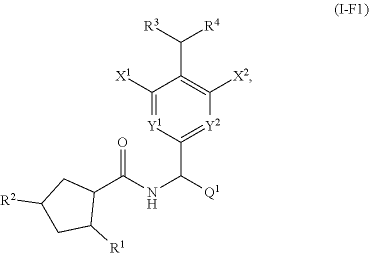

or a stereoisomer or tautomer thereof, or a pharmaceutically acceptable salt of any of the foregoing.

In some embodiments, provided herein is a compound of formula (I), or (I-F), or a stereoisomer or tautomer thereof, or a pharmaceutically acceptable salt of any of the foregoing, wherein the compound is a compound of formula (I-F2):

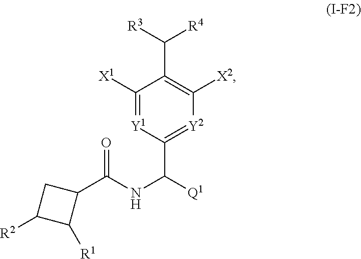

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is a compound of formula (I-G):

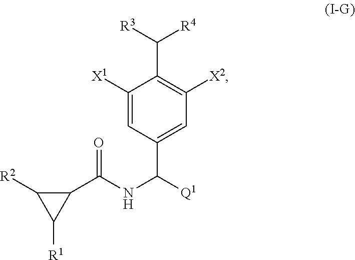

or a stereoisomer or tautomer thereof, or a pharmaceutically acceptable salt of any of the foregoing.

In some embodiments, provided is a compound of formula (I), or (I-G) or a stereoisomer or tautomer thereof, or a pharmaceutically acceptable salt of any of the foregoing, wherein the compound is a compound of formula (I-G1):

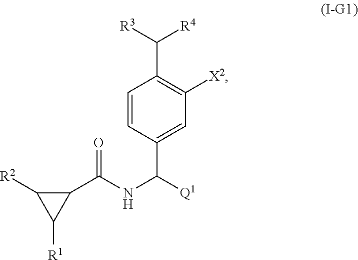

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G2):

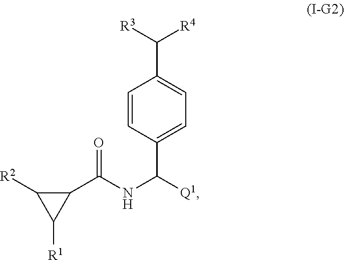

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G3):

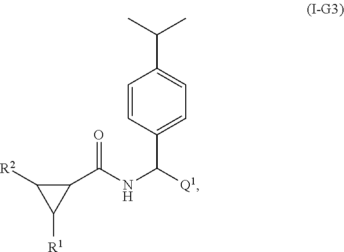

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G4):

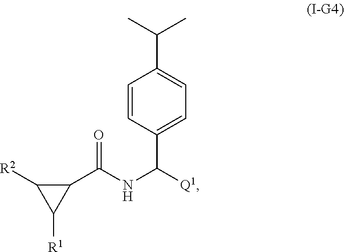

or a stereoisomer or tautomer thereof, or a pharmaceutically acceptable salt of any of the foregoing.

In some embodiments, provided is a compound of formula (I), or a stereoisomer or tautomer thereof, or a pharmaceutically acceptable salt of any of the foregoing, wherein the compound is a compound of formula (I-H):

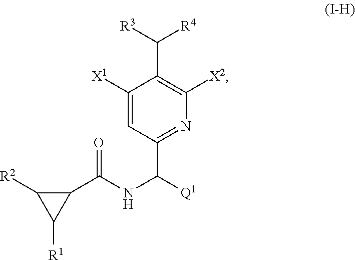

or a stereoisomer or tautomer thereof, or a pharmaceutically acceptable salt of any of the foregoing.

In some embodiments, provided is a compound of formula (I), or (I-H) or a stereoisomer or tautomer thereof, or a pharmaceutically acceptable salt of any of the foregoing, wherein the compound is a compound of formula (I-H1):

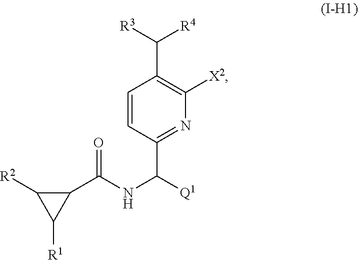

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H2):

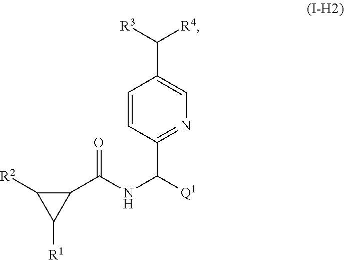

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H3):

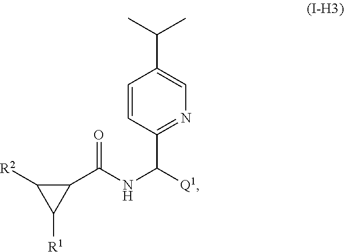

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H4):

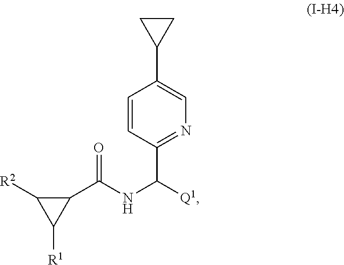

or a stereoisomer or tautomer thereof, or a pharmaceutically acceptable salt of any of the foregoing.

In some embodiments of a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1.

Compound Names included in Table 1 and for all intermediates and compounds were generated using ChemDraw® Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.1.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1

| Cmpd | | |
|---|---|---|
| No. | Structure | Cmpd Name |
| 1 | 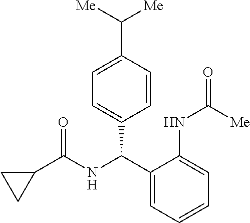 [SMILES: CC(=O)Nc1ccccc1[C@H](NC(=O)C1CC1)c1ccc(C(C)C)cc1] | (R)-N-((2-acetamidophenyl)(4- isopropylphenyl)methyl)cyclopropane- carboxamide |
| 2 | 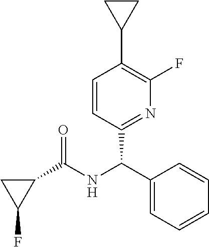 [SMILES: O=C(N[C@@H](c1ccccc1)c1ccc(C2CC2)c(F)n1)[C@H]1C[C@@H]1F] | (1R,2S)-N-((S)-(5-cyclopropyl-6- fluoropyridin-2-yl)(phenyl)methyl)- 2-fluorocyclopropane-1- carboxamide |
| 3 | 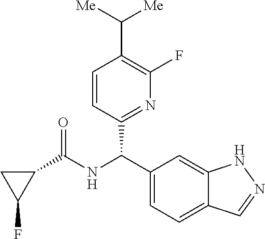 [SMILES: CC(C)c1ccc([C@@H](NC(=O)[C@H]2C[C@@H]2F)c2ccc3cn[nH]c3c2)nc1F] | (1R,2S)-2-fluoro-N-((S)-(6-fluoro-5- isopropylpyridin-2-yl)(1H-indazol- 6-yl)methyl)cyclopropane-1- carboxamide |
| 4 | 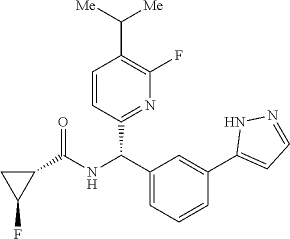 [SMILES: CC(C)c1ccc([C@@H](NC(=O)[C@H]2C[C@@H]2F)c2cccc(-c3ccn[nH]3)c2)nc1F] | (1R,2S)-N-((S)-(3-(1H-pyrazol-5- yl)phenyl)(6-fluoro-5- isopropylpyridin-2-yl)methyl)-2- fluorocyclopropane-1- carboxamide |
| 5 | 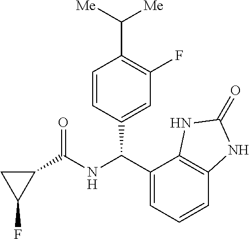 [SMILES: CC(C)c1ccc([C@@H](NC(=O)[C@H]2C[C@@H]2F)c2cccc3[nH]c(=O)[nH]c23)cc1F] | (1R,2S)-2-fluoro-N-((R)-(3-fluoro-4- isopropylphenyl)(2-oxo-2,3- dihydro-1H-benzo[d]imidazol-4- yl)methyl)cyclopropane-1- carboxamide |
| 6 | 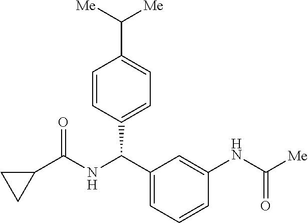 [SMILES: CC(=O)Nc1cccc([C@H](NC(=O)C2CC2)c2ccc(C(C)C)cc2)c1] | (R)-N-((3-acetamidophenyl)(4- isopropylphenyl)methyl) cyclopropanecarboxamide |
| 7 | 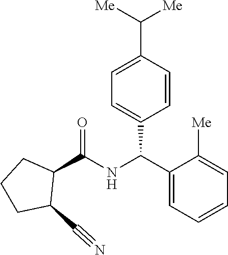 [SMILES: Cc1ccccc1[C@H](NC(=O)[C@@H]1CCC[C@@H]1C#N)c1ccc(C(C)C)cc1] | (1R,2S)-2-cyano-N-((R)-(4- isopropylphenyl)(o- tolyl)methyl)cyclopentane-1- carboxamide |
| 8 | 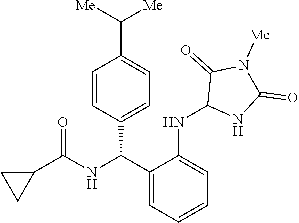 [SMILES: CC(C)c1ccc([C@@H](NC(=O)C2CC2)c2ccccc2NC2NC(=O)N(C)C2=O)cc1] | N-((1R)-(4-isopropylphenyl)(2-((1- methyl-2,5-dioxoimidazolidin-4- yl)amino)phenyl)methyl) cyclopropanecarboxamide |
| 9 | 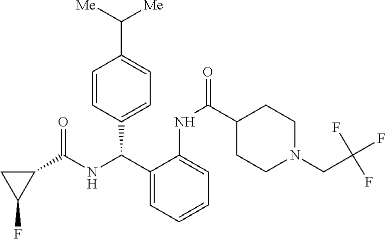 [SMILES: CC(C)c1ccc([C@@H](NC(=O)[C@H]2C[C@@H]2F)c2ccccc2NC(=O)C2CCN(CC(F)(F)F)CC2)cc1] | N-(2-((R)-((1R,2S)-2- fluorocyclopropane-1- carboxamido)(4- isopropylphenyl)methyl)phenyl)-1- (2,2,2-trifluoroethyl)piperidine-4- carboxamide |
| 10 | 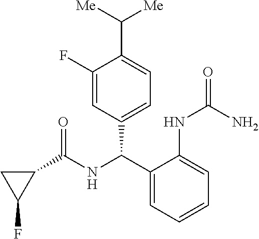 [SMILES: CC(C)c1ccc([C@@H](NC(=O)[C@H]2C[C@@H]2F)c2ccccc2NC(N)=O)cc1F] | (1R,2S)-2-fluoro-N-((R)-(3-fluoro-4- isopropylphenyl)(2- ureidophenyl)methyl)cyclopropane-1- carboxamide |
| 11 | 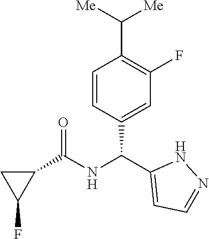 [SMILES: CC(C)c1ccc([C@@H](NC(=O)[C@H]2C[C@@H]2F)c2ccn[nH]2)cc1F] | (1R,2S)-2-fluoro-N-((R)-(3-fluoro-4- isopropylphenyl)(1H-pyrazol-5- yl)methyl)cyclopropane-1- carboxamide |
| 12 | 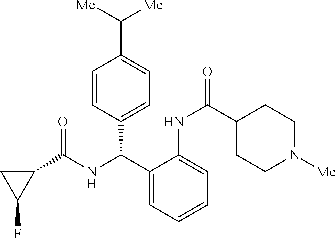 [SMILES: CC(C)c1ccc([C@@H](NC(=O)[C@H]2C[C@@H]2F)c2ccccc2NC(=O)C2CCN(C)CC2)cc1] | N-(2-((R)-((1R,2S)-2- fluorocyclopropane-1- carboxamido)(4- isopropylphenyl)methyl)phenyl)-1- methylpiperidine-4-carboxamide |
| 13 | 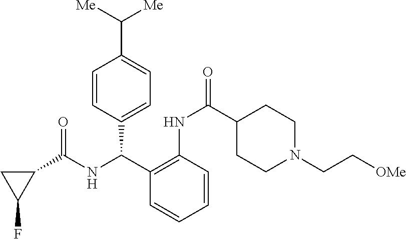 [SMILES: COCCN1CCC(C(=O)Nc2ccccc2[C@H](NC(=O)[C@H]2C[C@@H]2F)c2ccc(C(C)C)cc2)CC1] | N-(2-((R)-((1R,2S)-2- fluorocyclopropane-1- carboxamido)(4- isopropylphenyl)methyl)phenyl)-1- (2-methoxyethyl)piperidine-4- carboxamide |
| 14 | 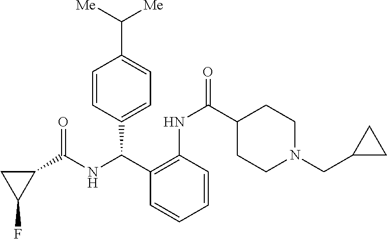 [SMILES: CC(C)c1ccc([C@@H](NC(=O)[C@H]2C[C@@H]2F)c2ccccc2NC(=O)C2CCN(CC3CC3)CC2)cc1] | 1-(cyclopropylmethyl)-N-(2-((R)- ((1R,2S)-2-fluorocyclopropane-1- carboxamido)(4- isopropylphenyl)methyl)phenyl) piperidine-4-carboxamide |
| 15 | 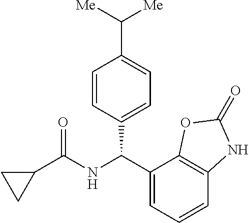 [SMILES: CC(C)c1ccc([C@@H](NC(=O)C2CC2)c2cccc3[nH]c(=O)oc23)cc1] | (R)-N-((4-isopropylphenyl)(2-oxo- 2,3-dihydrobenzo[d]oxazol-7- yl)methyl)cyclopropanecarboxamide |
| 16 | 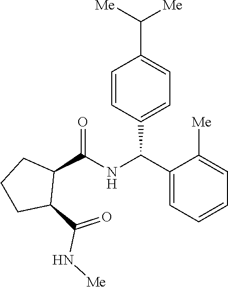 [SMILES: CNC(=O)[C@H]1CCC[C@H]1C(=O)N[C@H](c1ccc(C(C)C)cc1)c1ccccc1C] | (1R,2S)-N¹-((R)-(4- isopropylphenyl)(o-tolyl)methyl)- N²-methylcyclopentane-1,2- dicarboxamide |
| 17 | 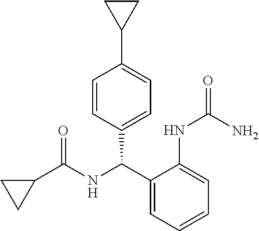 [SMILES: NC(=O)Nc1ccccc1[C@H](NC(=O)C1CC1)c1ccc(C2CC2)cc1] | (R)-N-((4-cyclopropylphenyl)(2- ureidophenyl)methyl)cyclopropane- carboxamide |
| 18 | 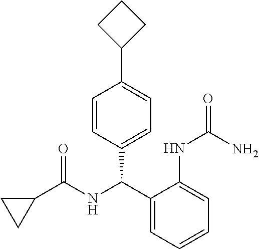 [SMILES: NC(=O)Nc1ccccc1[C@H](NC(=O)C1CC1)c1ccc(C2CCC2)cc1] | (R)-N-((4-cyclobutylphenyl)(2- ureidophenyl)methyl)cyclopropane- carboxamide |
| 19 | 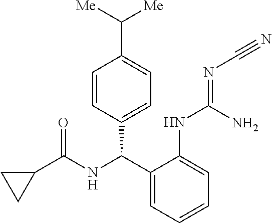 [SMILES: CC(C)c1ccc([C@@H](NC(=O)C2CC2)c2ccccc2N/C(N)=N/C#N)cc1] | (R,E)-N-((2-(2- cyanoguanidino)phenyl)(4- isopropylphenyl)methyl)cyclo- propanecarboxamide |
| 20 | 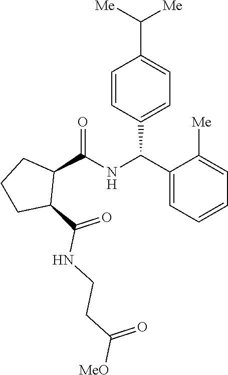 [SMILES: COC(=O)CCNC(=O)[C@H]1CCC[C@H]1C(=O)N[C@H](c1ccc(C(C)C)cc1)c1ccccc1C] | methyl 3-((1S,2R)-2-(((R)-(4- isopropylphenyl)(o- tolyl)methyl)carbamoyl)cyclo- pentane-1-carboxamido)propanoate |
| 21 | 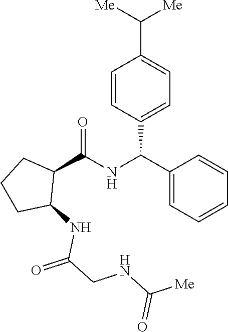 [SMILES: CC(=O)NCC(=O)N[C@H]1CCC[C@H]1C(=O)N[C@@H](c1ccccc1)c1ccc(C(C)C)cc1] | (1R,2S)-2-(2-acetamidoacetamido)- N-((S)-(4- isopropylphenyl)(phenyl)methyl) cyclopentane-1-carboxamide |
| 22 | 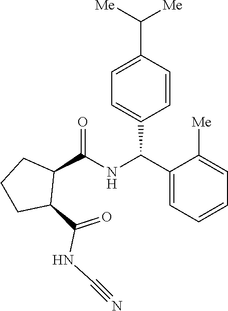 [SMILES: Cc1ccccc1[C@H](NC(=O)[C@@H]1CCC[C@@H]1C(=O)NC#N)c1ccc(C(C)C)cc1] | (1S,2R)-N¹-cyano-N²-((R)-(4- isopropylphenyl)(o- tolyl)methyl)cyclopentane-1,2- dicarboxamide |
| 23 | 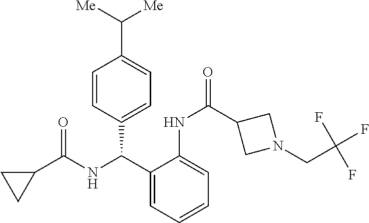 [SMILES: CC(C)c1ccc([C@@H](NC(=O)C2CC2)c2ccccc2NC(=O)C2CN(CC(F)(F)F)C2)cc1] | (R)-N-(2- (cyclopropanecarboxamido(4- isopropylphenyl)methyl)phenyl)-1- (2,2,2-trifluoroethyl)azetidine-3- carboxamide |
| 24 | 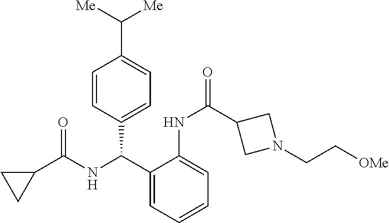 [SMILES: COCCN1CC(C(=O)Nc2ccccc2[C@H](NC(=O)C2CC2)c2ccc(C(C)C)cc2)C1] | (R)-N-(2- (cyclopropanecarboxamido(4- isopropylphenyl)methyl)phenyl)-1- (2-methoxyethyl)azetidine-3- carboxamide |
| 25 | 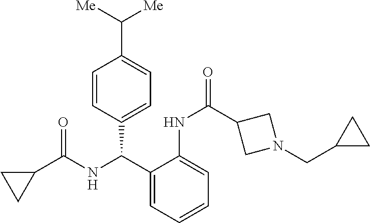 [SMILES: CC(C)c1ccc([C@@H](NC(=O)C2CC2)c2ccccc2NC(=O)C2CN(CC3CC3)C2)cc1] | (R)-N-(2- (cyclopropanecarboxamido(4- isopropylphenyl)methyl)phenyl)-1- (cyclopropylmethyl)azetidine-3- carboxamide |
| 26 | 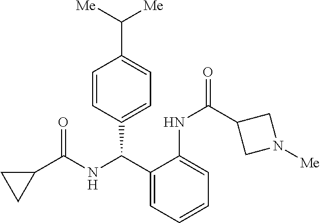 [SMILES: CC(C)c1ccc([C@@H](NC(=O)C2CC2)c2ccccc2NC(=O)C2CN(C)C2)cc1] | (R)-N-(2- (cyclopropanecarboxamido(4- isopropylphenyl)methyl)phenyl)-1- methylazetidine-3-carboxamide |
| 27 | 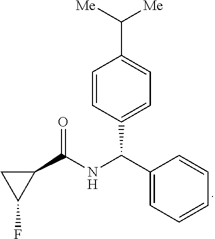 [SMILES: CC(C)c1ccc([C@@H](NC(=O)[C@@H]2C[C@H]2F)c2ccccc2)cc1] | (1S,2R)-2-fluoro-N-((S)-(4- isopropylphenyl)(phenyl)methyl) cyclopropane-1-carboxamide |
| 28 | 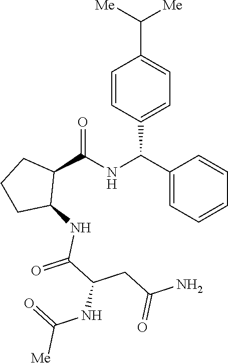 [SMILES: CC(=O)N[C@@H](CC(N)=O)C(=O)N[C@H]1CCC[C@H]1C(=O)N[C@@H](c1ccccc1)c1ccc(C(C)C)cc1] | (S)-2-acetamido-N¹-((1S,2R)-2-(((S)- (4- isopropylphenyl)(phenyl)methyl) carbamoyl)cyclopentyl)succinamide |
| 29 | 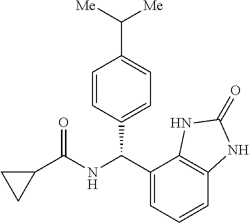 [SMILES: CC(C)c1ccc([C@@H](NC(=O)C2CC2)c2cccc3[nH]c(=O)[nH]c23)cc1] | (R)-N-((4-isopropylphenyl)(2-oxo- 2,3-dihydro-1H-benzo[d]imidazol-4- yl)methyl)cyclopropanecarboxamide |
| 30 | 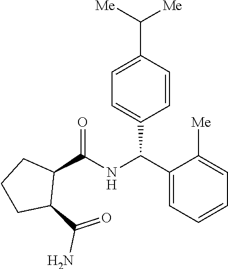 [SMILES: Cc1ccccc1[C@H](NC(=O)[C@@H]1CCC[C@@H]1C(N)=O)c1ccc(C(C)C)cc1] | (1R,2S)-N¹-((R)-(4- isopropylphenyl)(o- tolyl)methyl)cyclopentane-1,2- dicarboxamide |
| 31 | 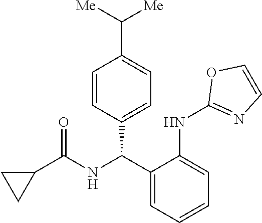 [SMILES: CC(C)c1ccc([C@@H](NC(=O)C2CC2)c2ccccc2Nc2ncco2)cc1] | (R)-N-((4-isopropylphenyl)(2- (oxazol-2- ylamino)phenyl)methyl)cyclopropane- carboxamide |
| 32 | 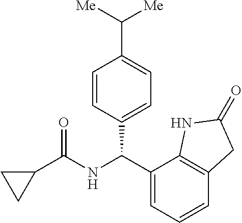 [SMILES: CC(C)c1ccc([C@@H](NC(=O)C2CC2)c2cccc3c2NC(=O)C3)cc1] | (R)-N-((4-isopropylphenyl)(2- oxoindolin-7- yl)methyl)cyclopropanecarboxamide |
| 33 | 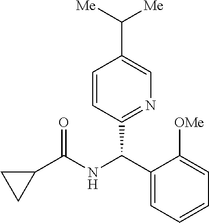 [SMILES: COc1ccccc1[C@H](NC(=O)C1CC1)c1ccc(C(C)C)cn1] | (S)-N-((5-isopropylpyridin-2-yl)(2- methoxyphenyl)methyl)cyclopropane- carboxamide |
| 34 | 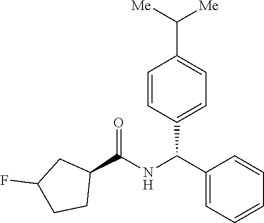 [SMILES: CC(C)c1ccc([C@@H](NC(=O)[C@H]2CCC(F)C2)c2ccccc2)cc1] | (1S)-3-fluoro-N-((S)-(4- isopropylphenyl)(phenyl)methyl) cyclopentane-1-carboxamide |
| 35 | 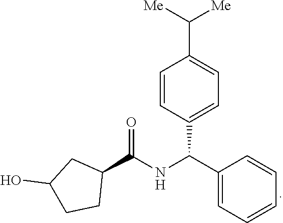 [SMILES: CC(C)c1ccc([C@@H](NC(=O)[C@H]2CCC(O)C2)c2ccccc2)cc1] | (1S)-3-hydroxy-N-((S)-(4- isopropylphenyl)(phenyl)methyl) cyclopentane-1-carboxamide |
| 36 | 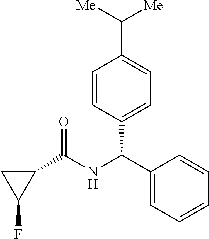 [SMILES: CC(C)c1ccc([C@@H](NC(=O)[C@H]2C[C@@H]2F)c2ccccc2)cc1] | (1R,2S)-2-fluoro-N-((S)-(4- isopropylphenyl)(phenyl)methyl) cyclopropane-1-carboxamide |
| 37 | 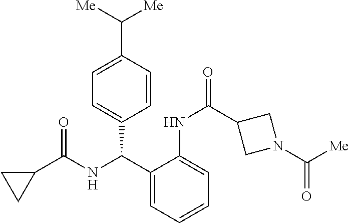 [SMILES: CC(=O)N1CC(C(=O)Nc2ccccc2[C@H](NC(=O)C2CC2)c2ccc(C(C)C)cc2)C1] | (R)-1-acetyl-N-(2- (cyclopropanecarboxamido(4- isopropylphenyl)methyl)phenyl) azetidine-3-carboxamide |
| 38 | 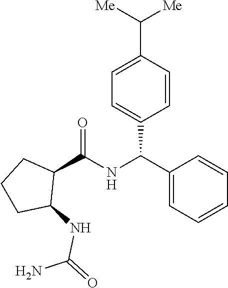 [SMILES: CC(C)c1ccc([C@@H](NC(=O)[C@@H]2CCC[C@@H]2NC(N)=O)c2ccccc2)cc1] | (1R,2S)-N-((S)-(4- isopropylphenyl)(phenyl)methyl)-2- ureidocyclopentane-1-carboxamide |
| 39 | 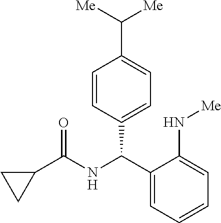 [SMILES: CNc1ccccc1[C@H](NC(=O)C1CC1)c1ccc(C(C)C)cc1] | (R)-N-((4-isopropylphenyl)(2- (methylamino)phenyl)methyl) cyclopropanecarboxamide |
| 40 | 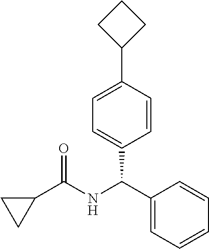 [SMILES: O=C(N[C@@H](c1ccccc1)c1ccc(C2CCC2)cc1)C1CC1] | (S)-N-((4- cyclobutylphenyl)(phenyl)methyl) cyclopropanecarboxamide |
| 41 | 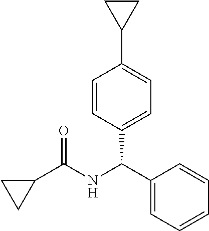 [SMILES: O=C(N[C@@H](c1ccccc1)c1ccc(C2CC2)cc1)C1CC1] | (S)-N-((4- cyclopropylphenyl)(phenyl)methyl) cyclopropanecarboxamide |
| 42 | 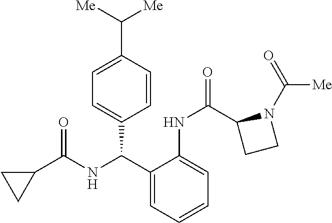 [SMILES: CC(=O)N1CC[C@H]1C(=O)Nc1ccccc1[C@H](NC(=O)C1CC1)c1ccc(C(C)C)cc1] | (S)-1-acetyl-N-(2-((R)- cyclopropanecarboxamido(4- isopropylphenyl)methyl)phenyl) azetidine-2-carboxamide |
| 43 | 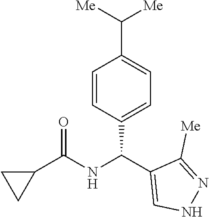 [SMILES: Cc1n[nH]cc1[C@H](NC(=O)C1CC1)c1ccc(C(C)C)cc1] | (R)-N-((4-isopropylphenyl)(3- methyl-1H-pyrazol-4- yl)methyl)cyclopropanecarboxamide |
| 44 | 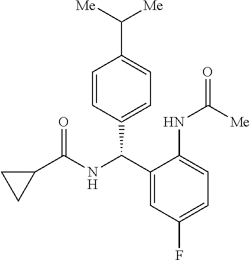 [SMILES: CC(=O)Nc1ccc(F)cc1[C@H](NC(=O)C1CC1)c1ccc(C(C)C)cc1] | (R)-N-((2-acetamido-5- fluorophenyl)(4- isopropylphenyl)methyl) cyclopropanecarboxamide |
| 45 | 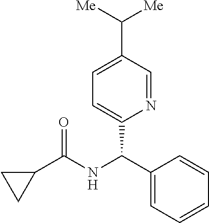 [SMILES: CC(C)c1ccc([C@@H](NC(=O)C2CC2)c2ccccc2)nc1] | (S)-N-((5-isopropylpyridin-2- yl)(phenyl)methyl) cyclopropanecarboxamide |
| 46 | 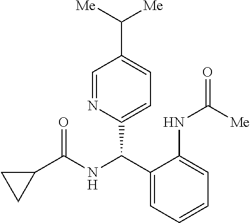 [SMILES: CC(=O)Nc1ccccc1[C@H](NC(=O)C1CC1)c1ccc(C(C)C)cn1] | (S)-N-((2-acetamidophenyl)(5- isopropylpyridin-2- yl)methyl)cyclopropanecarboxamide |
| 47 | 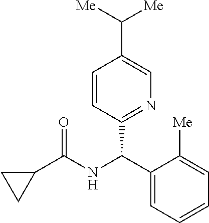 [SMILES: Cc1ccccc1[C@H](NC(=O)C1CC1)c1ccc(C(C)C)cn1] | (S)-N-((5-isopropylpyridin-2-yl)(o- tolyl)methyl)cyclopropanecarboxamide |
| 48 | 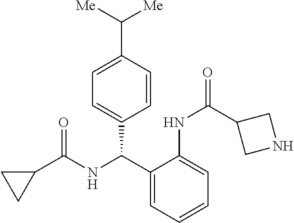 [SMILES: CC(C)c1ccc([C@@H](NC(=O)C2CC2)c2ccccc2NC(=O)C2CNC2)cc1] | (R)-N-(2- (cyclopropanecarboxamido(4- isopropylphenyl)methyl)phenyl) azetidine-3-carboxamide |
| 49 | 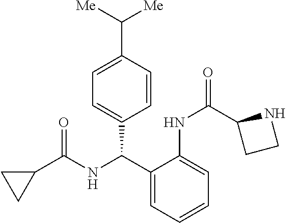 [SMILES: CC(C)c1ccc([C@@H](NC(=O)C2CC2)c2ccccc2NC(=O)[C@@H]2CCN2)cc1] | (S)-N-(2-((R)- cyclopropanecarboxamido(4- isopropylphenyl)methyl)phenyl) azetidine-2-carboxamide |
| 50 | 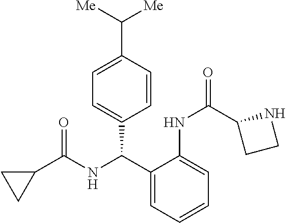 [SMILES: CC(C)c1ccc([C@@H](NC(=O)C2CC2)c2ccccc2NC(=O)[C@H]2CCN2)cc1] | (R)-N-(2-((R)- cyclopropanecarboxamido(4- isopropylphenyl)methyl)phenyl) azetidine-2-carboxamide |
| 51 | 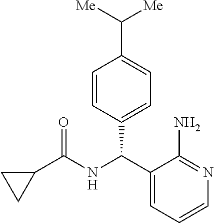 [SMILES: CC(C)c1ccc([C@@H](NC(=O)C2CC2)c2cccnc2N)cc1] | (R)-N-((2-aminopyridin-3-yl)(4- isopropylphenyl)methyl) cyclopropanecarboxamide |
| 52 | 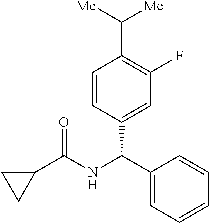 [SMILES: CC(C)c1ccc([C@@H](NC(=O)C2CC2)c2ccccc2)cc1F] | (S)-N-((3-fluoro-4- isopropylphenyl)(phenyl)methyl) cyclopropanecarboxamide |
| 53 | 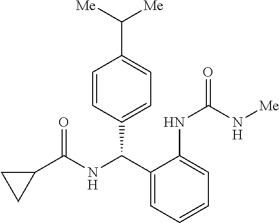 [SMILES: CNC(=O)Nc1ccccc1[C@H](NC(=O)C1CC1)c1ccc(C(C)C)cc1] | (R)-N-((4-isopropylphenyl)(2-(3- methylureido)phenyl)methyl) cyclopropanecarboxamide |
| 54 | 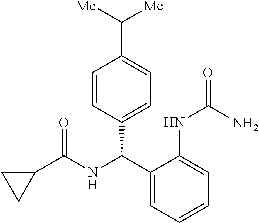 [SMILES: CC(C)c1ccc([C@@H](NC(=O)C2CC2)c2ccccc2NC(N)=O)cc1] | (R)-N-((4-isopropylphenyl)(2- ureidophenyl)methyl) cyclopropanecarboxamide |
| 55 | 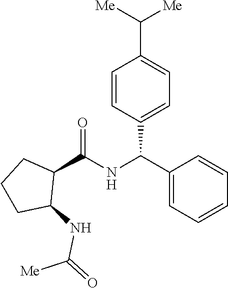 [SMILES: CC(=O)N[C@H]1CCC[C@H]1C(=O)N[C@@H](c1ccccc1)c1ccc(C(C)C)cc1] | (1R,2S)-2-acetamido-N-((S)-(4- isopropylphenyl)(phenyl)methyl) cyclopentane-1-carboxamide |
| 56 | 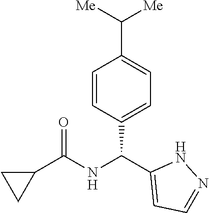 [SMILES: CC(C)c1ccc([C@@H](NC(=O)C2CC2)c2ccn[nH]2)cc1] | (R)-N-((4-isopropylphenyl)(1H- pyrazol-5- yl)methyl)cyclopropanecarboxamide |
| 57 | 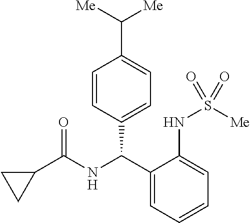 [SMILES: CC(C)c1ccc([C@@H](NC(=O)C2CC2)c2ccccc2NS(C)(=O)=O)cc1] | (R)-N-((4-isopropylphenyl)(2- (methylsulfonamido)phenyl)methyl) cyclopropanecarboxamide |
| 58 | 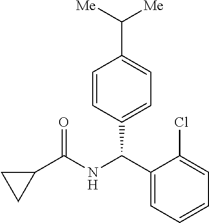 [SMILES: CC(C)c1ccc([C@@H](NC(=O)C2CC2)c2ccccc2Cl)cc1] | (R)-N-((2-chlorophenyl)(4- isopropylphenyl)methyl) cyclopropanecarboxamide |
| 59 | 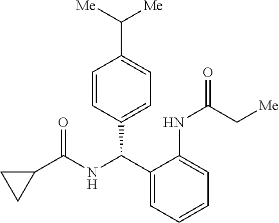 [SMILES: CCC(=O)Nc1ccccc1[C@H](NC(=O)C1CC1)c1ccc(C(C)C)cc1] | (R)-N-((4-isopropylphenyl)(2- propionamidophenyl)methyl) cyclopropanecarboxamide |
| 60 | 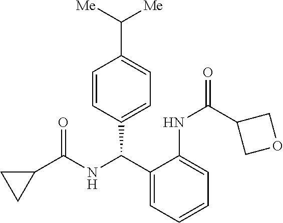 [SMILES: CC(C)c1ccc([C@@H](NC(=O)C2CC2)c2ccccc2NC(=O)C2COC2)cc1] | (R)-N-(2- (cyclopropanecarboxamido(4- isopropylphenyl)methyl)phenyl) oxetane-3-carboxamide |
| 61 | 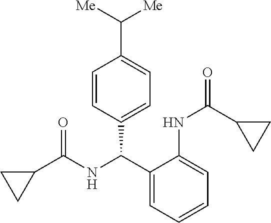 [SMILES: CC(C)c1ccc([C@@H](NC(=O)C2CC2)c2ccccc2NC(=O)C2CC2)cc1] | (R)-N-(2- (cyclopropanecarboxamido(4- isopropylphenyl)methyl)phenyl) cyclopropanecarboxamide |
| 62 | 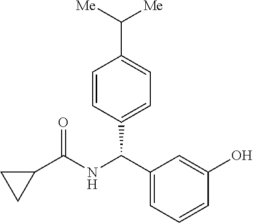 [SMILES: CC(C)c1ccc([C@@H](NC(=O)C2CC2)c2cccc(O)c2)cc1] | (R)-N-((3-hydroxyphenyl)(4- isopropylphenyl)methyl) cyclopropanecarboxamide |
| 63 | 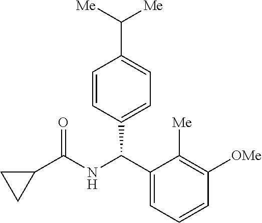 [SMILES: COc1cccc([C@H](NC(=O)C2CC2)c2ccc(C(C)C)cc2)c1C] | (R)-N-((4-isopropylphenyl)(3- methoxy-2- methylphenyl)methyl) cyclopropanecarboxamide |
| 64 | 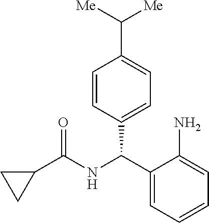 [SMILES: CC(C)c1ccc([C@@H](NC(=O)C2CC2)c2ccccc2N)cc1] | (R)-N-((2-aminophenyl)(4- isopropylphenyl)methyl) cyclopropanecarboxamide |
| 65 | 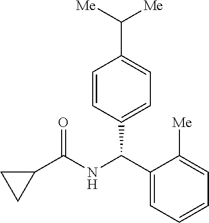 [SMILES: Cc1ccccc1[C@H](NC(=O)C1CC1)c1ccc(C(C)C)cc1] | (R)-N-((4-isopropylphenyl)(o- tolyl)methyl) cyclopropanecarboxamide |
| 66 | 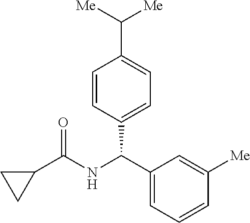 [SMILES: Cc1cccc([C@H](NC(=O)C2CC2)c2ccc(C(C)C)cc2)c1] | (R)-N-((4-isopropylphenyl)(m- tolyl)methyl)cyclopropanecarboxamide |
| 67 | 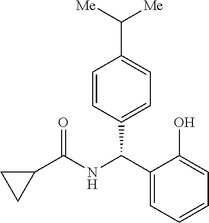 [SMILES: CC(C)c1ccc([C@@H](NC(=O)C2CC2)c2ccccc2O)cc1] | (R)-N-((2-hydroxyphenyl)(4- isopropylphenyl)methyl) cyclopropanecarboxamide |
| 68 | 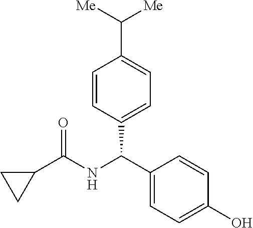 [SMILES: CC(C)c1ccc([C@@H](NC(=O)C2CC2)c2ccc(O)cc2)cc1] | (R)-N-((4-hydroxyphenyl)(4- isopropylphenyl)methyl) cyclopropanecarboxamide |
| 69 | 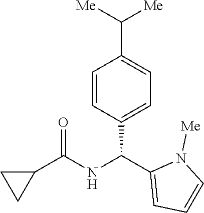 [SMILES: CC(C)c1ccc([C@@H](NC(=O)C2CC2)c2cccn2C)cc1] | (R)-N-((4-isopropylphenyl)(1- methyl-1H-pyrazol-5- yl)methyl)cyclopropanecarboxamide |
| 70 | 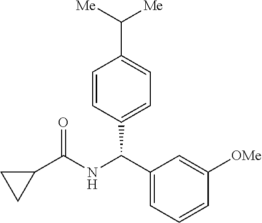 [SMILES: COc1cccc([C@H](NC(=O)C2CC2)c2ccc(C(C)C)cc2)c1] | (R)-N-((4-isopropylphenyl)(3- methoxyphenyl)methyl) cyclopropanecarboxamide |
| 71 | 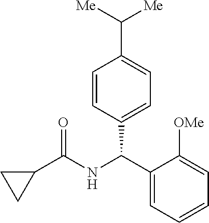 [SMILES: COc1ccccc1[C@H](NC(=O)C1CC1)c1ccc(C(C)C)cc1] | (R)-N-((4-isopropylphenyl)(2- methoxyphenyl)methyl) cyclopropanecarboxamide |
| 72 | 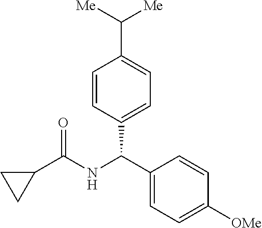 [SMILES: COc1ccc([C@H](NC(=O)C2CC2)c2ccc(C(C)C)cc2)cc1] | (R)-N-((4-isopropylphenyl)(4- methoxyphenyl)methyl) cyclopropanecarboxamide |
| 73 | 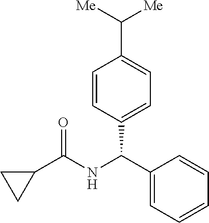 [SMILES: CC(C)c1ccc([C@@H](NC(=O)C2CC2)c2ccccc2)cc1] | (S)-N-((4- isopropylphenyl)(phenyl)methyl) cyclopropanecarboxamide |

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
N-{phenyl[4-(propan-2-yl)phenyl]methyl}cyclopropanecarboxamide;
N-[(4-methoxyphenyl)[4-(propan-2-yl)phenyl]methyl]cyclopropanecarboxamide;
N-[(2-methoxyphenyl)[4-(propan-2-yl)phenyl]methyl]cyclopropanecarboxamide;
N-[(3-methoxyphenyl)[4-(propan-2-yl)phenyl]methyl]cyclopropanecarboxamide;
N-[(1-methyl-1H-pyrazol-5-yl)[4-(propan-2-yl)phenyl]methyl]cyclopropanecarboxamide;
N-[(4-hydroxyphenyl)[4-(propan-2-yl)phenyl]methyl]cyclopropanecarboxamide;
N-[(2-hydroxyphenyl)[4-(propan-2-yl)phenyl]methyl]cyclopropanecarboxamide;
N-[(3-methylphenyl)[4-(propan-2-yl)phenyl]methyl]cyclopropanecarboxamide;
N-[(2-methylphenyl)[4-(propan-2-yl)phenyl]methyl]cyclopropanecarboxamide;
N-[(2-acetamidophenyl)[4-(propan-2-yl)phenyl]methyl]cyclopropanecarboxamide;
N-[(2-aminophenyl)[4-(propan-2-yl)phenyl]methyl]cyclopropanecarboxamide;
N-[(3-methoxy-2-methylphenyl)[4-(propan-2-yl)phenyl]methyl]cyclopropanecarboxamide;
N-[(3-hydroxyphenyl)[4-(propan-2-yl)phenyl]methyl]cyclopropanecarboxamide;
N-[(2-cyclopropaneamidophenyl)[4-(propan-2-yl)phenyl]methyl]cyclopropanecarboxamide;
N-{2-[(cyclopropylformamido)[4-(propan-2-yl)phenyl]methyl]phenyl}oxetane-3-carboxamide;
N-{[4-(propan-2-yl)phenyl](2-propanamidophenyl)methyl}cyclopropanecarboxamide;
N-[(2-chlorophenyl)[4-(propan-2-yl)phenyl]methyl]cyclopropanecarboxamide;
N-[(2-methanesulfonamidophenyl)[4-(propan-2-yl)phenyl]methyl]cyclopropanecarboxamide;
N-{[4-(propan-2-yl)phenyl](1H-pyrazol-5-yl)methyl}cyclopropanecarboxamide;
2-acetamido-N-{phenyl[4-(propan-2-yl)phenyl]methyl}cyclopentane-1-carboxamide;
N-{[2-(carbamoylamino)phenyl][4-(propan-2-yl)phenyl]methyl}cyclopropanecarboxamide;
N-({2-[(methylcarbamoyl)amino]phenyl}[4-(propan-2-yl)phenyl]methyl)cyclopropanecarboxamide;
N-[(2-aminopyridin-3-yl)[4-(propan-2-yl)phenyl]methyl]cyclopropanecarboxamide;
N-{[3-fluoro-4-(propan-2-yl)phenyl](phenyl)methyl}cyclopropanecarboxamide;
N-{2-[(cyclopropylformamido)[4-(propan-2-yl)phenyl]methyl]phenyl}azetidine-2-carboxamide;
N-[(2-methylphenyl)[5-(propan-2-yl)pyridin-2-yl]methyl]cyclopropanecarboxamide;
N-{2-[(cyclopropylformamido)[4-(propan-2-yl)phenyl]methyl]phenyl}azetidine-3-carboxamide;
N-[(4-cyclopropylphenyl)(phenyl)methyl]cyclopropanecarboxamide;
N-{phenyl[5-(propan-2-yl)pyridin-2-yl]methyl}cyclopropanecarboxamide;
N-[(4-cyclobutylphenyl)(phenyl)methyl]cyclopropanecarboxamide;
N-[(2-acetamido-5-fluorophenyl)[4-(propan-2-yl)phenyl]methyl]cyclopropanecarboxamide;
N-[(3-methyl-1H-pyrazol-4-yl)[4-(propan-2-yl)phenyl]methyl]cyclopropanecarboxamide;
N-{[2-(methylamino)phenyl][4-(propan-2-yl)phenyl]methyl}cyclopropanecarboxamide;
1-acetyl-N-{2-[(cyclopropylformamido)[4-(propan-2-yl)phenyl]methyl]phenyl}azetidine-2-carboxamide;
2-(carbamoylamino)-N-{phenyl[4-(propan-2-yl)phenyl]methyl}cyclopentane-1-carboxamide;
N-[(2-methoxyphenyl)[5-(propan-2-yl)pyridin-2-yl]methyl]cyclopropanecarboxamide;
1-acetyl-N-{2-[(cyclopropylformamido)[4-(propan-2-yl)phenyl]methyl]phenyl}azetidine-3-carboxamide;
2-fluoro-N-{phenyl[4-(propan-2-yl)phenyl]methyl}cyclopropane-1-carboxamide;
3-hydroxy-N-{phenyl[4-(propan-2-yl)phenyl]methyl}cyclopentane-1-carboxamide;
3-fluoro-N-{phenyl[4-(propan-2-yl)phenyl]methyl}cyclopentane-1-carboxamide;
N-[(2-oxo-2,3-dihydro-1H-indol-7-yl)[4-(propan-2-yl)phenyl]methyl]cyclopropanecarboxamide;
N-({2-[(1,3-oxazol-2-yl)amino]phenyl}[4-(propan-2-yl)phenyl]methyl)cyclopropanecarboxamide;
N1-[(2-methylphenyl)[4-(propan-2-yl)phenyl]methyl]cyclopentane-1,2-dicarboxamide;
N-[(2-oxo-2,3-dihydro-1H-1,3-benzodiazol-4-yl)[4-(propan-2-yl)phenyl]methyl]cyclopropanecarboxamide;
2-acetamido-N-[2-({phenyl[4-(propan-2-yl)phenyl]methyl}carbamoyl)cyclopentyl]butanediamide;
2-fluoro-N-{phenyl[4-(propan-2-yl)phenyl]methyl}cyclopropane-1-carboxamide;
N-{2-[(cyclopropylformamido)[4-(propan-2-yl)phenyl]methyl]phenyl}-1-methylazetidine-3-carboxamide;
N-{2-[(cyclopropylformamido)[4-(propan-2-yl)phenyl]methyl]phenyl}-1-(cyclopropylmethyl)azetidine-3-carboxamide;
N-{2-[(cyclopropylformamido)[4-(propan-2-yl)phenyl]methyl]phenyl}-1-(2-methoxyethyl)azetidine-3-carboxamide;
N-{2-[(cyclopropylformamido)[4-(propan-2-yl)phenyl]methyl]phenyl}-1-(2,2,2-trifluoroethyl)azetidine-3-carboxamide;
N1-cyano-N2-[(2-methylphenyl)[4-(propan-2-yl)phenyl]methyl]cyclopentane-1,2-dicarboxamide;
2-(2-acetamidoacetamido)-N-{phenyl[4-(propan-2-yl)phenyl]methyl}cyclopentane-1-carboxamide;
methyl 3-[(2-{[(2-methylphenyl)[4-(propan-2-yl)phenyl]methyl]carbamoyl}cyclopentyl)formamido]propanoate;
N-{[2-(N″-cyanocarbamimidamido)phenyl][4-(propan-2-yl)phenyl]methyl}cyclopropanecarboxamide;
N-{[2-(carbamoylamino)phenyl](4-cyclobutylphenyl)methyl}cyclopropanecarboxamide;
N1-methyl-N2-[(2-methylphenyl)[4-(propan-2-yl)phenyl]methyl]cyclopentane-1,2-dicarboxamide;
N-{[2-(carbamoylamino)phenyl](4-cyclopropylphenyl)methyl}cyclopropanecarboxamide;
N-[(2-oxo-2,3-dihydro-1,3-benzoxazol-7-yl)[4-(propan-2-yl)phenyl]methyl]cyclopropanecarboxamide;
1-(cyclopropylmethyl)-N-(2-{[(2-fluorocyclopropyl)formamido][4-(propan-2-yl)phenyl]methyl}phenyl)piperidine-4-carboxamide;
N-(2-{[(2-fluorocyclopropyl)formamido][4-(propan-2-yl)phenyl]methyl}phenyl)-1-(2-methoxyethyl)piperidine-4-carboxamide;
N-(2-{[(2-fluorocyclopropyl)formamido][4-(propan-2-yl)phenyl]methyl}phenyl)-1-methylpiperidine-4-carboxamide;
2-fluoro-N-{[3-fluoro-4-(propan-2-yl)phenyl](1H-pyrazol-5-yl)methyl}cyclopropane-1-carboxamide;
N-{[2-(carbamoylamino)phenyl][3-fluoro-4-(propan-2-yl)phenyl]methyl}-2-fluorocyclopropane-1-carboxamide;
N-(2-{[(2-fluorocyclopropyl)formamido][4-(propan-2-yl)phenyl]methyl}phenyl)-1-(2,2,2-trifluoroethyl)piperidine-4-carboxamide;
N-({2-[(1-methyl-2,5-dioxoimidazolidin-4-yl)amino]phenyl}[4-(propan-2-yl)phenyl]methyl)cyclopropanecarboxamide;
2-cyano-N-[(2-methylphenyl)[4-(propan-2-yl)phenyl]methyl]cyclopentane-1-carboxamide;
N-[(3-acetamidophenyl)[4-(propan-2-yl)phenyl]methyl]cyclopropanecarboxamide;
2-fluoro-N-{[3-fluoro-4-(propan-2-yl)phenyl](2-oxo-2,3-dihydro-1H-1,3-benzodiazol-4-yl)methyl}cyclopropane-1-carboxamide;
2-fluoro-N-{[6-fluoro-5-(propan-2-yl)pyridin-2-yl][3-(1H-pyrazol-5-yl)phenyl]methyl}cyclopropane-1-carboxamide;
2-fluoro-N-{[6-fluoro-5-(propan-2-yl)pyridin-2-yl](1H-indazol-6-yl)methyl}cyclopropane-1-carboxamide; and
N-[(5-cyclopropyl-6-fluoropyridin-2-yl)(phenyl)methyl]-2-fluorocyclopropane-1-carboxamide;
   or a tautomer thereof, or a pharmaceutically acceptable salt of any of the foregoing.

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
(R)—N-((2-acetamidophenyl)(4-isopropylphenyl)methyl)cyclopropanecarboxamide;
(1R,2S)—N—((S)-(5-cyclopropyl-6-fluoropyridin-2-yl)(phenyl)methyl)-2-fluorocyclopropane-1-carboxamide;
(1R,2S)-2-fluoro-N—((S)-(6-fluoro-5-isopropylpyridin-2-yl)(1H-indazol-6-yl)methyl)cyclopropane-1-carboxamide;
(1R,2S)—N—((S)-(3-(1H-pyrazol-5-yl)phenyl)(6-fluoro-5-isopropylpyridin-2-yl)methyl)-2-fluorocyclopropane-1-carboxamide;
(1R,2S)-2-fluoro-N—((R)-(3-fluoro-4-isopropylphenyl)(2-oxo-2,3-dihydro-1H-benzo[d]imidazol-4-yl)methyl)cyclopropane-1-carboxamide;
(R)—N-((3-acetamidophenyl)(4-isopropylphenyl)methyl)cyclopropanecarboxamide;
(1R,2S)-2-cyano-N—((R)-(4-isopropylphenyl)(o-tolyl)methyl)cyclopentane-1-carboxamide;
N-((1R)-(4-isopropylphenyl)(2-((1-methyl-2,5-dioxoimidazolidin-4-yl)amino)phenyl)methyl)cyclopropanecarboxamide;
N-(2-((R)-((1R,2S)-2-fluorocyclopropane-1-carboxamido)(4-isopropylphenyl)methyl)phenyl)-1-(2,2,2-trifluoroethyl)piperidine-4-carboxamide;
(1R,2S)-2-fluoro-N—((R)-(3-fluoro-4-isopropylphenyl)(2-ureidophenyl)methyl)cyclopropane-1-carboxamide;
(1R,2S)-2-fluoro-N—((R)-(3-fluoro-4-isopropylphenyl)(1H-pyrazol-5-yl)methyl)cyclopropane-1-carboxamide;
N-(2-((R)-((1R,2S)-2-fluorocyclopropane-1-carboxamido)(4-isopropylphenyl)methyl)phenyl)-1-methylpiperidine-4-carboxamide;
N-(2-((R)-((1R,2S)-2-fluorocyclopropane-1-carboxamido)(4-isopropylphenyl)methyl)phenyl)-1-(2-methoxyethyl)piperidine-4-carboxamide;
1-(cyclopropylmethyl)-N-(2-((R)-((1R,2S)-2-fluorocyclopropane-1-carboxamido)(4-isopropylphenyl)methyl)phenyl)piperidine-4-carboxamide;
(R)—N-((4-isopropylphenyl)(2-oxo-2,3-dihydrobenzo[d]oxazol-7-yl)methyl)cyclopropanecarboxamide;
(1R,2S)—N1-((R)-(4-isopropylphenyl)(o-tolyl)methyl)-N2-methylcyclopentane-1,2-dicarboxamide;
(R)—N-((4-cyclopropylphenyl)(2-ureidophenyl)methyl)cyclopropanecarboxamide;
(R)—N-((4-cyclobutylphenyl)(2-ureidophenyl)methyl)cyclopropanecarboxamide;
(R,E)-N-((2-(2-cyanoguanidino)phenyl)(4-isopropylphenyl)methyl)cyclopropanecarboxamide;
methyl 3-((1S,2R)-2-(((R)-(4-isopropylphenyl)(o-tolyl)methyl)carbamoyl)cyclopentane-1-carboxamido)propanoate;
(1R,2S)-2-(2-acetamidoacetamido)-N—((S)-(4-isopropylphenyl)(phenyl)methyl)cyclopentane-1-carboxamide;
(1S,2R)—N1-cyano-N2-((R)-(4-isopropylphenyl)(o-tolyl)methyl)cyclopentane-1,2-dicarboxamide;
(R)—N-(2-(cyclopropanecarboxamido(4-isopropylphenyl)methyl)phenyl)-1-(2,2,2-trifluoroethyl)azetidine-3-carboxamide;
(R)—N-(2-(cyclopropanecarboxamido(4-isopropylphenyl)methyl)phenyl)-1-(2-methoxyethyl)azetidine-3-carboxamide;
(R)—N-(2-(cyclopropanecarboxamido(4-isopropylphenyl)methyl)phenyl)-1-(cyclopropylmethyl)azetidine-3-carboxamide;
(R)—N-(2-(cyclopropanecarboxamido(4-isopropylphenyl)methyl)phenyl)-1-methylazetidine-3-carboxamide;
(1S,2R)-2-fluoro-N—((S)-(4-isopropylphenyl)(phenyl)methyl)cyclopropane-1-carboxamide;
(S)-2-acetamido-N1-((1S,2R)-2-(((S)-(4-isopropylphenyl)(phenyl)methyl)carbamoyl)cyclopentyl)succinamide;
(R)—N-((4-isopropylphenyl)(2-oxo-2,3-dihydro-1H-benzo[d]imidazol-4-yl)methyl)cyclopropanecarboxamide;
(1R,2S)—N1-((R)-(4-isopropylphenyl)(o-tolyl)methyl)cyclopentane-1,2-dicarboxamide;
(R)—N-((4-isopropylphenyl)(2-(oxazol-2-ylamino)phenyl)methyl)cyclopropanecarboxamide;
(R)—N-((4-isopropylphenyl)(2-oxoindolin-7-yl)methyl)cyclopropanecarboxamide;
(S)—N-((5-isopropylpyridin-2-yl)(2-methoxyphenyl)methyl)cyclopropanecarboxamide;
(1S)-3-fluoro-N—((S)-(4-isopropylphenyl)(phenyl)methyl)cyclopentane-1-carboxamide;
(1S)-3-hydroxy-N—((S)-(4-isopropylphenyl)(phenyl)methyl)cyclopentane-1-carboxamide;
(1R,2S)-2-fluoro-N—((S)-(4-isopropylphenyl)(phenyl)methyl)cyclopropane-1-carboxamide;
(R)-1-acetyl-N-(2-(cyclopropanecarboxamido(4-isopropylphenyl)methyl)phenyl)azetidine-3-carboxamide;
(1R,2S)—N—((S)-(4-isopropylphenyl)(phenyl)methyl)-2-ureidocyclopentane-1-carboxamide;
(R)—N-((4-isopropylphenyl)(2-(methylamino)phenyl)methyl)cyclopropanecarboxamide;
(S)—N-((4-cyclobutylphenyl)(phenyl)methyl)cyclopropanecarboxamide;
(S)—N-((4-cyclopropylphenyl)(phenyl)methyl)cyclopropanecarboxamide;
(S)-1-acetyl-N-(2-((R)-cyclopropanecarboxamido(4-isopropylphenyl)methyl)phenyl)azetidine-2-carboxamide;
(R)—N-((4-isopropylphenyl)(3-methyl-1H-pyrazol-4-yl)methyl)cyclopropanecarboxamide;
(R)—N-((2-acetamido-5-fluorophenyl)(4-isopropylphenyl)methyl)cyclopropanecarboxamide;
(S)—N-((5-isopropylpyridin-2-yl)(phenyl)methyl)cyclopropanecarboxamide;
(S)—N-((2-acetamidophenyl)(5-isopropylpyridin-2-yl)methyl)cyclopropanecarboxamide;
(S)—N-((5-isopropylpyridin-2-yl)(o-tolyl)methyl)cyclopropanecarboxamide;
(R)—N-(2-(cyclopropanecarboxamido(4-isopropylphenyl)methyl)phenyl)azetidine-3-carboxamide;
(S)—N-(2-((R)-cyclopropanecarboxamido(4-isopropylphenyl)methyl)phenyl)azetidine-2-carboxamide;
(R)—N-(2-((R)-cyclopropanecarboxamido(4-isopropylphenyl)methyl)phenyl)azetidine-2-carboxamide;
(R)—N-((2-aminopyridin-3-yl)(4-isopropylphenyl)methyl)cyclopropanecarboxamide;
(S)—N-((3-fluoro-4-isopropylphenyl)(phenyl)methyl)cyclopropanecarboxamide;
(R)—N-((4-isopropylphenyl)(2-(3-methylureido)phenyl)methyl)cyclopropanecarboxamide;
(R)—N-((4-isopropylphenyl)(2-ureidophenyl)methyl)cyclopropanecarboxamide;
(1R,2S)-2-acetamido-N—((S)-(4-isopropylphenyl)(phenyl)methyl)cyclopentane-1-carboxamide;
(R)—N-((4-isopropylphenyl)(1H-pyrazol-5-yl)methyl)cyclopropanecarboxamide;
(R)—N-((4-isopropylphenyl)(2-(methylsulfonamido)phenyl)methyl)cyclopropanecarboxamide;
(R)—N-((2-chlorophenyl)(4-isopropylphenyl)methyl)cyclopropanecarboxamide;
(R)—N-((4-isopropylphenyl)(2-propionamidophenyl)methyl)cyclopropanecarboxamide;
(R)—N-(2-(cyclopropanecarboxamido(4-isopropylphenyl)methyl)phenyl)oxetane-3-carboxamide;
(R)—N-(2-(cyclopropanecarboxamido(4-isopropylphenyl)methyl)phenyl)cyclopropanecarboxamide;
(R)—N-((3-hydroxyphenyl)(4-isopropylphenyl)methyl)cyclopropanecarboxamide;
(R)—N-((4-isopropylphenyl)(3-methoxy-2-methylphenyl)methyl)cyclopropanecarboxamide;
(R)—N-((2-aminophenyl)(4-isopropylphenyl)methyl)cyclopropanecarboxamide;
(R)—N-((4-isopropylphenyl)(o-tolyl)methyl)cyclopropanecarboxamide;
(R)—N-((4-isopropylphenyl)(m-tolyl)methyl)cyclopropanecarboxamide;
(R)—N-((2-hydroxyphenyl)(4-isopropylphenyl)methyl)cyclopropanecarboxamide;
(R)—N-((4-hydroxyphenyl)(4-isopropylphenyl)methyl)cyclopropanecarboxamide;
(R)—N-((4-isopropylphenyl)(1-methyl-1H-pyrazol-5-yl)methyl)cyclopropanecarboxamide;
(R)—N-((4-isopropylphenyl)(3-methoxyphenyl)methyl)cyclopropanecarboxamide;
(R)—N-((4-isopropylphenyl)(2-methoxyphenyl)methyl)cyclopropanecarboxamide;
(R)—N-((4-isopropylphenyl)(4-methoxyphenyl)methyl)cyclopropanecarboxamide;
(S)—N-((4-isopropylphenyl)(phenyl)methyl)cyclopropanecarboxamide; and
   or a stereoisomer or tautomer thereof, or a pharmaceutically acceptable salt of any of the foregoing.

### Methods of Treatment

Provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I), or any variation or embodiment thereof, or a stereoisomer or tautomer thereof, is selective for GYS1 over GYS2. In some embodiments, the compound of formula (I), or any variation or embodiment thereof, or a stereoisomer or tautomer thereof, is greater than 500 or 1,000 or 1,500 or 1,700-fold selective for GYS1 over GYS2.

Provided herein is a method of inhibiting GYS1 in a cell, comprising exposing the cell to (i) a composition comprising an effective amount of a GYS1 inhibitor, or (ii) a pharmaceutical composition, comprising an effective amount of a GYS1 inhibitor, and one or more pharmaceutically acceptable excipients. In some embodiments, the GYS1 inhibitor is a small molecule. In some embodiments, the GYS1 inhibitor is selective for GYS1 over GYS2. In some embodiments, the GYS1 inhibitor is greater than 500 or 1,000 or 1,500 or 1,700-fold selective for GYS1 over GYS2.

Provided herein is a method of inhibiting GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

In some embodiments, the compound of formula (I), or any variation or embodiment thereof, or a stereoisomer or tautomer thereof, or a pharmaceutically acceptable salt of any of the foregoing, is selective for GYS1 over GYS2. In some embodiments, the compound of formula (I), or any variation or embodiment thereof, or a stereoisomer or tautomer thereof, or a pharmaceutically acceptable salt of any of the foregoing, is greater than 500 or 1,000 or 1,500 or 1,700-fold selective for GYS1 over GYS2. In some embodiments, the individual has a GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML). In some embodiments, the GYS1-mediated disease, disorder, or condition is Pompe disease. In some embodiments, the GYS1-mediated disease, disorder, or condition is late-onset Pompe disease (LOPD).

Provided herein is a method of reducing tissue glycogen stores in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of inhibiting glycogen synthesis in an individual in need thereof, comprising administering to the individual an effective amount of (i) compound of formula (I) or any variation or embodiment thereof, or a stereoisomer or tautomer thereof, or a pharmaceutically acceptable salt of any of the foregoing, or (ii) a pharmaceutical composition, comprising compound of formula (I) or any variation or embodiment thereof, or a stereoisomer or tautomer thereof, or a pharmaceutically acceptable salt of any of the foregoing, and one or more pharmaceutically acceptable excipients.

Provided herein is a method of treating a GYS1-mediated disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

Provided herein is a method of treating a glycogen storage disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating a glycogen storage disease, disorder, or condition in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating Pompe disease in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

Provided herein is a method of treating Pompe disease in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

In some embodiments, the compounds provided herein reduce and/or eliminate one or more symptoms associated with Pompe disease. In some embodiments, the compounds reduce and/or eliminate weak muscles, poor muscle tone, enlarged liver, failure to grow and gain weight, trouble breathing, feeding problems, infections in the respiratory system, problems with hearing, motor skill delay, heart enlargement, tiredness, lung infection, frequent falling, or irregular heartbeat. In some embodiments, the compounds herein delay progression of Pompe disease.

In some embodiments, the compounds provided herein increase the lifespan of the individual. In some embodiments, the lifespan is increased at least 5, at least 10, or at least 20 years upon treatment.

In some embodiments, the compounds provided herein prevent, reduce, or delay muscle weakness. In some embodiments, muscle weakness is determined by manual muscle testing, sit to stand test, heel-raise test, hand-held dynamometry, or hand grip dynamometry. In some embodiments, strength is graded according to the following scale: 0: No visible muscle contraction; 1: Visible muscle contraction with no or trace movement; 2: Limb movement, but not against gravity; 3: Movement against gravity but not resistance; 4: Movement against at least some resistance supplied by the examiner; 5: Full strength.

Also provided herein is a method of inhibiting a GYS1 enzyme in an individual comprising administering an effective amount of a compound of formula (I) or a pharmaceutically acceptable salt thereof to the individual. In some embodiments the GYS1 enzyme is human GYS1 (hGYS1). In some embodiments, the compounds provided herein are inhibit GYS1 at a concentration of less than 10 μM, less than 1 μM, less than 0.5 μM, or less than 0.1 μM. In some embodiments, the compounds provided herein inhibit GYS1 at a concentration of 1-10 μM, 0.01 to 1 μM, or 0.01 to 10 μM.

In some embodiments, the compounds have an IC₅₀ of less than 10 nM, less than 10 μM, less than 1 μM, less than 0.5 μM, or less than 0.1 μM. In some embodiments, the compounds provided herein have an IC₅₀ of 1 to 10 nM, 1 to 10 μM, 0.01 to 1 μM, 0.01 to 10 μM, or 0.001 to 0.01 μM.

In some embodiments, glycogen synthesis is inhibited upon administration of a compound provided herein. In some embodiments, glycogen synthesis is reduced at least 10%, at least 20%, at least 40% or at least 50% upon administration.

In some embodiments, the individual receiving treatment is a juvenile human or an infant. In some embodiments, the individual is less than 10 years old, less than 9 years old, less than 8 years old, less than 7 years old, less than 6 years old, less than 5 years old, less than 4 years old, less than 3 years old, less than 2 years old, or less than one year old.

In some embodiments, the methods further comprise enzyme replacement therapy (ERT). Exemplary ERTs include alglucosidase alfa (human recombinant alpha-glucosidase (human GAA)) and those described in Byrne B J et al (2011). Pompe disease: design, methodology, and early findings from the Pompe Registry. Mol Genet Metab 103: 1-11 (herein incorporated by reference in its entirety). In some embodiments, the ERT is selected from the group consisting of Myozyme and Lumizyme. In some embodiments, the ERT is Myozyme. In some embodiments, the ERT is Lumizyme. In some embodiments, the individual has an advanced glycogen storage disease. In some embodiments, the individual has late onset Pompe Disease. Thus, provided herein is a method of treating a GYS1-mediated disease, disorder, or condition in an individual in need thereof, comprising subjecting the individual to (a) glycogen substrate reduction therapy, such as administering to the individual an effective amount of (i) compound of formula (I), or any variation or embodiment thereof, or a stereoisomer or tautomer thereof, or a pharmaceutically acceptable salt of any of the foregoing, or (ii) a pharmaceutical composition comprising compound of formula (I), or any variation or embodiment thereof, or a stereoisomer or tautomer thereof, or a pharmaceutically acceptable salt of any of the foregoing, and one or more pharmaceutically acceptable excipients, and (b) enzyme replacement therapy. In some embodiments, the GYS1-mediated disease, disorder, or condition is Pompe disease, such as late-onset Pompe disease. In some embodiments, the compound of formula (I) is selective for GYS1 over GYS2. In some embodiments, the compound of formula (I) is greater than 500 or 1,000 or 1,500 or 1,700-fold selective for GYS1 over GYS2.

In some embodiments, the individual has a mutation in the GAA gene. In some embodiments, the mutation reduces the level of GAA protein. In some embodiments, the mutation is a loss-of-function mutation. In some embodiments, the mutation is a missense mutation. In some embodiments, the mutation is a deletion. In some embodiments, the mutation is a recessive mutation. In some embodiments, the mutation is a splicing variant.

In some embodiments of the foregoing, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a composition comprising an effective amount of a compound of formula (I), or any variation or embodiment thereof, or a stereoisomer or tautomer thereof, or a pharmaceutically acceptable salt of any of the foregoing, and (ii) instructions for use in treating an GYS1-mediated disease, disorder, or condition in an individual in need thereof. Also provided herein are kits, comprising (i) a pharmaceutical composition comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients; and (ii) instructions for use in treating an GYS1-mediated disease, disorder, or condition in an individual in need thereof

Articles of manufacture are also provided, wherein the article of manufacture comprises a compound of formula (I), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (I), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Preparing

The present disclosure further provides processes for preparing the compounds of present invention. In some aspects, provided herein are processes of preparing a compound of (I), (I-A), (I-A1), (I-A2), (I-B), (I-B1), (I-B2), (I-B3), (I-B4), (I-B5), (I-B6), (I-C), (I-D), (I-E), (I-E1), (I-E2), (I-F), (I-F1), (I-G), (1-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing.

In some embodiments, a process for preparing a compound of formula (I), or a stereoisomer or tautomer thereof, or a pharmaceutically acceptable salt of any of the foregoing, comprises
(a) reacting a compound of formula (I-1):

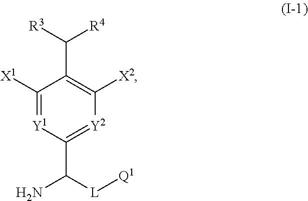

or a salt thereof, wherein
Y¹ and Y² are each CH, or
one of Y¹ and Y² is N and the other of Y¹ and Y² is CH;
X¹ and X² are each independently H or halo;
R³ and R⁴ are each —CH₃, or
R³ and R⁴ are taken, together with the atoms to which they are attached, to form cyclopropyl or cyclobutyl;
either
(1) L is absent; and

### Q¹ is:

(i) C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl, or
   (ii) 3-15 membered heterocyclyl, wherein the 3-15 membered heterocyclyl of Q¹ is optionally substituted with one or more oxo, or
   (iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl;
   or

### (2) L is —CH₂—; and

Q¹ is C₃₋₁₀cycloalkyl,
with a compound of formula (I-2):

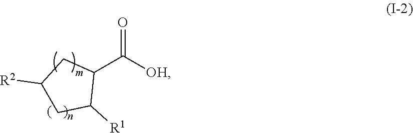

wherein,
m is 0 or 1;
n is 0 or 1;
R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein
the C₁₋₆alkyl of the —C(O)—NH(C₁₋₆alkyl) of R¹ is optionally substituted with one or more —C(O)—C₁₋₆alkoxy, and
the C₁₋₆alkyl of the —NH—C(O)—C₁₋₆alkyl of R¹ is optionally substituted with one or more —NH—C(O)—C₁₋₆alkyl or —C(O)—NH₂; and
   R² is H, halo, or —OH
   in the presence of a coupling reagent to provide a compound of formula (I).

In some embodiments, the coupling reagent comprises propanephosphonic acid anhydride (T3P), or N,N,N′,N′-tetramethylchloroformamidinium hexafluorophosphate (TFCH).

In some embodiments, the process further comprises the presence of a base. In some embodiments, the base comprises an amine. In some embodiments, the base comprises a tertiary amine. In some embodiments, the amine is N-methylmorpholine or N-methylimidazole.

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### Synthetic Examples

As depicted in the Schemes and Examples below, in certain exemplary embodiments, compounds of formula (I), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

### Schemes

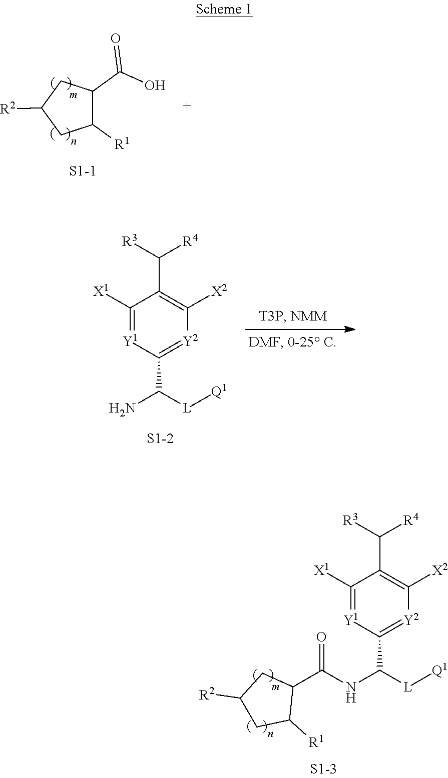

Compounds of formula S1-3 can be prepared according to general Scheme 1. Reaction of carboxylic acid S1-1 with amine S1-2 using a coupling reagent such as propanephosphonic acid anhydride (T3P) and a tertiary amine base such as N-methylmorpholine in an aprotic solvent such as DMF provides compounds of formula S1-3.

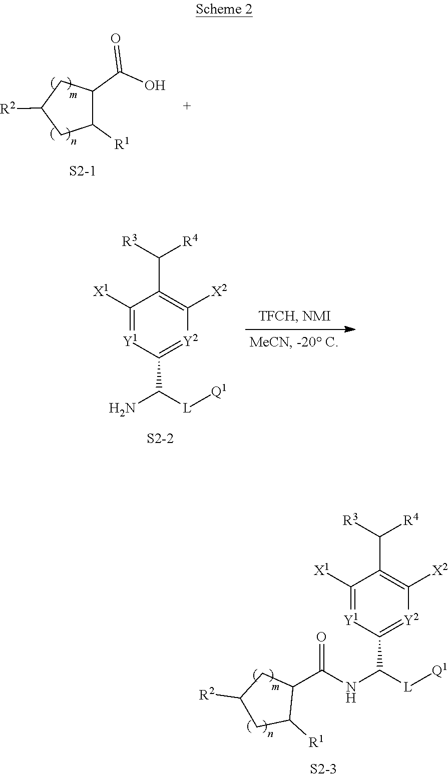

Compounds of formula S2-3 can be prepared according to the alternative reaction conditions shown in general Scheme 2. Reaction of carboxylic acid S2-1 with amine S2-2 using a coupling reagent such as N,N,N′,N′-tetramethylchloroformamidinium hexafluorophosphate (TFCH) and a tertiary amine base such as N-methylimidazole in an aprotic solvent such as acetonitrile provides compounds of formula S2-3.

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | | | |
|---|---|---|---|
| ° C. | degrees Celsius | K₂CO₃ | potassium carbonate |
| μL | microliter | LDA | lithium diisopropylamide |
| [M + XX]⁺ | observed mass | LiHMDS | lithium |
| AC₅₀ | half-maximal activity | | bis(trimethylsilyl)amide |
| | concentration | MeOH | Methanol |
| ACN | acetonitrile | MeCN | acetonitrile |
| app | apparent (NMR) | m | multiplet (NMR) |
| BH₃•THF | borane-tetrahydrofuran | mg | milligrams |
| | complex | min | minutes |
| BBr₃ | boron tribromide | mL | milliliter |
| Calc'd | calculated | mmol | millimole |
| Cbz-Cl | benzyl chloroformate | mM | millimolar |
| CO₂ | carbon dioxide | M | molarity or molar |
| Cs₂CO₃ | cesium carbonate | MS | mass spectrometry |
| d | deuterated (NMR | MsCl | methanesulfonyl chloride |
| | solvents) | MTBE | methyl tert-butyl ether |
| d | doublet (NMR) | n/a | not applicable |
| dd | doublet of doublets | NBS | N-bromosuccinimide |
| | (NMR) | NH₄ | ammonium |
| DCM | dichloromethane | NH₄OH | ammonium hydroxide |
| DIAD | diisopropyl | NH₄HCO₃ | ammonium bicarbonate |
| | azodicarboxylate | Na₂SO₄ | sodium sulfate |
| DMF | N,N-dimethylformamide | NaBH₃CN | sodium |
| EC₅₀ | half-maximal effective | | cyanoborohydride |
| | concentration | NMI | N-methylimidazole |
| EDCI | 1-Ethyl-3-(3- | NMM | N-methylmorpholine |
| | dimethylaminopropyl)carbodiimide | NMR | nuclear magnetic |
| | | | resonance |
| ESI | electrospray ionization | NaOH | sodium hydroxide |
| EtOAc | ethyl acetate | PCy₃ | Tricyclohexylphosphine |
| EtOH | ethanol | PdCl₂(dppf) | [1,1′- |
| eq | equivalents | | Bis(diphenylphosphino)fer- |
| g | grams | | rocene]dichloropalladium(II) |
| h | hours | | |
| H | hydrogen | pH | potential of hydrogen |
| HCl | hydrochloric acid | PPh₃ | triphenyl phosphine |
| HPLC | high-performance liquid | s | singlet (NMR) |
| | chromatography | SFC | super fluid |
| IC₅₀ | half-maximal inhibitory | | chromatography |
| | concentration | t | triplet (NMR) |
| In vacuo | in a vacuum | T3P | Propanephosphonic acid |
| IUPAC | International Union of | | anhydride |
| | Pure and Applied | TBAB | tetrabutylammonium |
| | Chemistry | | bromide |
| MHz | megahertz | TEA | triethylamine |
| J | J-coupling value (NMR) | TFA | trifluoroacetic acid |
| TFCH | N,N,N′,N′- | THF | tetrahydrofuran |
| | tetramethylchloroformamidinium | TMSCl | trimethylsilyl chloride |
| | hexafluorophosphate | wt. % | weight percent |

### Example S-1

TABLE 2

| | | Exact | Observed |
|---|---|---|---|
| Cmpd | | Mass | Mass |
| No. | Cmpd Name | (g/mol) | (M + H) |
| 1 | (R)-N-((2-acetamidophenyl)(4- | 350.2 | 351.2 |
| | isopropylphenyl)methyl)cyclopropanecarboxamide | | |
| 2 | (1R,2S)-N-((S)-(5-cyclopropyl-6-fluoropyridin-2- | 328.1 | 329.2 |
| | yl)(phenyl)methyl)-2-fluorocyclopropane-1-carboxamide | | |
| 3 | (1R,2S)-2-fluoro-N-((S)-(6-fluoro-5-isopropylpyridin-2- | 370.2 | 371.1 |
| | yl)(1H-indazol-6-yl)methyl)cyclopropane-1-carboxamide | | |
| 4 | (1R,2S)-N-((S)-(3-(1H-pyrazol-5-yl)phenyl)(6-fluoro-5- | 396.2 | 397.1 |
| | isopropylpyridin-2-yl)methyl)-2-fluorocyclopropane-1- | | |
| | carboxamide | | |
| 5 | (1R,2S)-2-fluoro-N-((R)-(3-fluoro-4-isopropylphenyl)(2-oxo- | 385.2 | 386.0 |
| | 2,3-dihydro-1H-benzo[d]imidazol-4- | | |
| | yl)methyl)cyclopropane-1-carboxamide | | |
| 6 | (R)-N-((3-acetamidophenyl)(4- | 350.2 | 351.2 |
| | isopropylphenyl)methyl)cyclopropanecarboxamide | | |
| 7 | (1R,2S)-2-cyano-N-((R)-(4-isopropylphenyl)(o- | 360.2 | 361.3 |
| | tolyl)methyl)cyclopentane-1-carboxamide | | |
| 8 | N-((1R)-(4-isopropylphenyl)(2-((1-methyl-2,5- | 420.2 | 419.1 (M − H) |
| | dioxoimidazolidin-4- | | |
| | yl)amino)phenyl)methyl)cyclopropanecarboxamide | | |
| 9 | N-(2-((R)-((1R,2S)-2-fluorocyclopropane-1-carboxamido)(4- | 519.3 | 520.3 |
| | isopropylphenyl)methyl)phenyl)-1-(2,2,2- | | |
| | trifluoroethyl)piperidine-4-carboxamide | | |
| 10 | (1R,2S)-2-fluoro-N-((R)-(3-fluoro-4-isopropylphenyl)(2- | 387.2 | 388.1 |
| | ureidophenyl)methyl)cyclopropane-1-carboxamide | | |
| 11 | (1R,2S)-2-fluoro-N-((R)-(3-fluoro-4-isopropylphenyl)(1H- | 319.1 | 320.1 |
| | pyrazol-5-yl)methyl)cyclopropane-1-carboxamide | | |
| 12 | N-(2-((R)-((1R,2S)-2-fluorocyclopropane-1-carboxamido)(4- | 451.3 | 452.2 |
| | isopropylphenyl)methyl)phenyl)-1-methylpiperidine-4- | | |
| | carboxamide | | |
| 13 | N-(2-((R)-((1R,2S)-2-fluorocyclopropane-1-carboxamido)(4- | 495.3 | 496.3 |
| | isopropylphenyl)methyl)phenyl)-1-(2- | | |
| | methoxyethyl)piperidine-4-carboxamide | | |
| 14 | 1-(cyclopropylmethyl)-N-(2-((R)-((1R,2S)-2- | 491.3 | 492.3 |
| | fluorocyclopropane-1-carboxamido)(4- | | |
| | isopropylphenyl)methyl)phenyl)piperidine-4-carboxamide | | |
| 15 | (R)-N-((4-isopropylphenyl)(2-oxo-2,3- | 350.2 | 351.1 |
| | dihydrobenzo[d]oxazol-7- | | |
| | yl)methyl)cyclopropanecarboxamide | | |
| 16 | (1R,2S)-N¹-((R)-(4-isopropylphenyl)(o-tolyl)methyl)-N²- | 392.2 | 393.2 |
| | methylcyclopentane-1,2-dicarboxamide | | |
| 17 | (R)-N-((4-cyclopropylphenyl)(2- | 349.2 | 350.2 |
| | ureidophenyl)methyl)cyclopropanecarboxamide | | |
| 18 | (R)-N-((4-cyclobutylphenyl)(2- | 363.2 | 364.1 |
| | ureidophenyl)methyl)cyclopropanecarboxamide | | |
| 19 | (R,E)-N-((2-(2-cyanoguanidino)phenyl)(4- | 375.2 | 376.2 |
| | isopropylphenyl)methyl)cyclopropanecarboxamide | | |
| 20 | methyl 3-((1S,2R)-2-(((R)-(4-isopropylphenyl)(o- | 464.3 | 465.2 |
| | tolyl)methyl)carbamoyl)cyclopentane-1- | | |
| | carboxamido)propanoate | | |
| 21 | (1R,2S)-2-(2-acetamidoacetamido)-N-((S)-(4- | 435.3 | 436.3 |
| | isopropylphenyl)(phenyl)methyl)cyclopentane-1- | | |
| | carboxamide | | |
| 22 | (1S,2R)-N¹-cyano-N²-((R)-(4-isopropylphenyl)(o- | 403.2 | 404.3 |
| | tolyl)methyl)cyclopentane-1,2-dicarboxamide | | |
| 23 | (R)-N-(2-(cyclopropanecarboxamido(4- | 473.2 | 474.1 |
| | isopropylphenyl)methyl)phenyl)-1-(2,2,2- | | |
| | trifluoroethyl)azetidine-3-carboxamide | | |
| 24 | (R)-N-(2-(cyclopropanecarboxamido(4- | 449.3 | 450.1 |
| | isopropylphenyl)methyl)phenyl)-1-(2- | | |
| | methoxyethyl)azetidine-3-carboxamide | | |
| 25 | (R)-N-(2-(cyclopropanecarboxamido(4- | 445.3 | 446.3 |
| | isopropylphenyl)methyl)phenyl)-1- | | |
| | (cyclopropylmethyl)azetidine-3-carboxamide | | |
| 26 | (R)-N-(2-(cyclopropanecarboxamido(4- | 405.2 | 406.1 |
| | isopropylphenyl)methyl)phenyl)-1-methylazetidine-3- | | |
| | carboxamide | | |
| 27 | (1S,2R)-2-fluoro-N-((S)-(4- | 311.2 | 312.1 |
| | isopropylphenyl)(phenyl)methyl)cyclopropane-1- | | |
| | carboxamide | | |
| 28 | (S)-2-acetamido-N¹-((1S,2R)-2-(((S)-(4- | 492.3 | 493.2 |
| | isopropylphenyl)(phenyl)methyl)carbamoyl)cyclopentyl)succinamide | | |
| 29 | (R)-N-((4-isopropylphenyl)(2-oxo-2,3-dihydro-1H- | 349.2 | 350.1 |
| | benzo[d]imidazol-4-yl)methyl)cyclopropanecarboxamide | | |
| 30 | (1R,2S)-N¹-((R)-(4-isopropylphenyl)(o- | 378.2 | 379.2 |
| | tolyl)methyl)cyclopentane-1,2-dicarboxamide | | |
| 31 | (R)-N-((4-isopropylphenyl)(2-(oxazol-2- | 375.2 | 376.2 |
| | ylamino)phenyl)methyl)cyclopropanecarboxamide | | |
| 32 | (R)-N-((4-isopropylphenyl)(2-oxoindolin-7- | 348.2 | 349.2 |
| | yl)methyl)cyclopropanecarboxamide | | |
| 33 | (S)-N-((5-isopropylpyridin-2-yl)(2- | 324.2 | 325.2 |
| | methoxyphenyl)methyl)cyclopropanecarboxamide | | |
| 34 | (1S)-3-fluoro-N-((S)-(4- | 339.2 | 340.1 |
| | isopropylphenyl)(phenyl)methyl)cyclopentane-1- | | |
| | carboxamide | | |
| 35 | (1S)-3-hydroxy-N-((S)-(4- | 337.2 | 338.1 |
| | isopropylphenyl)(phenyl)methyl)cyclopentane-1- | | |
| | carboxamide | | |
| 36 | (1R,2S)-2-fluoro-N-((S)-(4- | 311.2 | 312.1 |
| | isopropylphenyl)(phenyl)methyl)cyclopropane-1- | | |
| | carboxamide | | |
| 37 | (R)-1-acetyl-N-(2-(cyclopropanecarboxamido(4- | 433.2 | 434.2 |
| | isopropylphenyl)methyl)phenyl)azetidine-3-carboxamide | | |
| 38 | (1R,2S)-N-((S)-(4-isopropylphenyl)(phenyl)methyl)-2- | 379.2 | 380.3 |
| | ureidocyclopentane-1-carboxamide | | |
| 39 | (R)-N-((4-isopropylphenyl)(2- | 322.2 | 323.2 |
| | (methylamino)phenyl)methyl)cyclopropanecarboxamide | | |
| 40 | (S)-N-((4- | 305.2 | 306.1 |
| | cyclobutylphenyl)(phenyl)methyl)cyclopropanecarboxamide | | |
| 41 | (S)-N-((4- | 291.2 | 292.1 |
| | cyclopropylphenyl)(phenyl)methyl)cyclopropanecarboxamide | | |
| 42 | (S)-1-acetyl-N-(2-((R)-cyclopropanecarboxamido(4- | 433.2 | 434.3 |
| | isopropylphenyl)methyl)phenyl)azetidine-2-carboxamide | | |
| 43 | (R)-N-((4-isopropylphenyl)(3-methyl-1H-pyrazol-4- | 297.2 | 298.2 |
| | yl)methyl)cyclopropanecarboxamide | | |
| 44 | (R)-N-((2-acetamido-5-fluorophenyl)(4- | 368.2 | 369.1 |
| | isopropylphenyl)methyl)cyclopropanecarboxamide | | |
| 45 | (S)-N-((5-isopropylpyridin-2- | 294.2 | 294.9 |
| | yl)(phenyl)methyl)cyclopropanecarboxamide | | |
| 46 | (S)-N-((2-acetamidophenyl)(5-isopropylpyridin-2- | 351.2 | 352.1 |
| | yl)methyl)cyclopropanecarboxamide | | |
| 47 | (S)-N-((5-isopropylpyridin-2-yl)(o- | 308.2 | 309.2 |
| | tolyl)methyl)cyclopropanecarboxamide | | |
| 48 | (R)-N-(2-(cyclopropanecarboxamido(4- | 391.2 | 392.4 |
| | isopropylphenyl)methyl)phenyl)azetidine-3-carboxamide | | |
| 49 | (S)-N-(2-((R)-cyclopropanecarboxamido(4- | 391.2 | 392.4 |
| | isopropylphenyl)methyl)phenyl)azetidine-2-carboxamide | | |
| 50 | (R)-N-(2-((R)-cyclopropanecarboxamido(4- | 391.2 | 392.4 |
| | isopropylphenyl)methyl)phenyl)azetidine-2-carboxamide | | |
| 51 | (R)-N-((2-aminopyridin-3-yl)(4- | 309.2 | 310.2 |
| | isopropylphenyl)methyl)cyclopropanecarboxamide | | |
| 52 | (S)-N-((3-fluoro-4- | 311.2 | 312.2 |
| | isopropylphenyl)(phenyl)methyl)cyclopropanecarboxamide | | |
| 53 | (R)-N-((4-isopropylphenyl)(2-(3- | 365.2 | 366.2 |
| | methylureido)phenyl)methyl)cyclopropanecarboxamide | | |
| 54 | (R)-N-((4-isopropylphenyl)(2- | 351.2 | 352.2 |
| | ureidophenyl)methyl)cyclopropanecarboxamide | | |
| 55 | (1R,2S)-2-acetamido-N-((S)-(4- | 378.2 | 379.2 |
| | isopropylphenyl)(phenyl)methyl)cyclopentane-1- | | |
| | carboxamide | | |
| 56 | (R)-N-((4-isopropylphenyl)(1H-pyrazol-5- | 283.2 | 284.2 |
| | yl)methyl)cyclopropanecarboxamide | | |
| 57 | (R)-N-((4-isopropylphenyl)(2- | 386.2 | 387.2 |
| | (methylsulfonamido)phenyl)methyl)cyclopropanecarboxamide | | |
| 58 | (R)-N-((2-chlorophenyl)(4- | 327.1 | 328.1 |
| | isopropylphenyl)methyl)cyclopropanecarboxamide | | |
| 59 | (R)-N-((4-isopropylphenyl)(2- | 364.2 | 365.4 |
| | propionamidophenyl)methyl)cyclopropanecarboxamide | | |
| 60 | (R)-N-(2-(cyclopropanecarboxamido(4- | 392.2 | 393.3 |
| | isopropylphenyl)methyl)phenyl)oxetane-3-carboxamide | | |
| 61 | (R)-N-(2-(cyclopropanecarboxamido(4- | 376.2 | 377.2 |
| | isopropylphenyl)methyl)phenyl)cyclopropanecarboxamide | | |
| 62 | (R)-N-((3-hydroxyphenyl)(4- | 309.2 | 310.1 |
| | isopropylphenyl)methyl)cyclopropanecarboxamide | | |
| 63 | (R)-N-((4-isopropylphenyl)(3-methoxy-2- | 337.2 | 338.2 |
| | methylphenyl)methyl)cyclopropanecarboxamide | | |
| 64 | (R)-N-((2-aminophenyl)(4- | 308.2 | 309.3 |
| | isopropylphenyl)methyl)cyclopropanecarboxamide | | |
| 65 | (R)-N-((4-isopropylphenyl)(o- | 307.2 | 308.3 |
| | tolyl)methyl)cyclopropanecarboxamide | | |
| 66 | (R)-N-((4-isopropylphenyl)(m- | 307.2 | 308.2 |
| | tolyl)methyl)cyclopropanecarboxamide | | |
| 67 | (R)-N-((2-hydroxyphenyl)(4- | 309.2 | 310.1 |
| | isopropylphenyl)methyl)cyclopropanecarboxamide | | |
| 68 | (R)-N-((4-hydroxyphenyl)(4- | 309.2 | 310.1 |
| | isopropylphenyl)methyl)cyclopropanecarboxamide | | |
| 69 | (R)-N-((4-isopropylphenyl)(1-methyl-1H-pyrazol-5- | 297.2 | 298.2 |
| | yl)methyl)cyclopropanecarboxamide | | |
| 70 | (R)-N-((4-isopropylphenyl)(3- | 323.2 | 324.1 |
| | methoxyphenyl)methyl)cyclopropanecarboxamide | | |
| 71 | (R)-N-((4-isopropylphenyl)(2- | 323.2 | 324.2 |
| | methoxyphenyl)methyl)cyclopropanecarboxamide | | |
| 72 | (R)-N-((4-isopropylphenyl)(4- | 323.2 | 324.2 |
| | methoxyphenyl)methyl)cyclopropanecarboxamide | | |
| 73 | (S)-N-((4- | 293.2 | 294.1 |
| | isopropylphenyl)(phenyl)methyl)cyclopropanecarboxamide | | |

### Intermediate A-1: Synthesis of (6-fluoro-5-isopropylpyridin-2-yl)(1H-indazol-6-yl)methanaminium chloride

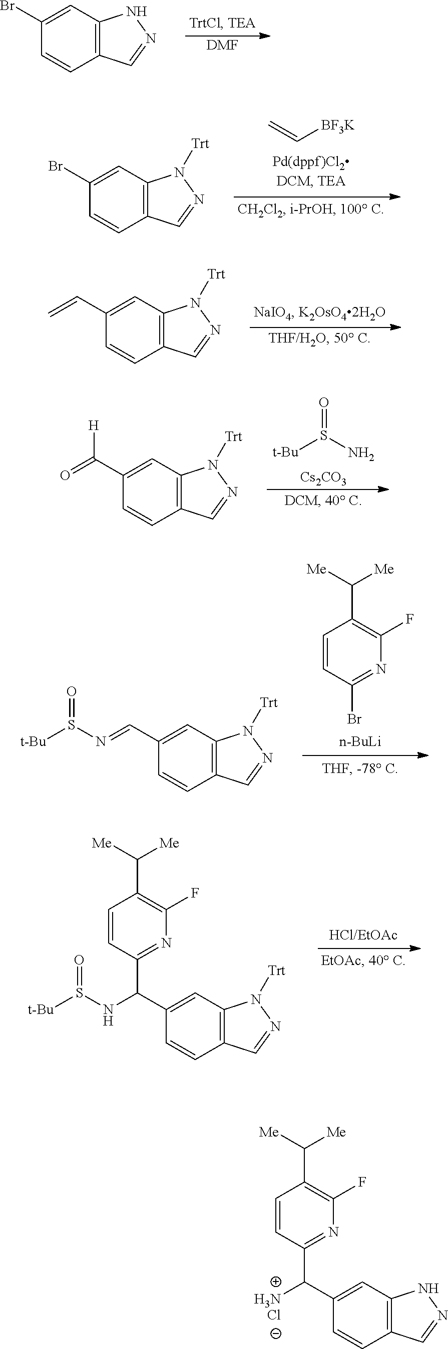

Step a: To a solution of 6-bromo-1H-indazole (8 g, 40.6 mmol, 1 eq) in DMF (50 mL) was added trityl chloride (TrtCl, 12.4 g, 44.6 mmol, 1.1 eq) and TEA (7.06 mL, 50.7 mmol, 1.25 eq). The resulting mixture was stirred at 25° C. for 16 h. The reaction mixture was then diluted with water, and the resulting biphasic mixture was extracted with ethyl acetate (3×50 mL). The combined organic extracts were dried over anhydrous Na₂SO₄, filtered, and concentrated under reduced pressure. The crude residue obtained was triturated with MTBE (30 mL) and filtered to give 6-bromo-1-trityl-1H-indazole, which was carried forward to the next step without further purification or characterization.

Step b: To a mixture of 6-bromo-1-trityl-1H-indazole (16.7 g, 38.0 mmol, 1 eq), potassium vinyltrifluoroborate (10.1 g, 76.0 mmol, 2 eq) and TEA (15.8 mL, 14.0 mmol, 3 eq) in i-PrOH (160 mL), was added Pd(dppf)Cl₂·CH₂Cl₂ (1.55 g, 1.90 mmol, 0.05 eq) under N2. The resulting mixture was then degassed and placed under an N2 atmosphere. The reaction mixture was then warmed to 100° C. and stirred for 2 h under N2. After cooling, the mixture was filtered, and the filter cake was washed with ethyl acetate (3×100 mL). The combined filtrates were concentrated, and the crude residue obtained was purified by column chromatography to give 1-trityl-6-vinyl-1H-indazole. LC-MS (ESI): m/z: [2M+Na]⁺ calculated for C₂₈H₂₂N₂: 795.4; found 795.3.

Step c: To a solution of 1-trityl-6-vinyl-1H-indazole (14.2 g, 36.7 mmol, 1 eq) in THF:H₂O (5:1) (300 mL) at 0° C. was added NaIO₄ (31.4 g, 146 mmol, 4 eq) and K₂OsO₄·2H₂O (676 mg, 1.84 mmol, 0.05 eq). The resulting mixture was warmed to 50° C. and stirred for 1 h. The reaction mixture was then cooled to 25° C. and quenched with sat. aq. Na₂S₂O₃ (100 mL). The resulting mixture was extracted with ethyl acetate (3×100 mL), and the combined extracts were dried over Na₂SO₄, filtered, and concentrated under reduced pressure. The resulting residue was purified by column chromatography to give 1-trityl-1H-indazole-6-carbaldehyde.

Step d: To a solution 1-trityl-1H-indazole-6-carbaldehyde (7.3 g, 18.8 mmol, 1 eq) in DCM (75 mL) was added Cs₂CO₃ (6.74 g, 20.7 mmol, 1.1 eq) and 2-methylpropane-2-sulfinamide (2.51 g, 20.6 mmol, 1.1 eq). The mixture was then warmed to 40° C. and stirred for 16 h. The reaction mixture was then filtered, and the filter cake was washed with ethyl acetate (3×100 mL). The filtrate was then filtered and concentrated under reduced pressure. The crude residue obtained was purified by column chromatography to give (E)-2-methyl-N-((1-trityl-1H-indazol-6-yl)methylene)propane-2-sulfinamide.

Step e: To a solution of 6-bromo-2-fluoro-3-isopropylpyridine (665 mg, 3.05 mmol, 1.5 eq) in THE (5 mL) at −78° C. under N2 was added n-BuLi (1.22 mL, 2.5 M, 1.5 eq). The resulting mixture was stirred at −78° C. for 0.5 h. After this time, (E)-2-methyl-N-((1-trityl-1H-indazol-6-yl)methylene)propane-2-sulfinamide (1 g, 2.03 mmol, 1 eq) in THE (5 mL) cooled to −78° C. under N2 was added, and the resulting mixture was stirred at −78° C. for 4 h. The reaction was then quenched with saturated aqueous NH₄Cl (20 mL), and the resulting biphasic mixture was extracted with ethyl acetate (3×20 mL), dried over anhydrous Na₂SO₄, filtered, and concentrated under reduced pressure. The crude residue obtained was purified by column chromatography to give N-((6-fluoro-5-isopropylpyridin-2-yl)(1-trityl-1H-indazol-6-yl)methyl)-2-methylpropane-2-sulfinamide.

Step f: To a solution of N-((6-fluoro-5-isopropylpyridin-2-yl)(1-trityl-1H-indazol-6-yl)methyl)-2-methylpropane-2-sulfinamide (600 mg, 951 μmol, 1 eq) in EtOAc (3 mL) at 0° C. was added HCl/EtOAc (4 M, 3 mL, 12.6 eq). The resulting mixture was then warmed to 40° C. and stirred for 16 h. The reaction mixture was then filtered and concentrated to give (6-fluoro-5-isopropylpyridin-2-yl)(1H-indazol-6-yl)methanaminium chloride. LC-MS (ESI): m/z: [M-NH₃]⁺ calculated for C₁₆H₁₇FN₄: 268.1; found 268.2.

### Intermediate A-2: Synthesis of (3-(1H-pyrazol-5-yl)phenyl)(6-fluoro-5-isopropylpyridin-2-yl)methanaminium chloride

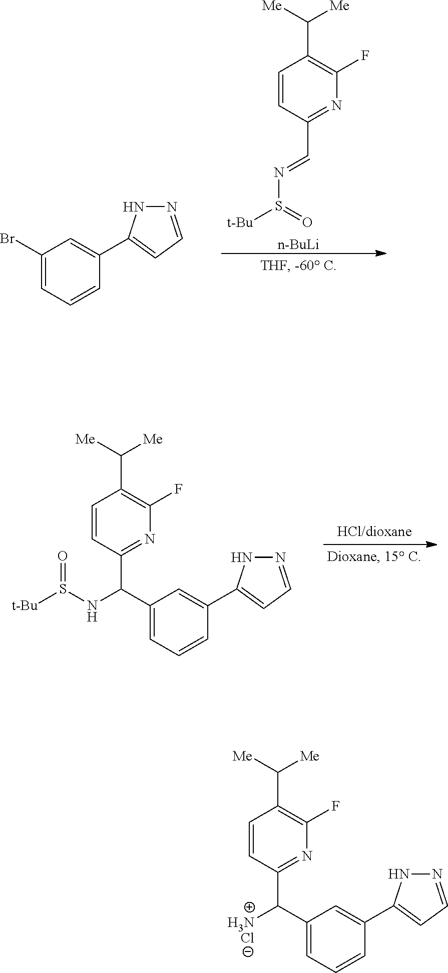

Step a: To a solution of 5-(3-bromophenyl)-1H-pyrazole (408 mg, 1.83 mmol, 1.5 eq) in THF (3 mL) at −60° C. under N2 was added n-BuLi (2.5 M, 1.22 mL, 2.5 eq) in a dropwise manner. Once this addition was complete, (E)-N-((6-fluoro-5-isopropylpyridin-2-yl)methylene)-2-methylpropane-2-sulfinamide (330 mg, 1.22 mmol, 1 eq) in THF (2 mL) was added in a dropwise manner. The resulting mixture was stirred at −60° C. for 2 h. The reaction mixture was then poured into ice-water (30 mL) and stirred for 2 min. The resulting biphasic mixture was then extracted with ethyl acetate (3×20 mL). The combined organic extracts were washed with brine (30 mL), dried over anhydrous Na₂SO₄, filtered, and concentrated under reduced pressure. The crude residue obtained was purified by column chromatography to give N-((3-(1H-pyrazol-5-yl)phenyl)(6-fluoro-5-isopropylpyridin-2-yl)methyl)-2-methylpropane-2-sulfinamide. LC-MS (ESI): m/z: [M+H]⁺ calculated for C₂₂H₂₆FN₄OS: 415.2; found 415.2.

Step b: To a solution of N-((3-(1H-pyrazol-5-yl)phenyl)(6-fluoro-5-isopropylpyridin-2-yl)methyl)-2-methylpropane-2-sulfinamide (410 mg, 989 umol, 1 eq) in dioxane (2 mL) at 15° C. was added HCl/dioxane (4 mL) in a dropwise manner. The resulting mixture was stirred at 15° C. for 2 h. The reaction mixture was then concentrated under reduced pressure to give (3-(1H-pyrazol-5-yl)phenyl)(6-fluoro-5-isopropylpyridin-2-yl)methanaminium chloride. LC-MS (ESI): m/z: [M+H]⁺ calculated for C₁₈H₁₉FN₄: 311.2; found 311.2.

### Intermediate A-3: Synthesis of tert-butyl (2-(amino(4-isopropylphenyl)methyl)phenyl)carbamate

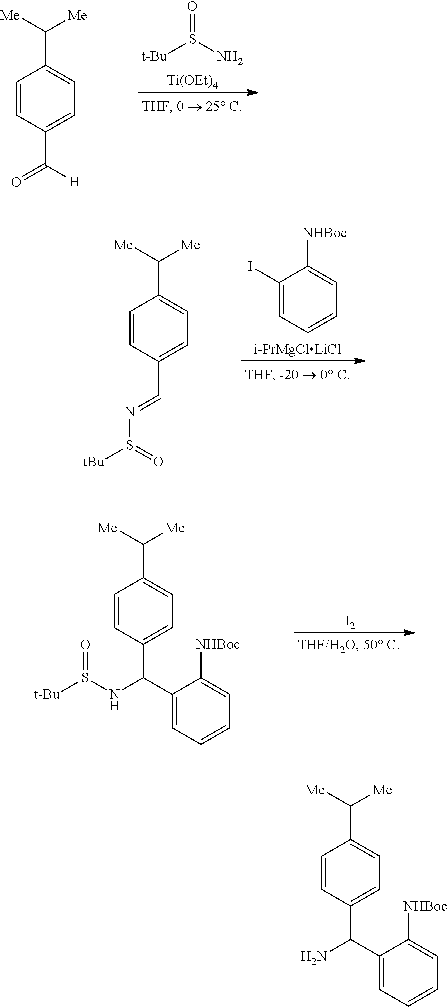

Step a: To a mixture of 4-isopropylbenzaldehyde (10.0 g, 67.4 mmol, 10.2 mL, 1 eq) and 2-methylpropane-2-sulfinamide (9.00 g, 74.2 mmol, 1.1 eq) in dry THF (75 mL) at 0° C. under N2 was added Ti(OEt)₄ (30.7 g, 134 mmol, 2 eq) in one portion. The reaction mixture was then degassed and charged with N₂ three times. The reaction mixture was then warmed to 25° C. and stirred for 4 h under N₂. The reaction was then cooled to 0° C. and quenched by adding H₂O (150 mL) and stirring for 20 min. The reaction mixture was then filtered, and the filter cake was washed with ethyl acetate (2×100 mL). The filtrate was then extracted with ethyl acetate (2×100 mL). The combined organic extracts were then washed with saturated aqueous NH₄Cl (100 mL), brine (70 mL), dried over anhydrous Na₂SO₄, and filtered, and the filtrate was concentrated under reduced pressure. The crude residue obtained was purified by column chromatography to give (E)-N-(4-isopropylbenzylidene)-2-methylpropane-2-sulfinamide. LC-MS (ESI): m/z: [M+H]⁺ calculated for C₁₄H₂₁NOS: 252.1; found 252.1.

Step b: To a solution of tert-butyl (2-iodophenyl)carbamate (25.4 g, 79.6 mmol, 2 eq) in THF (200 mL) at 0° C. was added i-PrMgCl·LiCl (107 mL, 1.3 M, 3.5 eq) in a dropwise manner over 30 min. After the addition was complete, the resulting mixture was stirred at 0° C. for 2 h. The reaction mixture was then cooled to −20° C. before (E)-N-(4-isopropylbenzylidene)-2-methylpropane-2-sulfinamide (10 g, 39.8 mmol, 1 eq) in THF (200 mL) was added in a dropwise manner. The resulting mixture was then stirred at −20° C. for 3 h. The reaction mixture was then warmed to 0° C. and quenched by addition water (100 mL). The resulting biphasic mixture was extracted with ethyl acetate (2×250 mL). The combined organic extracts were washed with brine (2×25 mL), dried over anhydrous Na₂SO₄, and filtered, and the filtrate was concentrated under reduced pressure. The crude residue obtained was purified by column chromatography to give tert-butyl (2-(((tert-butylsulfinyl)amino)(4-isopropylphenyl)methyl)phenyl)carbamate. LC-MS (ESI): m/z: [M+H]⁺ calculated for C₂₅H₃₆N₂O₃S: 445.2; found 445.4.

Step c: A mixture of tert-butyl (2-(((tert-butylsulfinyl)amino)(4-isopropylphenyl)methyl)phenyl)carbamate (15 g, 33.7 mmol, 1 eq) and 12 (6.85 g, 26.9 mmol, 0.8 eq) in THE (150 mL) and H₂O (30 mL) was degassed and purged with N₂, and then the mixture was warmed to 50° C. and stirred for 2 h under N₂ atmosphere. The reaction mixture was then cooled to 0° C. and quenched by addition of water (100 mL). The resulting biphasic mixture was extracted with ethyl acetate (2×250 mL). The combined organic extracts were washed with brine (2×25 mL), dried over anhydrous Na₂SO₄, and filtered, and the filtrate was concentrated under reduced pressure. The crude residue obtained was purified by column chromatography to give tert-butyl (2-(amino(4-isopropylphenyl)methyl)phenyl)carbamate. LC-MS (ESI): m/z: [M+H]⁺ calculated for C₂₁H₂₈N₂O₂: 341.2; found 341.4.

### Intermediate A-4: Synthesis of(S)-(3-fluoro-4-isopropylphenyl)(phenyl)methanaminium chloride

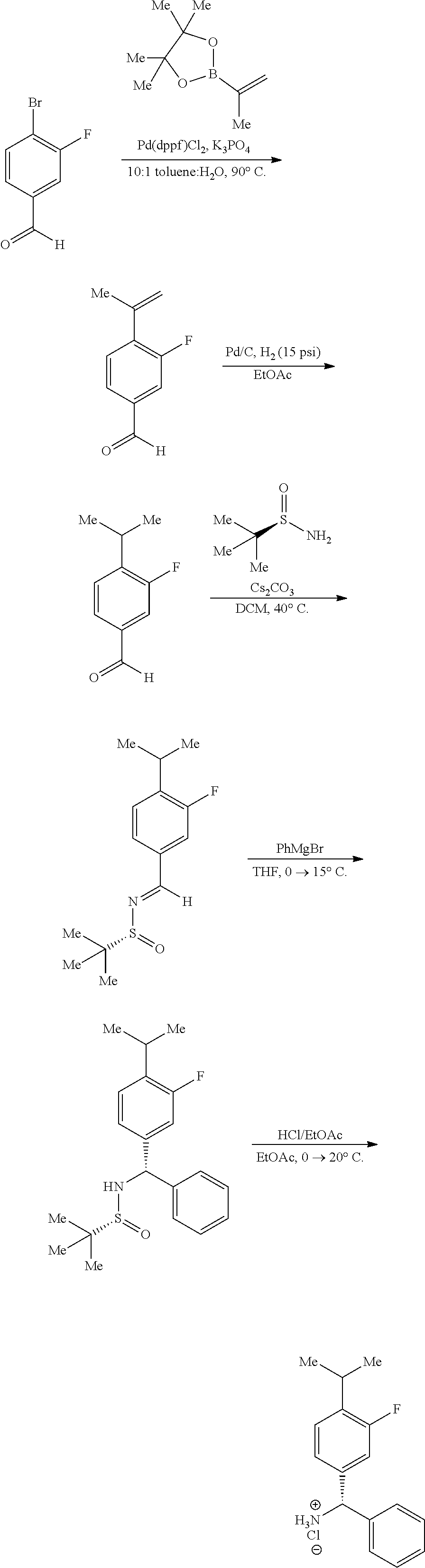

Step a: To a mixture of 4-bromo-3-fluoro-benzaldehyde (200 g, 985 mmol, 1.00 eq) and 2-isopropenyl-4,4,5,5-tetramethyl-1,3,2-dioxaborolane (215 g, 1.28 mol, 1.30 eq) in toluene (3.70 L) and H₂O (410 mL) at 25° C. under N₂ was added Pd(dppf)Cl₂ (36.0 g, 49.3 mmol, 0.05 eq) and K₃PO₄ (418 g, 1.97 mol, 2.00 eq). The mixture was warmed to 90° C. and stirred for 12 h. The reaction mixture was then filtered, and the filtrate was concentrated under reduced pressure. The resulting crude residue was purified by column chromatography to give 3-fluoro-4-isopropenyl-benzaldehyde. The compound was carried forward to the next step without further characterization.

Step b: To a solution of 3-fluoro-4-isopropenyl-benzaldehyde (124 g, 755 mmol, 1.00 eq) in EtOAc (1.20 L) under N₂ was added Pd/C (85.0 g, 10 wt. %). The suspension was degassed and purged with H₂ several times. The mixture was stirred at 25° C. under H₂ (15 psi) for 1 h. The reaction mixture was then filtered, and the filtrate was concentrated under reduced pressure. The resulting crude residue was purified by column chromatography to give 3-fluoro-4-isopropyl-benzaldehyde. The compound was carried forward to the next step without further characterization.

Step c: To a mixture of 3-fluoro-4-isopropyl-benzaldehyde (80.0 g, 481 mmol, 1.00 eq) and (R)-2-methylpropane-2-sulfinamide (64.2 g, 523 mmol, 1.10 eq) in DCM (450 mL) at 25° C. was added Cs₂CO₃ (173 g, 530 mmol, 1.10 eq). The mixture was warmed to 40° C. and stirred for 16 h. The reaction mixture was filtered, and the filtrate was concentrated under reduced pressure.

The residue was purified by column chromatography to obtain (R,E)-N-(3-fluoro-4-isopropylbenzylidene)-2-methylpropane-2-sulfinamide. LC-MS (ESI): m/z: [M+H]⁺ calculated for C₁₄H₂₀FNOS: 270.1; found 270.0.

Step d: To a solution of (R,E)-N-(3-fluoro-4-isopropylbenzylidene)-2-methylpropane-2-sulfinamide (30.0 g, 111 mmol, 1.00 eq) in THE (400 mL) −65° C. under N₂ was added, dropwise, a solution of phenylmagnesium bromide (3 M in Et₂O, 55.7 mL, 1.50 eq) over a period of 30 min. The reaction mixture was stirred at −65° C. for 6 h, then warmed to 25° C. and stirred for an additional 6 h. The reaction mixture was quenched with saturated aqueous NH₄Cl (50 mL) and extracted with EtOAc (3×30 mL). The combined organic extracts were washed with water (3×30 mL), dried over anhydrous Na₂SO₄, filtered, and concentrated under reduced pressure. The resulting crude residue was purified by column chromatography to obtain (R)—N—((S)-(3-fluoro-4-isopropylphenyl)(phenyl)methyl)-2-methylpropane-2-sulfinamide. The compound was carried forward to the next step without further characterization.

Step e: To a mixture of (R)—N—((S)-(3-fluoro-4-isopropylphenyl)(phenyl)methyl)-2-methylpropane-2-sulfinamide (35.0 g, 101 mmol, 1.00 eq) in EtOAc (300 mL) at 25° C. was added HCl/EtOAc (4 M, 50.4 mL, 2.00 eq), and the mixture was stirred for 2 h. The reaction mixture was filtered and the solid so obtained was set aside. The filtrate was concentrated under reduced pressure and the resulting residue was combined with the previously obtained solid. The mixture was dissolved in MTBE (200 mL) and filtered, and the filtrate was concentrated under reduced pressure to give (S)-(3-fluoro-4-isopropylphenyl)(phenyl)methanaminium chloride.

### Intermediate A-5: Synthesis of (S)-(5-cyclopropyl-6-fluoropyridin-2-yl)(phenyl)methanaminium chloride

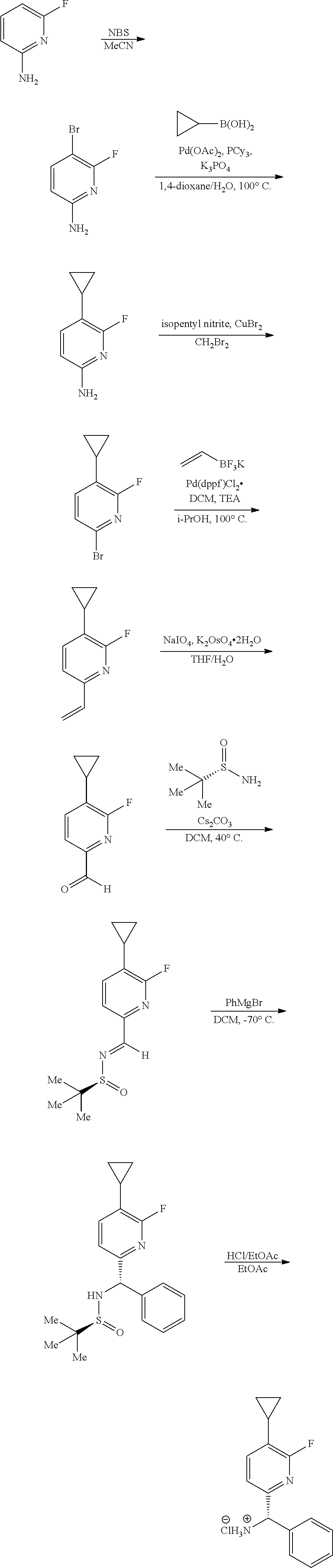

Step a: In four parallel reactions, 6-fluoropyridin-2-amine (125 g, 1.11 mol, 1 eq) in MeCN (1.2 L) at 0° C. under N₂ was treated with NBS (209 g, 1.17 mmol, 1.05 eq) in MeCN (1.2 L). The reaction mixtures were stirred at 20° C. for 2 h. The four parallel reactions were combined, and the resulting mixture was concentrated under reduced pressure. The resulting crude residue was purified by column chromatography to give 5-bromo-6-fluoropyridin-2-amine. LC-MS (ESI): m/z: [M+H]⁺ calculated for C₅H₄BrFN₂: 190.9; found 191.0.

Step b: To a mixture of 5-bromo-6-fluoropyridin-2-amine (200 g, 1.04 mol, 1 eq) and cyclopropylboronic acid (226 g, 2.63 mol, 2.5 eq) in 1,4-dioxane (2 L) and H₂O (200 mL) under N₂ were added K₃PO₄ (666 g, 3.14 mol, 3 eq), PCy₃ (58.6 g, 209 mmol, 0.2 eq), and Pd(OAc)2 (11.7 g, 52.3 mmol, 0.05 eq). The system was then degassed and charged with nitrogen three times. The reaction mixture was warmed to 100° C. and stirred for 12 h. The reaction mixture was then cooled to room temperature and filtered through Celite. The resulting filtrate was diluted with H₂O (2 L) and then extracted with EtOAc (3×500 mL). The combined organic extracts were washed with brine (2×300 mL), dried over anhydrous Na₂SO₄, filtered, and concentrated under reduced pressure. The crude residue obtained was purified by column chromatography to give 5-cyclopropyl-6-fluoropyridin-2-amine. LC-MS (ESI): m/z: [M+H]⁺ calculated for C₈H₉FN₂: 153.1; found 153.0.

Step c: To a mixture of 5-cyclopropyl-6-fluoropyridin-2-amine (120 g, 788 mmol, 1 eq) in dibromomethane (564 mL) under N₂ was added isopentyl nitrite (110 g, 946 mmol, 127 mL, 1.2 eq). To the resulting mixture was added CuBr₂ (211 g, 946 mmol, 44.3 mL, 1.2 eq) over 0.5 h. The final mixture was then degassed and charged with nitrogen three times before stirring at 20° C. for 16 h. The reaction mixture was then filtered, and the filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was purified by column chromatography to give 6-bromo-3-cyclopropyl-2-fluoropyridine. LC-MS (ESI): m/z: [M+H]⁺ calculated for C₈H₇BrFN: 216.0; found 216.1.

Step d: To a mixture of 6-bromo-3-cyclopropyl-2-fluoropyridine (90 g, 416 mmol, 1 eq) and trifluoro(vinyl)-λ4-borane, potassium salt (83.7 g, 624 mmol, 1.5 eq) in i-PrOH (900 mL) at 20° C. under N₂ was added TEA (126 g, 1.25 mol, 3 eq) and Pd(dppf)Cl₂·DCM (17 g, 20.8 mmol, 0.05 eq). The resulting mixture was degassed and charged with nitrogen three times. The reaction mixture was then warmed to 100° C. and stirred for 2 h. The reaction mixture was then cooled to room temperature and filtered. The filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was then purified by column chromatography to give 3-cyclopropyl-2-fluoro-6-vinylpyridine. LC-MS (ESI): m/z: [M+H]⁺ calculated for C₁₀H₁₀FN: 164.1; found 164.1.

Step e: To a mixture of 3-cyclopropyl-2-fluoro-6-vinylpyridine (47 g, 288 mmol, 1 eq) in THE (800 mL) and H₂O (160 mL) at 20° C. under N₂ was added NaIO₄ (246 g, 1.15 mol, 4 eq) and K₂OsO₄·2H₂O (2.12 g, 5.76 mmol, 0.02 eq). The resulting mixture was degassed and charged with nitrogen three times before stirring for 2 h. The reaction mixture was then filtered, and the filtrate was diluted with H₂O (500 mL), and extracted with EtOAc (3×300 mL). The combined organic extracts were washed with brine (300 mL), dried over anhydrous Na₂SO₄, filtered, and concentrated under reduced pressure. The resulting crude residue was purified by column chromatography to give 5-cyclopropyl-6-fluoropicolinaldehyde. LC-MS (ESI): m/z: [M+H]⁺ calculated for C₉H₈FNO: 166.1; found 166.2.

Step f: To a mixture of 5-cyclopropyl-6-fluoropicolinaldehyde (38 g, 230 mmol, 1 eq) and (S)-2-methylpropane-2-sulfinamide (30.6 g, 253 mmol, 1.1 eq) in DCM (200 mL) at 20° C. under N₂ was added Cs₂CO₃ (82.4 g, 253 mmol, 1.1 eq). The system was then degassed and charged with nitrogen three times. The resulting mixture was then warmed to 40° C. and stirred for 12 h. The reaction solution was then diluted with H₂O (300 mL) and extracted with DCM (3×200 mL). The combined organic extracts were washed with brine (200 mL), dried over anhydrous Na₂SO₄, filtered, and concentrated under reduced pressure. The resulting crude residue was then purified by column chromatography to give (S,E)-N-((5-cyclopropyl-6-fluoropyridin-2-yl)methylene)-2-methylpropane-2-sulfinamide. LC-MS (ESI): m/z: [M+H]⁺ calculated for C₁₃H₁₇FN₂OS: 269.1; found 269.2.

Step g: To a solution of (S,E)-N-((5-cyclopropyl-6-fluoropyridin-2-yl)methylene)-2-methylpropane-2-sulfinamide (58 g, 216 mmol, 1 eq) in dry DCM (600 mL) at −70° C. under nitrogen was added PhMgBr (3 M in Et₂O, 93.6 mL, 281 mmol, 1.3 eq) in a dropwise manner. The resulting reaction mixture was stirred at −70° C. for 1 h. The reaction mixture was then quenched with saturated aqueous NH₄Cl solution (500 mL), warmed to room temperature, and extracted with EtOAc (3×200 mL). The combined organic extracts were washed with brine (200 mL), dried over anhydrous Na₂SO₄, filtered, and concentrated under reduced pressure. The crude residue obtained was purified by column chromatography to give (S)—N—((S)-(5-cyclopropyl-6-fluoropyridin-2-yl)(phenyl)methyl)-2-methylpropane-2-sulfinamide. LC-MS (ESI): m/z: [M+H]⁺ calculated for C₁₉H₂₃FN₂OS: 347.2; found 347.3.

Step h: To a solution of (S)—N—((S)-(5-cyclopropyl-6-fluoropyridin-2-yl)(phenyl)methyl)-2-methylpropane-2-sulfinamide (74 g, 213 mmol, 1 eq) in EtOAc (100 mL) at 0° C. under N₂ was added HCl/EtOAc (4 M, 740 mL, 2940 mmol, 13.8 eq). The resulting mixture was then warmed 20° C. and stirred for 1 h. The reaction mixture was then concentrated under reduced pressure, and the crude residue obtained was triturated with MTBE (500 mL). The resulting solid was collected by filtration and dried under reduced pressure to give (S)-(5-cyclopropyl-6-fluoropyridin-2-yl)(phenyl)methanaminium chloride. LC-MS (ESI): m/z: [M+H]⁺ calculated for C₁₅H₁₅FN₂: 243.1; found 243.2.

### Intermediate A-6: Synthesis of (R)-(4-isopropylphenyl)(2-(1-methylpiperidine-4-carboxamido)phenyl)methanaminium chloride

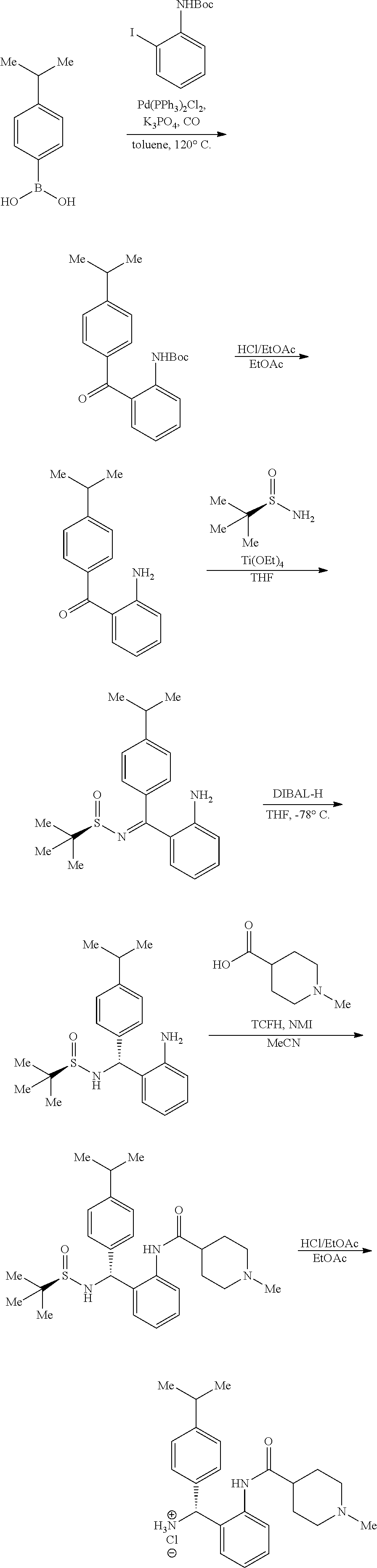

Step a: To a solution of tert-butyl (2-iodophenyl)carbamate (15 g, 47.0 mmol, 1 eq) and (4-isopropylphenyl)boronic acid (9.25 g, 56.4 mmol, 1.2 eq) in toluene (100 mL) was added K₂CO₃ (19.5 g, 141 mmol, 3 eq) and Pd(PPh₃)₂Cl₂ (1.65 g, 2.35 mmol, 0.05 eq) under N₂. The resulting mixture was then degassed and purged with CO. The resulting mixture was warmed to 120° C. and stirred for 10 h under CO (15 psi). The reaction mixture was then cooled to room temperature and poured into ice water (100 mL). The resulting biphasic mixture was extracted with EtOAc (3×100 mL). The combined organic extracts were dried over anhydrous Na₂SO₄, filtered, and concentrated under reduced pressure. The crude residue obtained was purified by column chromatography to give tert-butyl (2-(4-isopropylbenzoyl)phenyl)carbamate.

Step b: To a solution of tert-butyl (2-(4-isopropylbenzoyl)phenyl)carbamate (10 g, 29.4 mmol, 1 eq) in EtOAc (20 mL) was added HCl/EtOAc (4 M, 100 mL), and the resulting mixture was stirred at 20° C. for 16 h. The reaction mixture was then diluted with water (50 mL), and the pH was adjusted to 8 with saturated aq. NaHCO₃. The resulting biphasic mixture was extracted with ethyl acetate (3×50 mL). The combined organic extracts were washed with brine (20 mL), dried over anhydrous Na₂SO₄, filtered, and concentrated under reduced pressure to give (2-aminophenyl)(4-isopropylphenyl)methanone. LC-MS (ESI): m/z: [M+H]⁺ calculated for C₁₆H₁₇N₂O: 240.1; found 240.1.

Step c: To a solution of (2-aminophenyl)-(4-isopropylphenyl)methanone (7.3 g, 30.5 mmol, 1 eq) in THE (120 mL) was added (R)-2-methylpropane-2-sulfinamide (4.07 g, 33.5 mmol, 1.1 eq) and Ti(OEt)4 (13.9 g, 61.0 mmol, 2 eq), sequentially. The reaction was warmed to 80° C. and stirred for 16 h. The reaction mixture was then cooled to room temperature and quenched with water (50 mL). The resulting biphasic mixture was exacted with ethyl acetate (2×50 mL), and the combined organic extracts were washed with brine (80 mL), dried over anhydrous Na₂SO₄, filtered, and concentrated under reduced pressure. The crude residue obtained was purified by column chromatography to obtain (R,E)-N-((2-aminophenyl)(4-isopropylphenyl)methylene)-2-methylpropane-2-sulfinamide. LC-MS (ESI): m/z: [M+H]⁺ calculated for C₂₀H₂₆N₂OS: 343.2; found 343.1.

Step d: To a solution of (R,E)-N-((2-aminophenyl)(4-isopropylphenyl)methylene)-2-methylpropane-2-sulfinamide (2.5 g, 7.30 mmol, 1 eq) in THE (25 mL) at −78° C. was added DIBAL-H (20.4 mL, 1 M, 2.8 eq). The resulting mixture was stirred at −78° C. for 2 h. The reaction mixture was then diluted with brine (50 mL), and the resulting biphasic mixture was extracted with EtOAC (3×40 mL). The combined organic extracts were washed with brine (20 mL), dried over anhydrous Na₂SO₄, filtered, and concentrated under reduced pressure. The crude residue obtained was purified by column chromatography to give (R)—N—((R)-(2-aminophenyl)(4-isopropylphenyl)methyl)-2-methylpropane-2-sulfinamide. LC-MS (ESI): m/z: [M+Na]⁺ calculated for C₂₀H₂₈N₂OS: 367.2; found 367.1.

Step e: To a solution of (R)—N—((R)-(2-aminophenyl)(4-isopropylphenyl)methyl)-2-methylpropane-2-sulfinamide (250 mg, 725 μmol, 1 eq) in CH₃CN (2 mL) at −20° C. was added N-methylimidazole (148 mg, 1.81 mmol, 2.5 eq), 1-methylpiperidine-4-carboxylic acid (114 mg, 798 μmol, 1.1 eq), and chloro-N,N,N′,N′-tetramethylformamidinium hexafluorophosphate (244 mg, 870 μmol, 1.2 eq). The resulting mixture was warmed to 0° C. and stirred for 3 h. The reaction mixture was then diluted with water, extracted with EtOAc 30 mL (3×10 mL), dried over anhydrous Na₂SO₄, filtered, and concentrated under reduced pressure. The crude residue obtained was purified by column chromatography to give N-(2-((R)-(((R)-tert-butylsulfinyl)amino)(4-isopropylphenyl)methyl)phenyl)-1-methylpiperidine-4-carboxamide. LC-MS (ESI): m/z: [M+H]⁺ calculated for C₂₇H₃₉N₃O₂S: 470.3; found 470.3.

Step f: To a solution of N-(2-((R)-(((R)-tert-butylsulfinyl)amino)(4-isopropylphenyl)methyl)phenyl)-1-methylpiperidine-4-carboxamide in EtOAc (2 mL) at 0° C. was added HCl/EtOAc (2 mL). The mixture was then warmed to 15° C. and stirred for 1 h. The reaction mixture was then concentrated under reduced pressure. The crude residue obtained was triturated with MTBE at 15° C. for 30 min to obtain (R)-(4-isopropylphenyl)(2-(1-methylpiperidine-4-carboxamido)phenyl)methanaminium chloride. LC-MS (ESI): m/z: [M+H]⁺ calculated for C₂₃H₃₁N₃: 366.3; found 366.3.

The following compounds in Table B-1 were synthesized using procedures similar to Intermediates A-1 through A-6 using the appropriate starting materials and reagents.

TABLE B-1

| Inter- | | | Exact | LCMS, |
|---|---|---|---|---|
| mediate | | | mass | Found |
| No. | Structure | IUPAC | (g/mol) | [M + H]⁺ |
| B-1-1 | 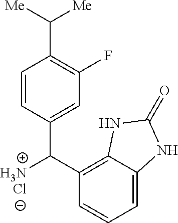 | (3-fluoro-4- isopropylphenyl)(2- oxo-2,3-dihydro-1H- benzo[d]imidazol-4- yl)methanaminium chloride | 299.1 | 282.9 [M − NH₃]⁺ |
| B-1-2 | 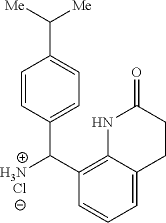 | (4-isopropylphenyl)(2- oxo-1,2,3,4- tetrahydroquinolin-8- yl)methanaminium chloride | 294.2 | 278.3 [M − NH₃]⁺ |
| B-1-3 | 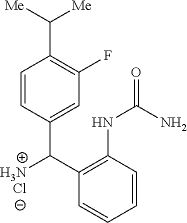 | (3-fluoro-4- isopropylphenyl)(2- ureidophenyl) methanaminium chloride | 301.2 | |
| B-1-4 | 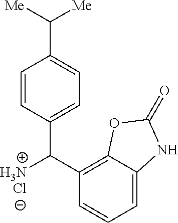 | (4-isopropylphenyl)(2- oxo-2,3- dihydrobenzo[d]oxazol- 7-yl)methanaminium chloride | 282.1 | 266.2 [M − NH₃]⁺ |
| B-1-5 | 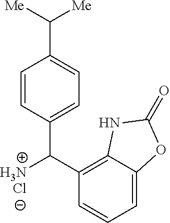 | (4-isopropylphenyl)(2- oxo-2,3- dihydrobenzo[d]oxazol- 4-yl)methanaminium chloride | 282.1 | 266.1 [M − NH₃]⁺ |
| B-1-6 | 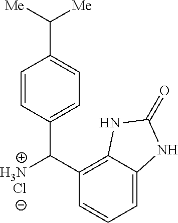 | (4-isopropylphenyl)(2- oxo-2,3-dihydro-1H- benzo[d]imidazol-4- yl)methanaminium chloride | 281.1 | 265.1 [M − NH₃]⁺ |
| B-1-7 | 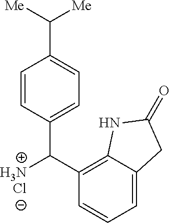 | (4-isopropylphenyl)(2- oxoindolin-7- yl)methanaminium chloride | 280.2 | 264.3 [M − NH₃]⁺ |
| B-1-8 | 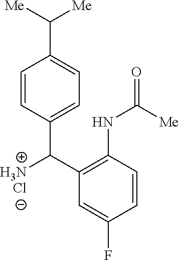 | (2-acetamido-5- fluorophenyl)(4- isopropylphenyl) methanaminium chloride | 300.2 | 301.2 |
| B-1-9 | 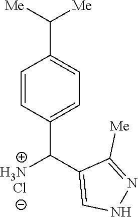 | (4-isopropylphenyl)(3- methyl-1H-pyrazol-4- yl)methanaminium chloride | 229.2 | 213.1 [M − NH₃]⁺ |
| B-1-10 | 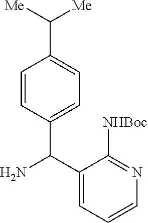 [SMILES: CC(C)c1ccc(C(N)c2cccnc2NC(=O)OC(C)(C)C)cc1] | tert-butyl (3-(amino(4- isopropylphenyl) methyl)pyridin-2- yl)carbamate | 341.2 | 342.2 |
| B-1-11 | 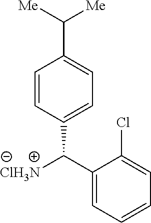 | (R)-(2- chlorophenyl)(4- isopropylphenyl) methanaminium chloride | 259.1 | 243.2 [M − NH₃]⁺ |
| B-1-12 | 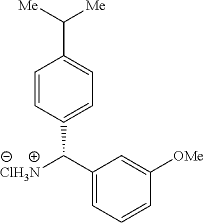 | (R)-(4- isopropylphenyl)(3- methoxyphenyl) methanaminium chloride | 255.2 | 239.1 [M − NH₃]⁺ |
| B-1-13 | 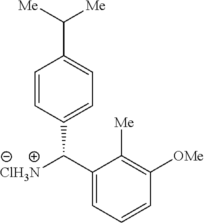 | (R)-(4- isopropylphenyl)(3- methoxy-2- methylphenyl) methanaminium chloride | 269.2 | 253.3 [M − NH₃]⁺ |
| B-1-14 | 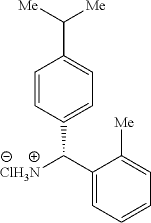 | (R)-(4- isopropylphenyl)(o- tolyl)methanaminium chloride | 239.2 | 223.2 [M − NH₃]⁺ |
| B-1-15 | 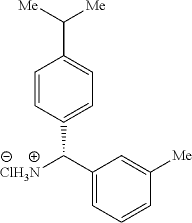 | (R)-(4- isopropylphenyl)(m- tolyl)methanaminium chloride | 239.2 | 223.2 [M − NH₃]⁺ |
| B-1-16 | 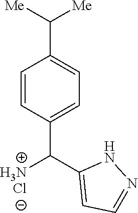 | (4- isopropylphenyl)(1H- pyrazol-5- yl)methanaminium chloride | 215.1 | 199.1 [M − NH₃]⁺ |
| B-1-17 | 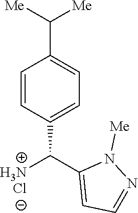 | (R)-(4- isopropylphenyl)(1- methyl-1H-pyrazol-5- yl)methanaminium chloride | 229.1 | 230.1 |
| B-1-18 | 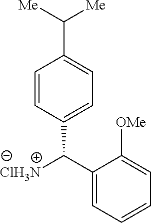 | (R)-(4- isopropylphenyl)(2- methoxyphenyl) methanaminium chloride | 255.2 | 239.1 [M − NH₃]⁺ |
| B-1-19 | 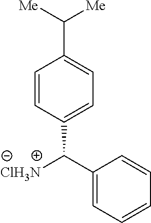 | (S)-(4- isopropylphenyl) (phenyl) methanaminium chloride | 225.1 | 209.1 [M − NH₃]⁺ |
| B-1-20 | 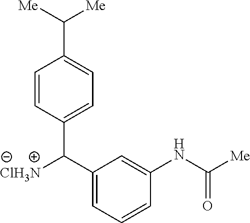 | (3- acetamidophenyl)(4- isopropylphenyl) methanaminium chloride | 282.2 | 266.1 [M − NH₃]⁺ |
| B-1-21 | 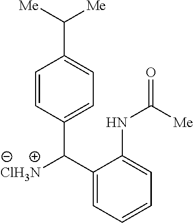 | (2- acetamidophenyl)(4- isopropylphenyl) methanaminium chloride | 282.2 | 266.2 [M − NH₃]⁺ |
| B-1-22 | 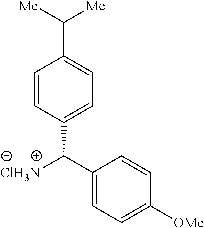 | (R)-(4- isopropylphenyl)(4- methoxyphenyl) methanaminium chloride | 255.2 | 239.1 [M − NH₃]⁺ |
| B-1-23 | 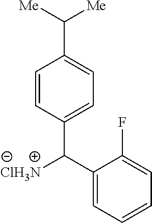 | (2-fluorophenyl)(4- isopropylphenyl) methanaminium chloride | 243.1 | 227.1 [M − NH₃]⁺ |
| B-1-24 | 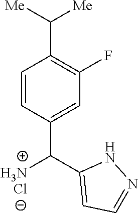 | (3-fluoro-4- isopropylphenyl)(1H- pyrazol-5- yl)methanaminium chloride | 233.1 | 217.1 [M − NH₃]⁺ |
| B-1-25 | 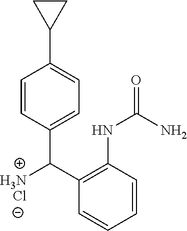 | (4- cyclopropylphenyl)(2- ureidophenyl) methanaminium chloride | 281.1 | 282.1 |
| B-1-26 | 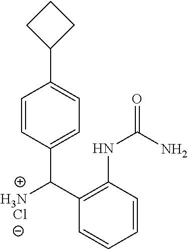 | (4- cyclobutylphenyl)(2- ureidophenyl) methanaminium chloride | 295.2 | 296.2 |
| B-1-27 | 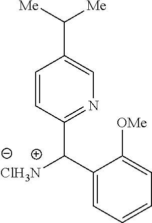 | (5-isopropylpyridin-2- yl)(2- methoxyphenyl) methanaminium chloride | 256.1 | 257.1 |
| B-1-28 | 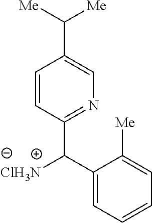 | (5-isopropylpyridin-2- yl)(o- tolyl)methanaminium chloride | 240.2 | 241.1 |
| B-1-29 | 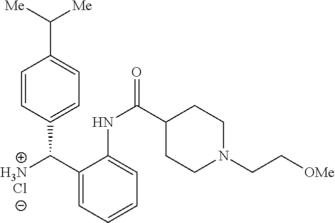 | (R)-(4- isopropylphenyl)(2-(1- (2- methoxyethyl) piperidine-4- carboxamido)phenyl) methanaminium chloride | 409.3 | 410.3 |
| B-1-30 | 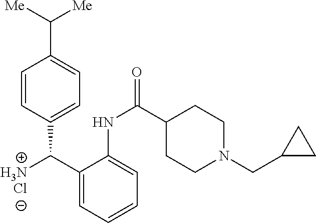 | (R)-(2-(1- (cyclopropylmethyl) piperidine-4- carboxamido)phenyl) (4-isopropylphenyl) methanaminium chloride | 405.3 | 406.2 |
| B-1-31 | 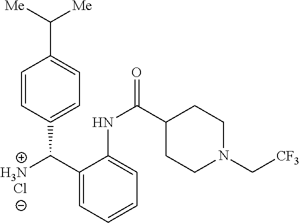 | (R)-(4- isopropylphenyl)(2-(1- (2,2,2- trifluoroethyl) piperidine-4- carboxamido)phenyl) methanaminium chloride | 433.2 | 434.2 |

### Example S-1: Synthesis of (1R,2S)-2-fluoro-N—((S or R)-(6-fluoro-5-isopropylpyridin-2-yl)(1H-indazol-6-yl)methyl)cyclopropane-1-carboxamide (Compound 3)

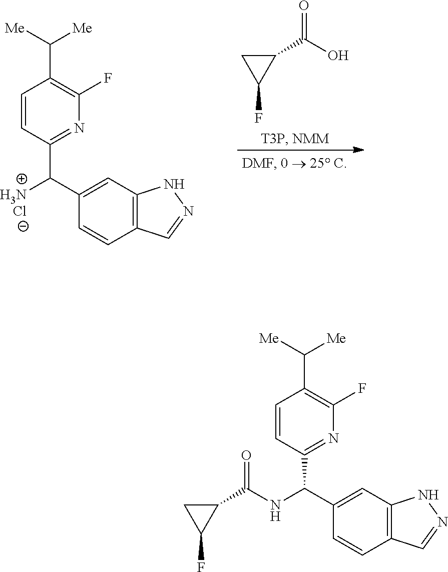

Step a: To a solution of (6-fluoro-5-isopropylpyridin-2-yl)(1H-indazol-6-yl)methanaminium chloride (150 mg, 468 μmol, 1 eq) in DMF (2 mL) at 0° C. was added (1R,2S)-2-fluorocyclopropane-1-carboxylic acid (58 mg, 561 μmol, 1.2 eq), NMM (1.87 mmol, 206 μL, 4 eq), and T3P (595 mg, 935 μmol, 50% purity, 2 eq). The resulting mixture was warmed to 25° C. and stirred for 1 h. The reaction mixture was then diluted with water, and the resulting biphasic mixture was extracted with ethyl acetate (3×20 mL), dried over anhydrous Na₂SO₄, filtered, and concentrated under reduced pressure. The crude residue obtained was purified by prep-HPLC to give (1R,2S)-2-fluoro-N-((6-fluoro-5-isopropylpyridin-2-yl)(1H-indazol-6-yl)methyl)cyclopropane-1-carboxamide. This mixture of diastereomers was separated by chiral SFC (column: DAICEL CHIRALPAK AD) to give (1R,2S)-2-fluoro-N—((S or R)-(6-fluoro-5-isopropylpyridin-2-yl)(1H-indazol-6-yl)methyl)cyclopropane-1-carboxamide as the second eluting isomer. LC-MS (ESI): m/z: [M+H]⁺ calculated for C₂₀H₂₀F₂N₄O: 371.2; found 371.1.

### Example S-2: Synthesis of (R)—N-((2-aminophenyl)(4-isopropylphenyl)methyl)cyclopropanecarboxamide (Compound 64) and (R)—N-(2-(cyclopropanecarboxamido(4-isopropylphenyl)methyl)phenyl)-1-methylazetidine-3-carboxamide (Compound 26)

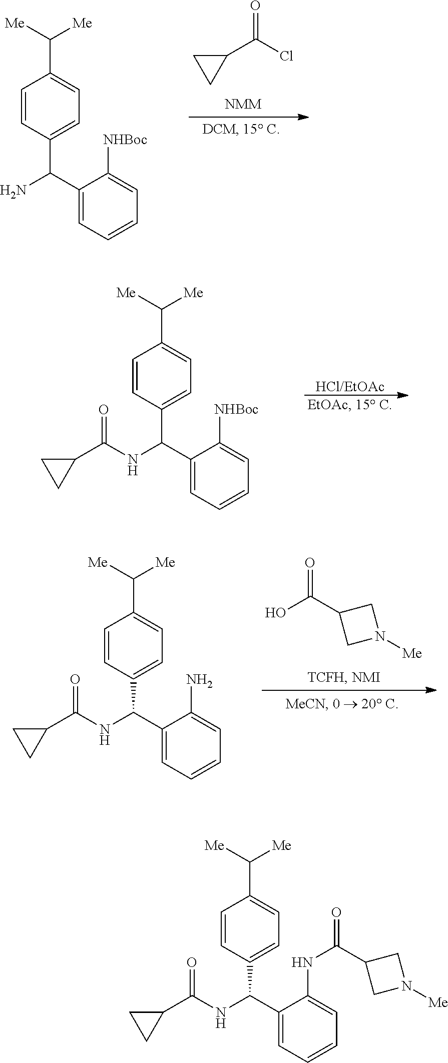

Step a: To a solution of tert-butyl (2-(amino(4-isopropylphenyl)methyl)phenyl)carbamate (4 g, 7.05 mmol, 60% purity, 1 eq) in DCM (100 mL) at 15° C. was added NMM (14.1 mmol, 1.55 mL, 2 eq) and cyclopropanecarbonyl chloride (884 mg, 8.46 mmol, 1.2 eq). The resulting mixture was stirred at 15° C. for 1 h. The reaction mixture was then poured into ice-water (50 mL) and stirred for 3 min. The resulting biphasic mixture was extracted with ethyl acetate (3×50 mL). The combined organic extracts were washed with brine (50 mL), dried over anhydrous Na₂SO₄, filtered, and concentrated under reduced pressure. The crude residue obtained was purified by column chromatography to give tert-butyl (2-(cyclopropanecarboxamido(4-isopropylphenyl)methyl)phenyl)carbamate. LC-MS (ESI): m/z: [M+H]⁺ calculated for C₂₅H₃₂N₂O₃: 409.2; found 409.3.

Step b: To a solution of tert-butyl (2-(cyclopropanecarboxamido(4-isopropylphenyl)methyl)phenyl)carbamate (3.5 g, 8.57 mmol, 1 eq) in EtOAc (30 mL) at 15° C. was added HCl/EtOAc (35 mL). The resulting mixture was stirred at 15° C. for 16 h. The reaction mixture was then concentrated under reduced pressure to give N-((2-aminophenyl)(4-isopropylphenyl)methyl)cyclopropanecarboxamide. This mixture of enantiomers was separated by chiral SFC ((s, s) WHELK-01, First Eluting Isomer) to give (R)—N-((2-aminophenyl)(4-isopropylphenyl)methyl)cyclopropanecarboxamide as the first eluting isomer. LC-MS (ESI): m/z: [M+H]⁺ calculated for C₂₀H₂₄N₂O: 309.2; found 309.4.

Step c: To a solution of (R)—N-((2-aminophenyl)(4-isopropylphenyl)methyl)cyclopropanecarboxamide (60 mg, 0.194 mmol, 1 eq) in MeCN (2 mL) was added 1-methylazetidine-3-carboxylic acid (44 mg, 0.389 mmol, 2 eq). The resulting mixture was cooled to 0° C. before N-methylimidazole (39.9 mg, 0.486 mmol, 2.5 eq) and TCFH (17.5 mg, 0.486 mmol, 2.5 eq) were added sequentially. The resulting mixture was warmed to 20° C. and stirred for 2 h. The reaction mixture was then quenched by addition of ice-water (10 mL), and the resulting biphasic mixture was extracted with EtOAc (3×10 mL). The combined organic extracts were washed with brine (10 mL), dried over anhydrous Na₂SO₄, filtered, and concentrated under reduced pressure. The crude residue obtained was purified by prep-HPLC to give (R)—N-(2-(cyclopropanecarboxamido(4-isopropylphenyl)methyl)phenyl)-1-methylazetidine-3-carboxamide. LC-MS (ESI): m/z: [M+H]⁺ calculated for C₂₅H₃₁N₃O₂: 406.2; found 406.2.

### Example S-3: Synthesis of (R)—N-(2-(cyclopropanecarboxamido(4-isopropylphenyl)methyl)phenyl)azetidine-3-carboxamide (Compound 48) and (R)-1-acetyl-N-(2-(cyclopropanecarboxamido(4-isopropylphenyl)methyl)phenyl)azetidine-3-carboxamide (Compound 37)

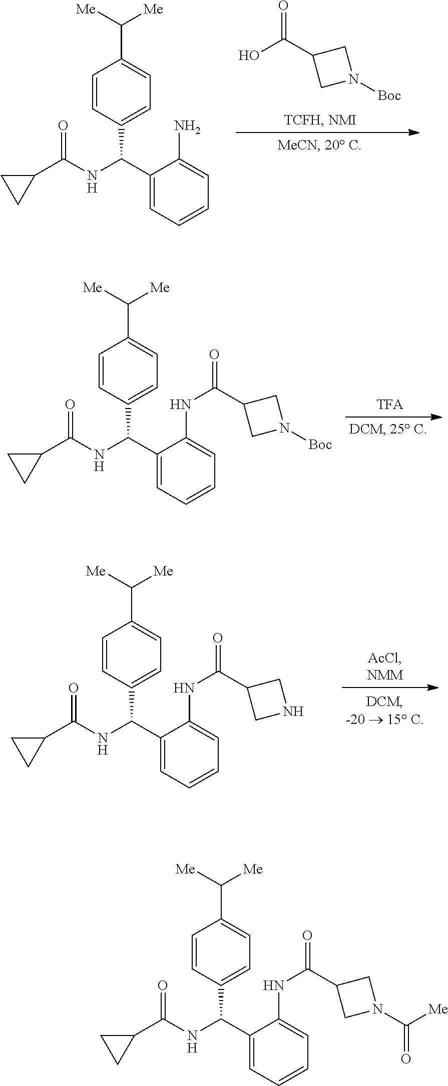 [SMILES: CC(C)c1ccc([C@@H](NC(=O)C2CC2)c2ccccc2N)cc1 ; CC(C)(C)OC(=O)N1CC(C(=O)O)C1 ; CC(C)c1ccc([C@@H](NC(=O)C2CC2)c2ccccc2NC(=O)C2CN(C(=O)OC(C)(C)C)C2)cc1 ; CC(C)c1ccc([C@@H](NC(=O)C2CC2)c2ccccc2NC(=O)C2CNC2)cc1 ; CC(=O)N1CC(C(=O)Nc2ccccc2[C@H](NC(=O)C2CC2)c2ccc(C(C)C)cc2)C1]

Step a: To a mixture of (R—N-((2-aminophenyl)4-isopropylphenyl)methyl)cyclopropanecarboxamide (150 mg, 486 μmol, 1 eq), N-methylimidazole (120 mg, 1.46 mmol, 3 eq) and 1-(tert-butoxycarbonyl)azetidine-3-carboxylic acid (108 mg, 535 μmol, 1.1 eq) in MeCN (5 mL) at 20° C. under N₂ was added TCFH (136 mg, 486 umol, 1 eq). The resulting mixture was then stirred at 20° C. for 1 h. The reaction mixture was then poured into ice-water (10 mL), and the resulting biphasic mixture was extracted with EtOAc (2×20 mL). The combined organic extracts were washed with brine (100 mL), dried over anhydrous Na₂SO₄, filtered, and concentrated under reduced pressure. The crude residue obtained was purified by column chromatography to give tert-butyl (R)-3-((2-(cyclopropanecarboxamido(4-isopropylphenyl)methyl)phenyl)carbamoyl)azetidine-1-carboxylate. LC-MS (ESI): m/z: [M+H]⁺ calculated for C₂₉H₃₇N₃O₄: 492.3; found 492.3.

Step b: A mixture of tert-butyl (R)-3-((2-(cyclopropanecarboxamido(4-isopropylphenyl)methyl)phenyl)carbamoyl)azetidine-1-carboxylate (160 mg, 325 μmol, 1 eq) in DCM (3 mL) at 25° C. was added TFA (1.54 g, 13.5 mmol, 1.00 mL, 41.5 eq), and the resulting mixture was stirred at 25° C. for 30 min. The reaction mixture was then diluted with H₂O (10 mL), and the resulting biphasic mixture was extracted with EtOAc (2×20 mL). The combined organic extracts were washed with brine (10 mL), dried over anhydrous Na₂SO₄, filtered, and concentrated under reduced pressure. The crude residue obtained was purified by prep-HPLC to give (R)—N-(2-(cyclopropanecarboxamido(4-isopropylphenyl)methyl)phenyl)azetidine-3-carboxamide. LC-MS (ESI): m/z: [M+H]⁺ calculated for C₂₄H₂₉N₃O₂: 392.2; found 392.4.

Step c: To a mixture of (R)—N-(2-(cyclopropanecarboxamido(4-isopropylphenyl)methyl)phenyl)azetidine-3-carboxamide (10 mg, 25.5 μmol, 1 eq) and methylmorpholine (5.17 mg, 51.1 μmol, 2 eq) in DCM (1 mL) at −20° C. under N₂ was added acetyl chloride (2.01 mg, 25.5 μmol, 1 eq) in a dropwise manner. The resulting mixture was allowed to warm to 15° C. and stir for 1 h. The reaction mixture was then cooled to 0° C. and quenched by addition of H₂O (5 mL). The resulting biphasic mixture was stirred for 5 min before it was extracted with ethyl acetate (2×10 mL). The combined organic extracts were washed with brine (5 mL), dried over anhydrous Na₂SO₄, filtered, and concentrated under reduced pressure. The crude residue was purified by prep-HPLC to give (R)-1-acetyl-N-(2-(cyclopropanecarboxamido(4-isopropylphenyl)methyl)phenyl)azetidine-3-carboxamide. LC-MS (ESI): m/z: [M+H]⁺ calculated for C₂₆H₃₁N₃O₃: 434.2; found 434.2.

### Example S-4: Synthesis of (R or S)—N-((4-isopropylphenyl)(2-(oxazol-2-ylamino)phenyl)methyl)cyclopropanecarboxamide (Compound 31)

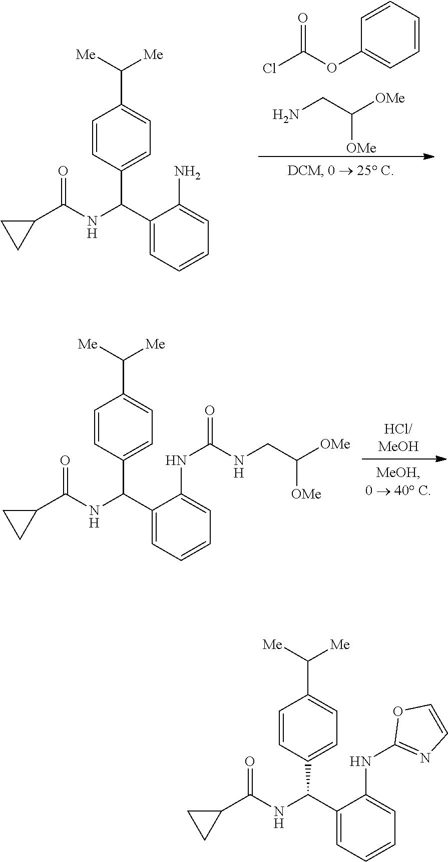

Step a: To a solution of N-((2-aminophenyl)(4-isopropylphenyl)methyl)cyclopropanecarboxamide (500 mg, 1.62 mmol, 1.00 eq) in DCM (30.0 mL) at 0° C. was added phenyl carbonochloridate (305 mg, 1.95 mmol, 1.20 eq) and triethylamine (820 mg, 8.11 mmol, 5.00 eq). The resulting mixture was warmed to 25° C. and stirred for 1 h. 2,2-dimethoxyethan-1-amine (341 mg, 3.24 mmol, 2.00 eq) was then added to the reaction mixture, and the resulting mixture was at 25° C. for 11 h. The reaction mixture was then diluted with H₂O (30 mL), and the resulting biphasic mixture was exacted with DCM (20 mL). The combined organic extracts were dried over anhydrous Na₂SO₄, filtered, and concentrated under reduced pressure. The crude residue obtained was purified by column chromatography to give N-((2-(3-(2,2-dimethoxyethyl)ureido)phenyl)(4-isopropylphenyl)methyl)cyclopropanecarboxamide. LC-MS (ESI): m/z: [M+H]⁺ calculated for C₂₅H₃₃N₃O₄: 440.2; found 440.3.

Step b: To a solution of N-((2-(3-(2,2-dimethoxyethyl)ureido)phenyl)(4-isopropylphenyl)methyl)cyclopropanecarboxamide (250 mg, 569 μmol, 1.00 eq) in MeOH (0.5 mL) at 0° C. was added HCl/MeOH (0.5 mL). The resulting mixture was warmed to 40° C. and stirred for 12 h. The reaction mixture was then concentrated under reduced pressure. The crude residue was then partitioned between water and DCM (30 mL). The organic layer was collected and washed with saturated aqueous NaHCO₃ (10 mL) and saturated aqueous NaCl (10 mL). The washed organic layer was then dried over anhydrous Na₂SO₄, filtered, and concentrated under reduced pressure. The crude residue obtained was purified by prep-HPLC to give N-((4-isopropylphenyl)(2-(oxazol-2-ylamino)phenyl)methyl)cyclopropanecarboxamide. This mixture of enantiomers was then separated using chiral SFC (column: DAICEL CHIRALPAK AD) to give (R or S)—N-((4-isopropylphenyl)(2-(oxazol-2-ylamino)phenyl)methyl)cyclopropanecarboxamide as the first eluting isomer. LC-MS (ESI): m/z: [M+H]⁺ calculated for C₂₃H₂₅N₃O₂: 376.2; found 376.2.

### Example S-5: Synthesis of (R)—N-((3-hydroxyphenyl)(4-isopropylphenyl)methyl)cyclopropanecarboxamide (Compound 62)

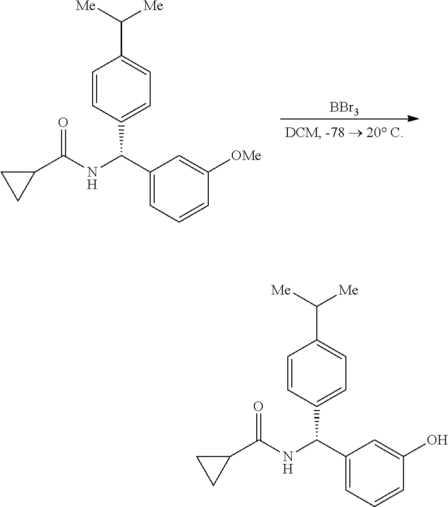 [SMILES: COc1cccc([C@H](NC(=O)C2CC2)c2ccc(C(C)C)cc2)c1 ; "BBr3" ; CC(C)c1ccc([C@@H](NC(=O)C2CC2)c2cccc(O)c2)cc1]

Step a: To a solution of (R)—N-((4-isopropylphenyl)(3-methoxyphenyl)methyl)cyclopropanecarboxamide (0.35 g, 1.08 mmol, 1 eq) in DCM (2 mL) at −78° C. was added BBr₃ (1.36 g, 5.41 mmol, 521 μL, 5 eq). The resulting mixture was warmed to 20° C. and stirred for 12 h. The reaction mixture was then diluted with saturated aqueous NaHCO₃ to adjust the solution pH to 7. The resulting biphasic mixture was then extracted with dichloromethane (2×50 mL). The combined organic extracts were washed with brine (30 mL), dried over anhydrous Na₂SO₄, filtered, and concentrated under reduced pressure. The crude residue obtained was purified by prep-HPLC to give (R)—N-((3-hydroxyphenyl)(4-isopropylphenyl)methyl)cyclopropanecarboxamide. LC-MS (ESI): m/z: [M+H]⁺ calculated for C₂₀H₂₃NO₂: 310.2; found 310.1.

### Example S-6: Synthesis of (1R,2S)-2-(2-acetamidoacetamido)-N—((S)-(4-isopropylphenyl(phenyl methyl cyclopentane-1-carboxamide (Compound 21)

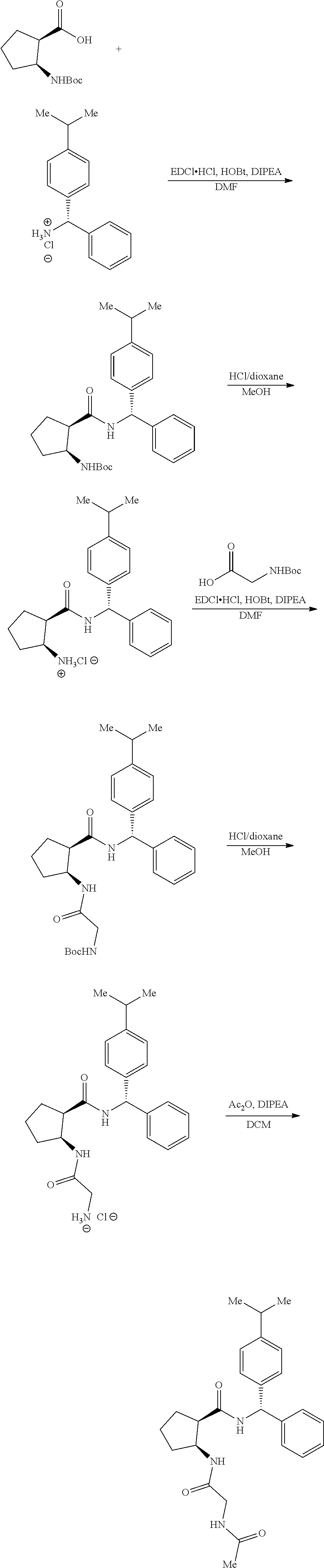

Step a: To a solution of (S)-(4-isopropylphenyl)(phenyl)methanaminium chloride (0.25 g, 0.95 mmol, 1 eq) and (1R,2S)-2-((tert-butoxycarbonyl)amino)cyclopentane-1-carboxylic acid (0.44 g, 1.91 mmol, 1 eq) in DMF (4.8 mL) was added EDCI·HCl (0.28 g, 1.4 mmol, 1.5 eq), 1-hydroxybenzotriazole hydrate (0.22 g, 1.43 mmol, 1.5 eq), and N,N-diisopropylethylamine (0.5 mL, 2.86 mmol, 3 eq). The resulting mixture was stirred at 25° C. for 4 h. The reaction mixture was then poured into water (150 mL), and the resulting solution was extracted with EtOAc (3×150 mL). The combined organic extracts were washed with brine, dried over anhydrous magnesium sulfate, filtered, and concentrated under reduced pressure. The crude residue obtained was purified by column chromatography to give tert-butyl ((1S,2R)-2-(((S)-(4-isopropylphenyl)(phenyl)methyl)carbamoyl)cyclopentyl)carbamate.

Step b: To a solution of tert-butyl ((1S,2R)-2-(((S)-(4-isopropylphenyl)(phenyl)methyl)carbamoyl)cyclopentyl)carbamate (0.4 g, 0.92 mmol, 1 eq) in methanol (4.6 mL) was added hydrochloric acid (1.14 mL, 4 M in dioxane, 4.6 mmol). The resulting reaction mixture was stirred at 25° C. for 2 h before the reaction mixture was concentrated under reduced pressure to give (1R,2S)-2-amino-N—((S)-(4-isopropylphenyl)(phenyl)methyl)cyclopentane-1-carboxamide hydrochloride salt.

Step c: To a solution of (1R,2S)-2-amino-N—((S)-(4-isopropylphenyl)(phenyl)methyl)cyclopentane-1-carboxamide hydrochloride salt (0.065 g, 0.17 mmol, 1 eq) and (tert-butoxycarbonyl)glycine (0.037 g, 0.209 mmol, 1.2 eq) in DMF (1 mL) was added EDCI·HCl (0.05 g, 0.261 mmol, 1.5 eq), 1-hydroxybenzotriazole hydrate (0.04 g, 0.261 mmol, 1.5 eq), and N,N-diisopropylethylamine (0.091 mL, 0.523 mmol, 3 eq). The resulting mixture was stirred at 25° C. for 16 h before the reaction mixture was diluted with EtOAc (15 mL) and H₂O (15 mL). The organic layer was collected and washed with H₂O (15 mL) and brine (15 mL), sequentially. The washed organic layer was dried over anhydrous magnesium sulfate, filtered, and concentrated under reduced pressure. The crude residue obtained was purified by prep-HPLC to give tert-butyl (2-(((1S,2R)-2-(((S)-(4-isopropylphenyl)(phenyl)methyl)carbamoyl)cyclopentyl)amino)-2-oxoethyl)carbamate.

Step d: To a solution of tert-butyl (2-(((1S,2R)-2-(((S)-(4-isopropylphenyl)(phenyl)methyl)carbamoyl)cyclopentyl)amino)-2-oxoethyl)carbamate (0.009 g, 0.018 mmol, 1 eq) in methanol (1 mL) was added hydrochloric acid (0.5 mL, 4 M in dioxane, 2 mmol, 18 eq). The resulting mixture was stirred at 25° C. for 4 h before the reaction mixture was concentrated under reduced pressure to give 2-(((1S,2R)-2-(((S)-(4-isopropylphenyl)(phenyl)methyl)carbamoyl)cyclopentyl)amino)-2-oxoethan-1-aminium chloride.

Step e: To a solution of 2-(((1S,2R)-2-(((S)-(4-isopropylphenyl)(phenyl)methyl)carbamoyl)cyclopentyl)amino)-2-oxoethan-1-aminium chloride (0.008 g, 0.019 mmol, 1 eq) in DCM (0.5 mL) was added N,N-diisopropylethylamine (0.007 mL, 0.038 mmol, 2 eq) and acetic anhydride (0.004 mL, 0.038 mmol, 2 eq), sequentially. The resulting mixture was stirred at 25° C. for 30 minutes. The reaction mixture was then concentrated under reduced pressure, and the crude residue obtained was purified by prep-HPLC to give (1R,2S)-2-(2-acetamidoacetamido)-N—((S)-(4-isopropylphenyl)(phenyl)methyl)cyclopentane-1-carboxamide. LC-MS (ESI): m/z: [M+H]⁺ calculated for C₂₆H₃₃N₃O₃: 436.3; found 436.3.

### Example S-7: Synthesis of (1R,2S)—N′—((R)-(4-isopropylphenyl)(o-tolyl)methyl)cyclopentane-1,2-dicarboxamide (Compound 30) and (1R,2S)-2-cyano-N—((R)-(4-isopropylphenyl)(o-tolyl)methyl)cyclopentane-1-carboxamide (Compound 7)

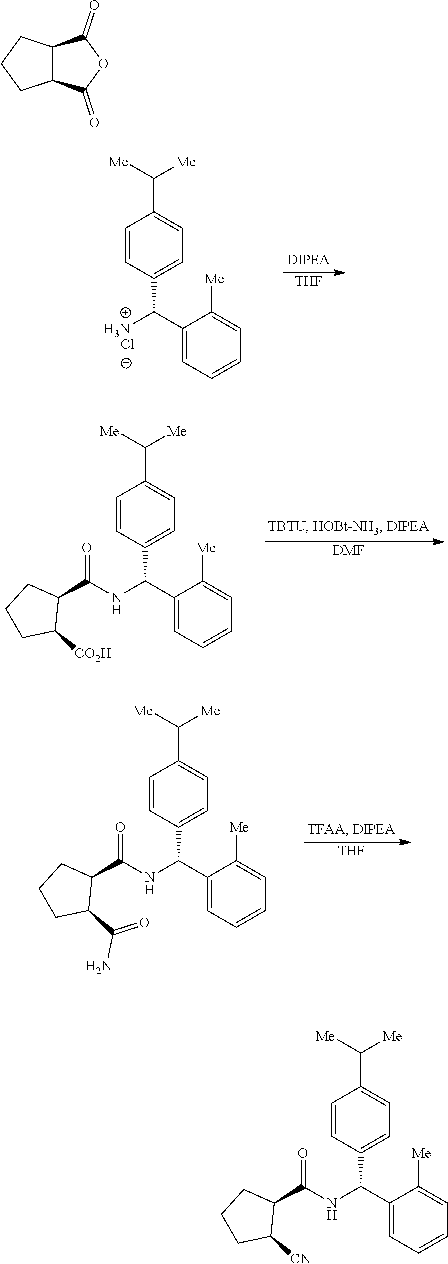

Step a: In two parallel reactions, (R)-(4-isopropylphenyl)(o-tolyl)methanaminium chloride (100 mg, 362 μmol, 1 eq) was dissolved in dry THE (3 mL). The resulting solution was cooled to 0° C. before N,N-diisopropylethylamine (93.7 mg, 725 μmol, 2 eq). The resulting mixture was stirred at 0° C. for 5 min before rel-(3aR,6aS)-tetrahydro-1H-cyclopenta[c]furan-1,3(3aH)-dione (76.2 mg, 543 umol, 1.5 eq) was added in one portion. The resulting reaction mixture was warmed to 25° C. and stirred for 5 h. The two reactions were then combined for work up. The combined reactions were cooled to 0° C. and quenched by addition of H₂O (10 mL). The pH of the resulting biphasic mixture was adjusted to pH=5 using 2 N aqueous HCl, and the resulting biphasic mixture was then extracted with EtOAc (3×20 mL). The combined organic extracts were washed with brine (10 mL), dried over anhydrous Na₂SO₄, filtered, and concentrated under reduced pressure. The crude residue obtained was purified by chiral SFC (column: DAICEL CHIRALPAK AD) to give (1S,2R)-2-(((R)-(4-isopropylphenyl)(o-tolyl)methyl)carbamoyl)cyclopentane-1-carboxylic acid as the second eluting isomer. LC-MS (ESI): m/z: [M+H]⁺ calculated for C₂₄H₂₉NO₃: 380.2; found 380.3.

Step b: To a solution of (1S,2R)-2-(((R)-(4-isopropylphenyl)(o-tolyl)methyl)carbamoyl)cyclopentane-1-carboxylic acid (81.5 mg, 0.21 mmol, 1 eq) in DMF (1 mL, 0.2 M) was added N,N-diisopropylethylamine (113 μL, 0.644 mmol, 3 eq), HOBt-NH₃ (390 mg, 0.26 mmol, 1.2 eq) and TBTU (70 mg, 0.21 mmol, 1 eq), and the resulting reaction mixture was stirred at 25° C. for 16 h. The reaction mixture was then diluted with EtOAc (30 mL) and H₂O (30 mL). The organic layer was collected and then washed with H₂O (30 mL) and brine (30 mL), sequentially. The resulting organic solution was dried over MgSO₄, filtered, and concentrated under reduced pressure. The resulting residue was purified by prep HPLC to give (1R,2S)—N¹—((R)-(4-isopropylphenyl)(o-tolyl)methyl)cyclopentane-1,2-dicarboxamide. LC-MS (ESI): m/z: [M+H]⁺ calculated for C₂₄H₃₀N₂O₂: 379.2; found 379.2.

Step c: (1R,2S)—N¹—((R)-(4-isopropylphenyl)(o-tolyl)methyl)cyclopentane-1,2-dicarboxamide (48 mg, 0.127 mmol, 1 eq) in dichloromethane (1 mL) was added TFAA (0.027 mL, 0.19 mmol, 1.5 eq), and N,N-diisopropylethylamine (0.044 mL, 0.254 mmol, 2 eq). The reaction mixture was stirred at 25° C. for 4 h before it was concentrated under reduced pressure and purified by column chromatography to give (1R,2S)-2-cyano-N—((R)-(4-isopropylphenyl)(o-tolyl)methyl)cyclopentane-1-carboxamide. LC-MS (ESI): m/z: [M+H]⁺ calculated for C₂₄H₂₈N₂O; 361.2; found 361.3.

### Example S-8: Synthesis of (1S,2R)—N¹-cyano-N²—((R)-(4-isopropylphenyl)(o-tolyl)methyl)cyclopentane-1,2-dicarboxamide (Compound 22)

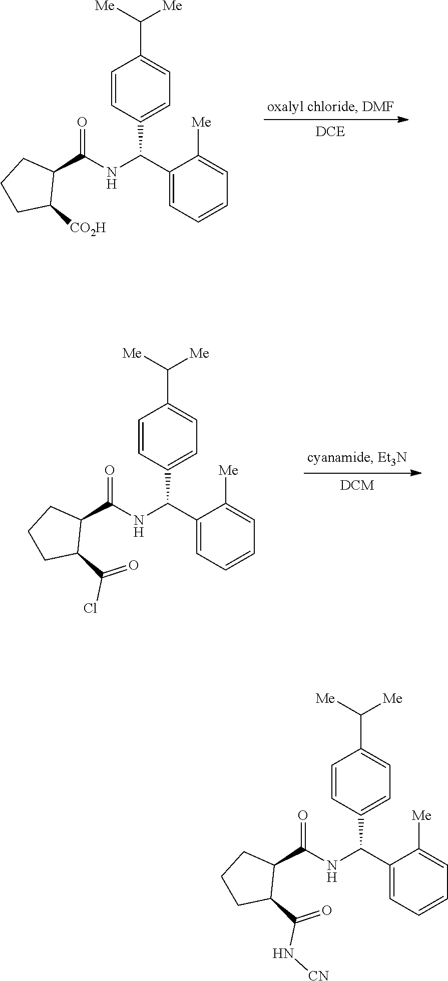 [SMILES: Cc1ccccc1[C@H](NC(=O)[C@@H]1CCC[C@@H]1C(=O)O)c1ccc(C(C)C)cc1 ; Cc1ccccc1[C@H](NC(=O)[C@@H]1CCC[C@@H]1C(=O)Cl)c1ccc(C(C)C)cc1 ; Cc1ccccc1[C@H](NC(=O)[C@@H]1CCC[C@@H]1C(=O)NC#N)c1ccc(C(C)C)cc1]

Step a: To a solution of (1S,2R)-2-(((R)-(4-isopropylphenyl)(o-tolyl)methyl)carbamoyl)cyclopentane-1-carboxylic acid (25 mg, 0.066 mmol, 1 eq) in DCE (1 mL) was added oxalyl chloride (0.05 mL, 2 M, 0.099 mmol, 1.5 eq) followed by one drop of DMF. The resulting mixture was stirred at 25° C. for 16 h before the reaction mixture was concentrated under reduced pressure to give crude (1S,2R)-2-(((R)-(4-isopropylphenyl)(o-tolyl)methyl)carbamoyl)cyclopentane-1-carbonyl chloride, which was directly used in the next step without further purification or characterization.

Step b: To a solution of cyanamide (5 mg, 0.131 mmol, 2 eq) and triethylamine (28*L, 0.196 mmol, 3 eq) in DCM (1 mL) under N₂ atmosphere at 25° C. was added a solution of (1S,2R)-2-(((R)-(4-isopropylphenyl)(o-tolyl)methyl)carbamoyl)cyclopentane-1-carbonyl chloride (26 mg, 0.065 mmol, 1 eq) in DCM (1 mL) in a dropwise manner. The resulting mixture was stirred for 16 h at 25° C. under N₂ atmosphere. The reaction mixture was then concentrated under reduced pressure and purified by prep-HPLC to give (1S,2R)—N¹-cyano-N²—((R)-(4-isopropylphenyl)(o-tolyl)methyl)cyclopentane-1,2-dicarboxamide. LC-MS (ESI): m/z: [M+H]⁺ calculated for C₂₅H₂₉N₃O₂: 404.2; found 404.3.

### Example S-9: Synthesis of (1R,2S)—N¹—((R)-(4-isopropylphenyl)(o-tolyl)methyl)-N²-methylcyclopentane-1,2-dicarboxamide (Compound 16)

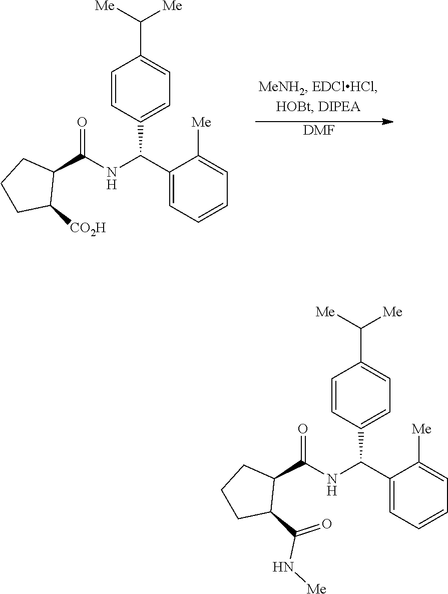

Step a: To a solution of (1S,2R)-2-(((R)-(4-isopropylphenyl)(o-tolyl)methyl)carbamoyl)cyclopentane-1-carboxylic acid (50 mg, 0.132 mmol, 1 eq) in DMF (0.5 mL) was added EDCI·HCl (38 mg, 0.198 mmol, 1.5 eq), 1-hydroxybenzotriazole hydrate (0.03 g, 0.198 mmol, 1.5 eq), and N,N-diisopropylethylamine (0.069 mL, 0.395 mmol, 3 eq). The resulting mixture was stirred at 25° C. for 16 h before the reaction mixture was diluted with EtOAc (20 mL) and water (20 mL). The organic layer was collected and then washed with water (20 mL) and brine (20 mL), sequentially. The organic solution was then dried over anhydrous magnesium sulfate, filtered, and concentrated under reduced pressure. The crude residue was purified by prep-HPLC to give (1R,2S)—N¹—((R)-(4-isopropylphenyl)(o-tolyl)methyl)-N²-methylcyclopentane-1,2-dicarboxamide. LC-MS (ESI): m/z: [M+H]⁺ calculated for C₂₅H₃₂N₂O₂; 393.2 found 393.2.

The following compounds in Table T-1 were synthesized using procedures similar to Examples S-1 through S-9 using the appropriate starting materials and reagents.

TABLE T-1

| | | | Exact | LCMS, |
|---|---|---|---|---|
| Cmpd. | | | mass | Found |
| No. | Structure | IUPAC | (g/mol) | \|[M + H]⁺ |
| 1 | 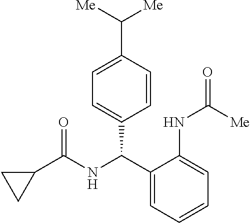 [SMILES: CC(=O)Nc1ccccc1[C@H](NC(=O)C1CC1)c1ccc(C(C)C)cc1] | (R)-N-((2- acetamidophenyl)(4- isopropylphenyl)methyl) cyclopropanecarboxamide | 350.2 | 351.2 |
| 2 | 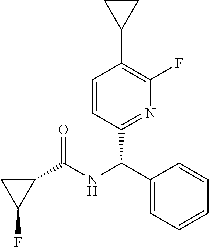 [SMILES: O=C(N[C@@H](c1ccccc1)c1ccc(C2CC2)c(F)n1)[C@H]1C[C@@H]1F] | (1R,2S)-N-((S)-(5- cyclopropyl-6- fluoropyridin-2- yl)(phenyl)methyl)-2- fluorocyclopropane-1- carboxamide | 328.1 | 329.2 |
| 4 | 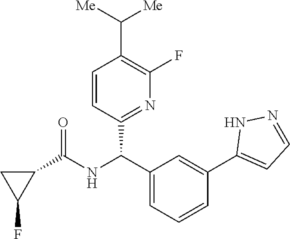 [SMILES: CC(C)c1ccc([C@@H](NC(=O)[C@H]2C[C@@H]2F)c2cccc(-c3ccn[nH]3)c2)nc1F] | (1R,2S)-N-((S)-(3-(1H- pyrazol-5-yl)phenyl)(6- fluoro-5-isopropylpyridin- 2-yl)methyl)-2- fluorocyclopropane-1- carboxamide (SFC column: REGIS (s,s) WHELK-O1, first eluting isomer) | 396.2 | 397.1 |
| 5 | 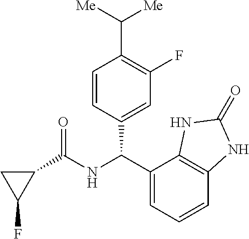 [SMILES: CC(C)c1ccc([C@@H](NC(=O)[C@H]2C[C@@H]2F)c2cccc3[nH]c(=O)[nH]c23)cc1F] | (1R,2S)-2-fluoro-N- ((R)-(3-fluoro-4- isopropylphenyl)(2-oxo- 2,3-dihydro-1H- benzo[d]imidazol-4- yl)methyl)cyclopropane-1- carboxamide (SFC column: DAICEL CHIRALPAK IC, first eluting isomer) | 385.2 | 386.0 |
| 6 | 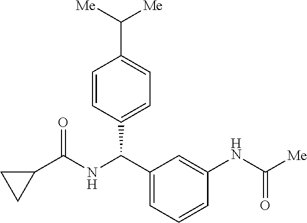 [SMILES: CC(=O)Nc1cccc([C@H](NC(=O)C2CC2)c2ccc(C(C)C)cc2)c1] | (R)-N-((3- acetamidophenyl)(4- isopropylphenyl)methyl) cyclopropanecarboxamide | 350.2 | 351.2 |
| 8 | 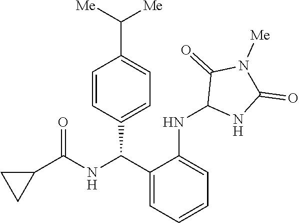 [SMILES: CC(C)c1ccc([C@@H](NC(=O)C2CC2)c2ccccc2NC2NC(=O)N(C)C2=O)cc1] | N-((1R)-(4- isopropylphenyl)(2-((1- methyl-2,5- dioxoimidazolidin-4- yl)amino)phenyl)methyl) cyclopropanecarboxamide | 420.2 | 419.1 (M − H) |
| 9 | 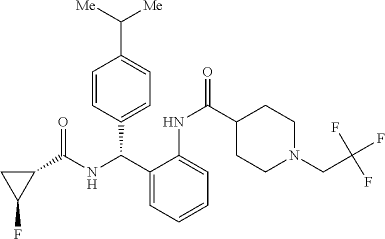 [SMILES: CC(C)c1ccc([C@@H](NC(=O)[C@H]2C[C@@H]2F)c2ccccc2NC(=O)C2CCN(CC(F)(F)F)CC2)cc1] | N-(2-((R)-((1R,2S)-2- fluorocyclopropane-1- carboxamido)(4- isopropylphenyl)methyl) phenyl)-1-(2,2,2- trifluoroethyl)piperidine-4- carboxamide | 519.3 | 520.3 |
| 10 | 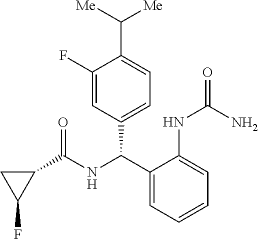 [SMILES: CC(C)c1ccc([C@@H](NC(=O)[C@H]2C[C@@H]2F)c2ccccc2NC(N)=O)cc1F] | (1R,2S)-2-fluoro-N- ((R)-(3-fluoro-4- isopropylphenyl)(2- ureidophenyl)methyl) cyclopropane- 1-carboxamide (SFC column: DAICEL CHIRALPAK IC, second eluting isomer) | 387.2 | 388.1 |
| 11 | 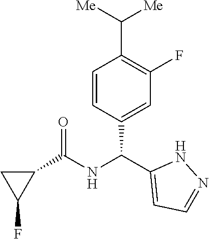 [SMILES: CC(C)c1ccc([C@@H](NC(=O)[C@H]2C[C@@H]2F)c2ccn[nH]2)cc1F] | (1R,2S)-2-fluoro-N- ((R)-(3-fluoro-4- isopropylphenyl)(1H- pyrazol-5- yl)methyl)cyclopropane-1- carboxamide (SFC column: DAICEL CHIRALPAK IC, first eluting isomer) | 319.1 | 320.1 |
| 12 | 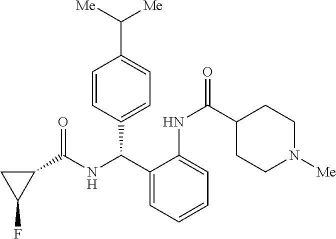 [SMILES: CC(C)c1ccc([C@@H](NC(=O)[C@H]2C[C@@H]2F)c2ccccc2NC(=O)C2CCN(C)CC2)cc1] | N-(2-((R)-((1R,2S)-2- fluorocyclopropane-1- carboxamido)(4- isopropylphenyl)methyl) phenyl)-1- methylpiperidine- 4-carboxamide | 451.3 | 452.2 |
| 13 | 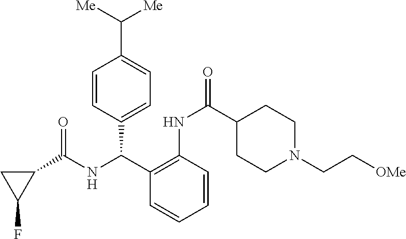 [SMILES: COCCN1CCC(C(=O)Nc2ccccc2[C@H](NC(=O)[C@H]2C[C@@H]2F)c2ccc(C(C)C)cc2)CC1] | N-(2-((R)-((1R,2S)-2- fluorocyclopropane-1- carboxamido)(4- isopropylphenyl)methyl) phenyl)-1-(2- methoxyethyl)piperidine-4- carboxamide | 495.3 | 496.3 |
| 14 | 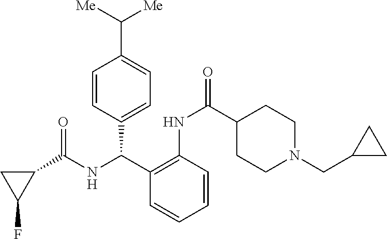 [SMILES: CC(C)c1ccc([C@@H](NC(=O)[C@H]2C[C@@H]2F)c2ccccc2NC(=O)C2CCN(CC3CC3)CC2)cc1] | 1-(cyclopropylmethyl)-N- (2-((R)-((1R,2S)-2- fluorocyclopropane-1- carboxamido)(4- isopropylphenyl)methyl) phenyl)piperidine-4- carboxamide | 491.3 | 492.3 |
| 15 | 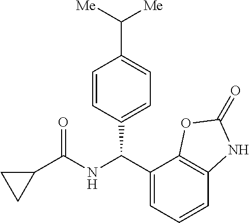 [SMILES: CC(C)c1ccc([C@@H](NC(=O)C2CC2)c2cccc3[nH]c(=O)oc23)cc1] | (R)-N-((4- isopropylphenyl)(2-oxo- 2,3- dihydrobenzo[d]oxazol-7- yl)methyl)cyclo- propanecarboxamide (SFC column: DAICEL CHIRALPAK AD, second eluting isomer) | 350.2 | 351.1 |
| 17 | 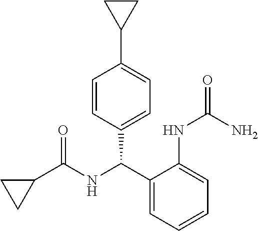 [SMILES: NC(=O)Nc1ccccc1[C@H](NC(=O)C1CC1)c1ccc(C2CC2)cc1] | (R)-N-((4- cyclopropylphenyl)(2- ureidophenyl)methyl) cyclopropane- carboxamide (SFC column: DAICEL CHIRALPAK AD, first eluting isomer) | 349.2 | 350.2 |
| 18 | 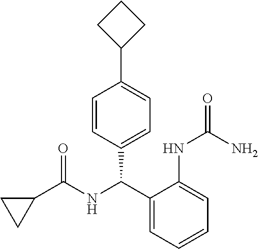 [SMILES: NC(=O)Nc1ccccc1[C@H](NC(=O)C1CC1)c1ccc(C2CCC2)cc1] | (R)-N-((4- cyclobutylphenyl)(2- ureidophenyl)methyl) cyclopropane- carboxamide (SFC column: DAICEL CHIRALPAK IC, first eluting isomer) | 363.2 | 364.1 |
| 19 | 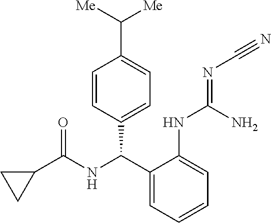 [SMILES: CC(C)c1ccc([C@@H](NC(=O)C2CC2)c2ccccc2N/C(N)=N/C#N)cc1] | (R,E)-N-((2-(2- cyanoguanidino)phenyl)(4- isopropylphenyl)methyl) cyclopropanecarboxamide | 375.2 | 376.2 |
| 20 | 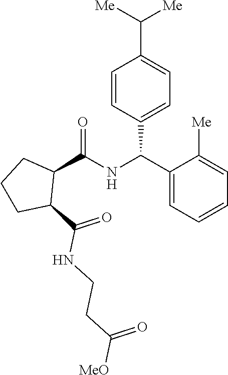 [SMILES: COC(=O)CCNC(=O)[C@H]1CCC[C@H]1C(=O)N[C@H](c1ccc(C(C)C)cc1)c1ccccc1C] | methyl 3-((1S,2R)-2-(((R)- (4-isopropylphenyl)(o- tolyl)methyl)carbamoyl) cyclopentane-1- carboxamido)propanoate | 464.3 | 465.2 |
| 23 | 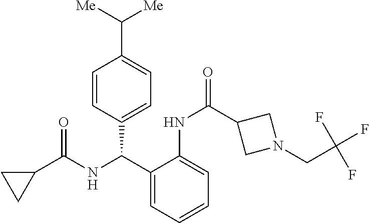 [SMILES: CC(C)c1ccc([C@@H](NC(=O)C2CC2)c2ccccc2NC(=O)C2CN(CC(F)(F)F)C2)cc1] | (R)-N-(2- (cyclopropane- carboxamido(4- isopropylphenyl)methyl) phenyl)-1-(2,2,2- trifluoroethyl)azetidine-3- carboxamide | 473.2 | 474.1 |
| 24 | 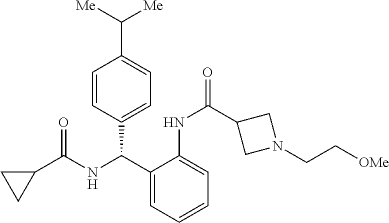 [SMILES: COCCN1CC(C(=O)Nc2ccccc2[C@H](NC(=O)C2CC2)c2ccc(C(C)C)cc2)C1] | (R)-N-(2- (cyclopropane- carboxamido(4- isopropylphenyl)methyl) phenyl)-1-(2- methoxyethyl)azetidine-3- carboxamide | 449.3 | 450.1 |
| 25 | 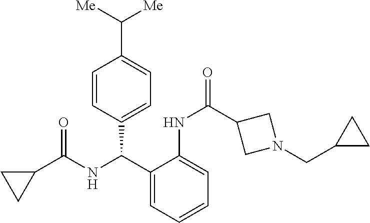 [SMILES: CC(C)c1ccc([C@@H](NC(=O)C2CC2)c2ccccc2NC(=O)C2CN(CC3CC3)C2)cc1] | (R)-N-(2- (cyclopropane- carboxamido(4- isopropylphenyl)methyl) phenyl)-1- (cyclopropylmethyl) azetidine-3-carboxamide | 445.3 | 446.3 |
| 27 | 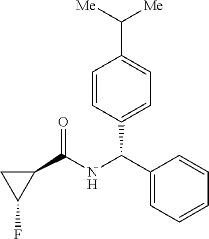 [SMILES: CC(C)c1ccc([C@@H](NC(=O)[C@@H]2C[C@H]2F)c2ccccc2)cc1] | (1S,2R)-2-fluoro-N-((S)-(4- isopropylphenyl)(phenyl) methyl)cyclopropane-1- carboxamide | 311.2 | 312.1 |
| 28 | 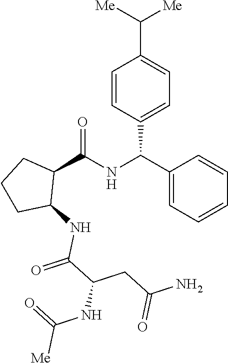 [SMILES: CC(=O)N[C@@H](CC(N)=O)C(=O)N[C@H]1CCC[C@H]1C(=O)N[C@@H](c1ccccc1)c1ccc(C(C)C)cc1] | (S)-2-acetamido-N¹- ((1S,2R)-2-(((S)-(4- isopropylphenyl)(phenyl) methyl)carbamoyl) cyclopentyl)succinamide | 492.3 | 493.2 |
| 29 | 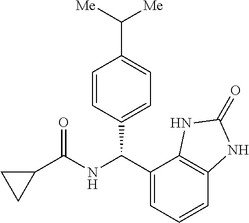 [SMILES: CC(C)c1ccc([C@@H](NC(=O)C2CC2)c2cccc3[nH]c(=O)[nH]c23)cc1] | (R)-N-((4- isopropylphenyl)(2-oxo- 2,3-dihydro-1H- benzo[d]imidazol-4- yl)methyl)cyclo- propanecarboxamide (SFC column: REGIS (s,s) WHELK-O1, first eluting isomer) | 349.2 | 350.1 |
| 32 | 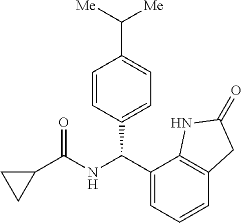 [SMILES: CC(C)c1ccc([C@@H](NC(=O)C2CC2)c2cccc3c2NC(=O)C3)cc1] | (R or S)-N-((4- isopropylphenyl)(2- oxoindolin-7- yl)methyl)cyclo- propanecarboxamide (SFC column: REGIS (s,s) WHELK-O1, first eluting isomer) | 348.2 | 349.2 |
| 33 | 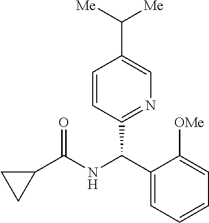 [SMILES: COc1ccccc1[C@H](NC(=O)C1CC1)c1ccc(C(C)C)cn1] | (S)-N-((5-isopropylpyridin- 2-yl)(2- methoxyphenyl)methyl) cyclopropanecarboxamide (SFC column: DAICEL CHIRALPAK AD, second eluting isomer) | 324.2 | 325.2 |
| 34 | 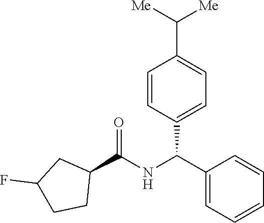 [SMILES: CC(C)c1ccc([C@@H](NC(=O)[C@H]2CCC(F)C2)c2ccccc2)cc1] | (1S)-3-fluoro-N-((S)-(4- isopropylphenyl)(phenyl) methyl)cyclopentane-1- carboxamide | 339.2 | 340.1 |
| 35 | 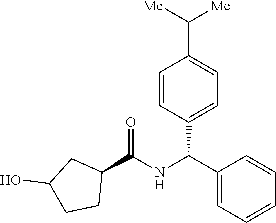 [SMILES: CC(C)c1ccc([C@@H](NC(=O)[C@H]2CCC(O)C2)c2ccccc2)cc1] | (1S)-3-hydroxy-N-((S)-(4- isopropylphenyl)(phenyl) methyl)cyclopentane-1- carboxamide | 337.2 | 338.1 |
| 36 | 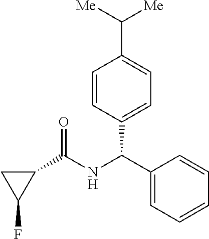 [SMILES: CC(C)c1ccc([C@@H](NC(=O)[C@H]2C[C@@H]2F)c2ccccc2)cc1] | (1R,2S)-2-fluoro-N-((S)-(4- isopropylphenyl)(phenyl) methyl)cyclopropane-1- carboxamide | 311.2 | 312.1 |
| 38 | 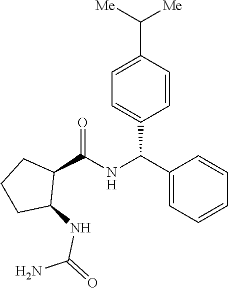 [SMILES: CC(C)c1ccc([C@@H](NC(=O)[C@@H]2CCC[C@@H]2NC(N)=O)c2ccccc2)cc1] | (1R,2S)-N-((S)-(4- isopropylphenyl)(phenyl) methyl)-2- ureidocyclopentane-1- carboxamide | 379.2 | 380.3 |
| 39 | 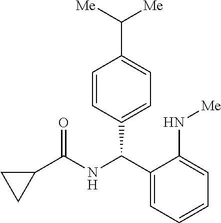 [SMILES: CNc1ccccc1[C@H](NC(=O)C1CC1)c1ccc(C(C)C)cc1] | (R)-N-((4- isopropylphenyl)(2- (methylamino)phenyl) methyl)cyclo- propanecarboxamide (SFC column: REGIS (s,s) WHELK-O1, first eluting isomer) | 322.2 | 323.2 |
| 40 | 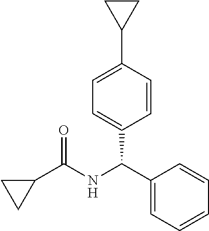 [SMILES: O=C(N[C@@H](c1ccccc1)c1ccc(C2CC2)cc1)C1CC1] | (S)-N-((4- cyclobutylphenyl)(phenyl) methyl)cyclo- propanecarboxamide | 305.2 | 306.1 |
| 41 | 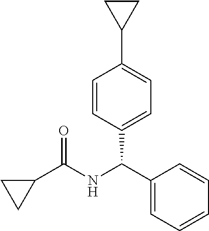 [SMILES: O=C(N[C@@H](c1ccccc1)c1ccc(C2CC2)cc1)C1CC1] | (S)-N-((4- cyclopropylphenyl) (phenyl)methyl) cyclopropanecarboxamide | 291.2 | 292.1 |
| 42 | 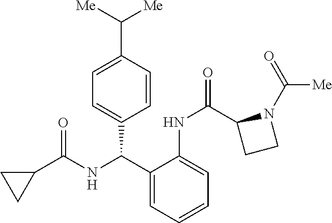 [SMILES: CC(=O)N1CC[C@H]1C(=O)Nc1ccccc1[C@H](NC(=O)C1CC1)c1ccc(C(C)C)cc1] | (S)-1-acetyl-N-(2-((R)- cyclopropanecarboxamido (4- isopropylphenyl)methyl) phenyl)azetidine-2- carboxamide | 433.2 | 434.3 |
| 43 | 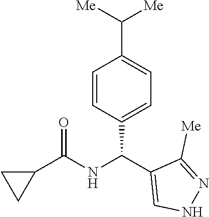 [SMILES: Cc1n[nH]cc1[C@H](NC(=O)C1CC1)c1ccc(C(C)C)cc1] | (R)-N-((4- isopropylphenyl)(3- methyl-1H-pyrazol-4- yl)methyl)cyclo- propanecarboxamide (SFC column: REGIS (s,s) WHELK-O1, second eluting isomer) | 297.2 | 298.2 |
| 44 | 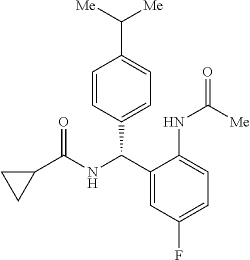 [SMILES: CC(=O)Nc1ccc(F)cc1[C@H](NC(=O)C1CC1)c1ccc(C(C)C)cc1] | (R)-N-((2-acetamido-5- fluorophenyl)(4- isopropylphenyl)methyl) cyclopropanecarboxamide (SFC column: DAICEL CHIRALPAK AD, first eluting isomer) | 368.2 | 369.1 |
| 45 | 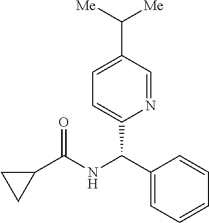 [SMILES: CC(C)c1ccc([C@@H](NC(=O)C2CC2)c2ccccc2)nc1] | (S)-N-((5-isopropyl- pyridin-2- yl)(phenyl)methyl) cyclopropanecarboxamide | 294.2 | 294.9 |
| 46 | 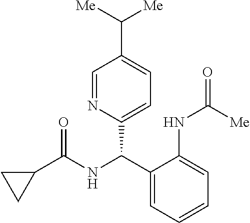 [SMILES: CC(=O)Nc1ccccc1[C@H](NC(=O)C1CC1)c1ccc(C(C)C)cn1] | (S)-N-((2- acetamidophenyl)(5- isopropylpyridin-2- yl)methyl) cyclopropanecarboxamide | 351.2 | 352.1 |
| 47 | 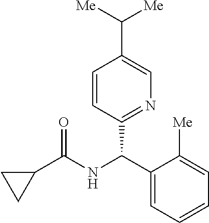 [SMILES: Cc1ccccc1[C@H](NC(=O)C1CC1)c1ccc(C(C)C)cn1] | (S)-N-((5-isopropylpyridin- 2-yl)(o- tolyl)methyl)cyclopropane- carboxamide (SFC column: DAICEL CHIRALPAK AD, second eluting isomer) | 308.2 | 309.2 |
| 49 | 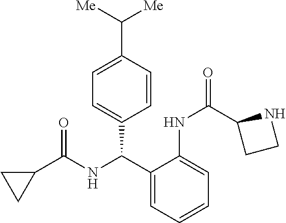 [SMILES: CC(C)c1ccc([C@@H](NC(=O)C2CC2)c2ccccc2NC(=O)[C@@H]2CCN2)cc1] | (S)-N-(2-((R)- cyclopropanecarboxamido (4- isopropylphenyl)methyl) phenyl)azetidine-2- carboxamide | 391.2 | 392.4 |
| 50 | 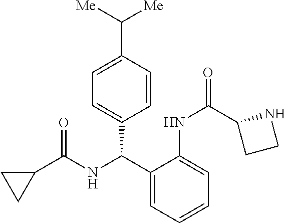 [SMILES: CC(C)c1ccc([C@@H](NC(=O)C2CC2)c2ccccc2NC(=O)[C@H]2CCN2)cc1] | (R)-N-(2-((R)- cyclopropanecarboxamido (4- isopropylphenyl)methyl) phenyl)azetidine-2- carboxamide | 391.2 | 392.4 |
| 51 | 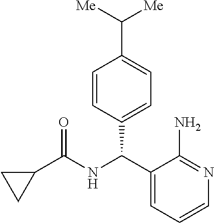 [SMILES: CC(C)c1ccc([C@@H](NC(=O)C2CC2)c2cccnc2N)cc1] | (R)-N-((2-aminopyridin-3- yl)(4- isopropylphenyl)methyl) cyclopropanecarboxamide (SFC column: DAICEL CHIRALPAK AD, first eluting isomer) | 309.2 | 310.2 |
| 52 | 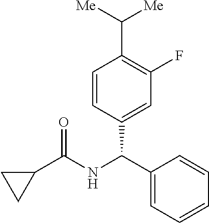 [SMILES: CC(C)c1ccc([C@@H](NC(=O)C2CC2)c2ccccc2)cc1F] | (S)-N-((3-fluoro-4- isopropylphenyl)(phenyl) methyl)cyclo- propanecarboxamid (SFC column: DAICEL CHIRALPAK AD, second eluting isomer) | 311.2 | 312.2 |
| 53 | 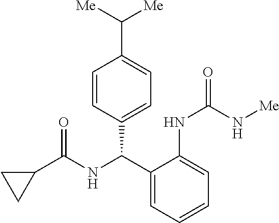 [SMILES: CNC(=O)Nc1ccccc1[C@H](NC(=O)C1CC1)c1ccc(C(C)C)cc1] | (R)-N-((4- isopropylphenyl)(2-(3- methylureido)phenyl) methyl)cyclo- propanecarboxamide | 365.2 | 366.2 |
| 54 | 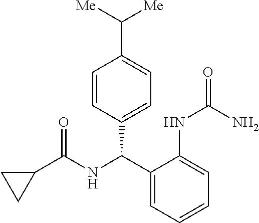 [SMILES: CC(C)c1ccc([C@@H](NC(=O)C2CC2)c2ccccc2NC(N)=O)cc1] | (R)-N-((4- isopropylphenyl)(2- ureidophenyl)methyl) cyclopropanecarboxamide | 351.2 | 352.2 |
| 55 | 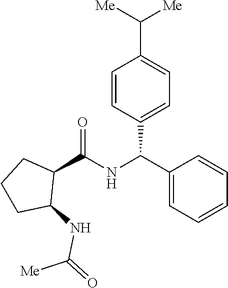 [SMILES: CC(=O)N[C@H]1CCC[C@H]1C(=O)N[C@@H](c1ccccc1)c1ccc(C(C)C)cc1] | (1R,2S)-2-acetamido-N- ((S)-(4- isopropylphenyl)(phenyl) methyl)cyclopentane-1- carboxamide | 378.2 | 379.2 |
| 56 | 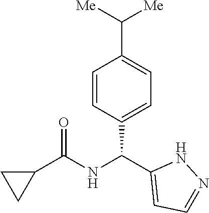 [SMILES: CC(C)c1ccc([C@@H](NC(=O)C2CC2)c2ccn[nH]2)cc1] | (R)-N-((4- isopropylphenyl)(1H- pyrazol-5-yl)methyl) cyclopropanecarboxamide | 283.2 | 284.2 |
| 57 | 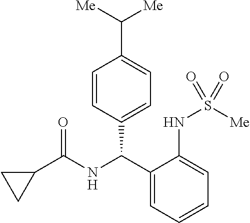 [SMILES: CC(C)c1ccc([C@@H](NC(=O)C2CC2)c2ccccc2NS(C)(=O)=O)cc1] | (R)-N-((4- isopropylphenyl)(2- (methylsulfonamido) phenyl)methyl) cyclopropanecarboxamide | 386.2 | 387.2 |
| 58 | 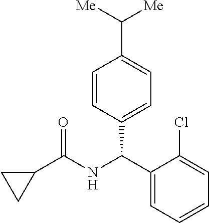 [SMILES: CC(C)c1ccc([C@@H](NC(=O)C2CC2)c2ccccc2Cl)cc1] | (R)-N-((2-chlorophenyl)(4- isopropylphenyl)methyl) cyclopropanecarboxamide (SFC column: REGIS (s,s) WHELK-O1, first eluting isomer) | 327.1 | 328.1 |
| 59 | 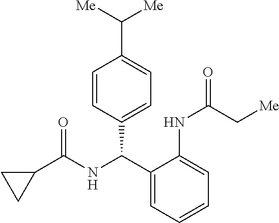 [SMILES: CCC(=O)Nc1ccccc1[C@H](NC(=O)C1CC1)c1ccc(C(C)C)cc1] | (R)-N-((4- isopropylphenyl)(2- propionamidophenyl) methyl)cyclo- propanecarboxamide | 364.2 | 365.4 |
| 60 | 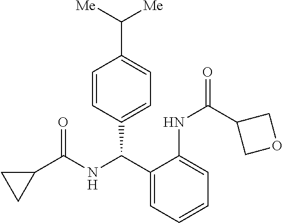 [SMILES: CC(C)c1ccc([C@@H](NC(=O)C2CC2)c2ccccc2NC(=O)C2COC2)cc1] | (R)-N-(2- (cyclopropanecarboxamido (4- isopropylphenyl)methyl) phenyl)oxetane-3- carboxamide | 392.2 | 393.3 |
| 61 | 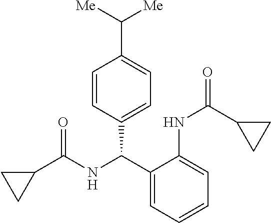 [SMILES: CC(C)c1ccc([C@@H](NC(=O)C2CC2)c2ccccc2NC(=O)C2CC2)cc1] | (R)-N-(2- (cyclopropanecarboxamido (4-isopropylphenyl) methyl)phenyl) cyclopropanecarboxamide (SFC column: DAICEL CHIRALPAK AD, first eluting isomer) | 376.2 | 377.2 |
| 63 | 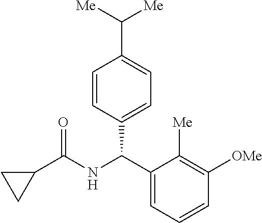 [SMILES: COc1cccc([C@H](NC(=O)C2CC2)c2ccc(C(C)C)cc2)c1C] | (R)-N-((4- isopropylphenyl)(3- methoxy-2- methylphenyl)methyl) cyclopropanecarboxamide | 337.2 | 338.2 |
| 65 | 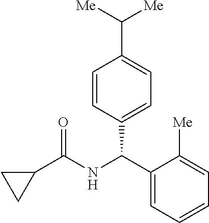 [SMILES: Cc1ccccc1[C@H](NC(=O)C1CC1)c1ccc(C(C)C)cc1] | (R)-N-((4- isopropylphenyl)(o- tolyl)methyl)cyclopropane- carboxamide (SFC column: Phenomenex- Cellulose-2, first eluting isomer) | 307.2 | 308.3 |
| 66 | 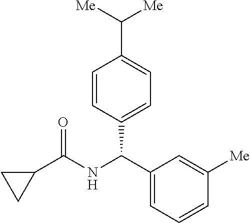 [SMILES: Cc1cccc([C@H](NC(=O)C2CC2)c2ccc(C(C)C)cc2)c1] | (R)-N-((4- isopropylphenyl)(m- tolyl)methyl)cyclopropane- carboxamide (SFC column: REGIS (s,s) WHELK-O1, first eluting isomer) | 307.2 | 308.2 |
| 67 | 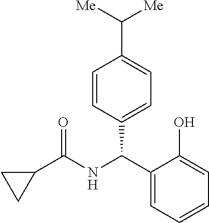 [SMILES: CC(C)c1ccc([C@@H](NC(=O)C2CC2)c2ccccc2O)cc1] | (R)-N-((2- hydroxyphenyl)(4- isopropylphenyl)methyl) cyclopropanecarboxamide | 309.2 | 310.1 |
| 68 | 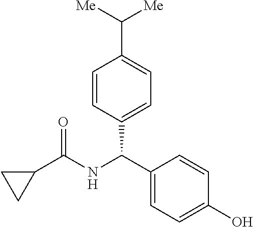 [SMILES: CC(C)c1ccc([C@@H](NC(=O)C2CC2)c2ccc(O)cc2)cc1] | (R)-N-((4- hydroxyphenyl)(4- isopropylphenyl)methyl) cyclopropanecarboxamide | 309.2 | 310.1 |
| 69 | 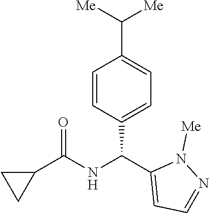 [SMILES: CC(C)c1ccc([C@@H](NC(=O)C2CC2)c2ccnn2C)cc1] | (R)-N-((4- isopropylphenyl)(1- methyl-1H-pyrazol-5- yl)methyl)cyclo- propanecarboxamide (prep-HPLC column: DAICEL CHIRALPAK AS, first eluting isomer) | 297.2 | 298.2 |
| 70 | 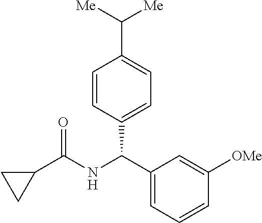 [SMILES: COc1cccc([C@H](NC(=O)C2CC2)c2ccc(C(C)C)cc2)c1] | (R)-N-((4- isopropylphenyl)(3- methoxyphenyl)methyl) cyclopropanecarboxamide | 323.2 | 324.1 |
| 71 | 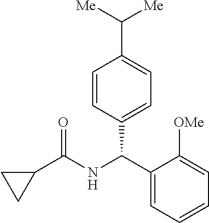 [SMILES: COc1ccccc1[C@H](NC(=O)C1CC1)c1ccc(C(C)C)cc1] | (R)-N-((4- isopropylphenyl)(2- methoxyphenyl)methyl) cyclopropanecarboxamide (SFC column: Chiralpak AD-3, second eluting isomer) | 323.2 | 324.2 |
| 72 | 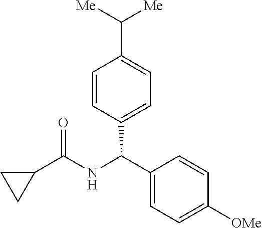 [SMILES: COc1ccc([C@H](NC(=O)C2CC2)c2ccc(C(C)C)cc2)cc1] | (R)-N-((4- isopropylphenyl)(4- methoxyphenyl)methyl) cyclopropanecarboxamide | 323.2 | 324.2 |
| 73 | 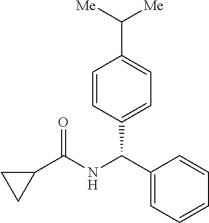 [SMILES: CC(C)c1ccc([C@@H](NC(=O)C2CC2)c2ccccc2)cc1] | (S)-N-((4- isopropylphenyl)(phenyl) methyl)cyclo- propanecarboxamide | 293.2 | 294.1 |

### BIOLOGICAL EXAMPLES

### Example B-1

The GYS1 coupled enzyme assay is a kinetic biochemical assay that indirectly quantifies the rate of glycogen synthesis by coupling the conversion of GYS1 substrate UDP-glucose into UDP with downstream enzymatic reactions. UDP is released from UDP-glucose as glucose monomers are linked into the growing glycogen strand by GYS1. The coupled assay then proceeds with pyruvate kinase utilizing UDP and phospho(enol)pyruvate (PEP) to form pyruvate. Lactate dehydrogenase then converts pyruvate and NADH into lactate and NAD+. Oxidation of NADH to NAD+ can be measured continuously with a plate reader by quantifying the decrease in NADH absorbance at 340 nm over time.

Compounds that inhibit the hGYS1 enzyme and, subsequently, the downstream conversion of NADH to NAD+, were tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP at 1 mM were added before splitting buffer into hGYS1 buffer and substrate buffer. To the hGYS1 buffer, rabbit liver glycogen was added at a final concentration of 0.2% glycogen. Glucose-6-Phosphate was added at 1 mM, recombinant hGYS1/GN1 protein was added at 50 nM to the substrate buffer, phosphoenolpyruvate (PEP) was added at 2 mM, UDP-Glucose was added at 0.8 mM, NADH) was added at 0.6 mM, and Pyruvate Kinase/Lactate Dehydrogenase was added at 20 units/mL. The reaction was initiated by mixing hGYS1 buffer and substrate buffer at a 1:1 ratio. Both buffers were plated using a liquid dispensing device with hGYS1 buffer plated first followed by the substrate buffer. Plates were spun briefly to eliminate air bubbles and are immediately read in continuous mode at an absorbance of 340 nm, for 10 time points in one-minute increments, for a total of 10 minutes. The slope from these 10 time points was normalized to the positive and negative control wells. The duplicate % inhibition values are then averaged and fit to a Hill equation for dose response according to the Levenberg-Marquardt algorithm with the Hill equation maximum set to 100 and the minimum set to 0.

The results are shown in Table 3 below, which reports the IC₅₀ of each compound. Unless otherwise specified, IC₅₀ values are reported as the geometric mean of at least 2 assay runs on separate days. Each run represents the average of a technical replicate, where each compound was assayed twice in the same plate. As shown in the table below, the compounds of the present invention are potent inhibitors of human GYS1.

Note that, in Table 3, the compounds are referred to by the corresponding Compound No. in Table 1, which is also referred to in the synthetic examples.

TABLE 3

| | GYS1 |
|---|---|
| | PK/LDH |
| Cmpd | IC₅₀ |
| No. | (μM) |
| 1 | 0.285 |
| 2 | 1.145 |
| 3 | 0.055 |
| 4 | 0.156 |
| 5 | 0.131 |
| 6 | 0.737 |
| 7 | 1.141 |
| 8 | 0.295 |
| 9 | 0.956 |
| 10 | 0.194 |
| 11 | 0.145 |
| 12 | 1.637 |
| 13 | 1.043 |
| 14 | 0.891 |
| 15 | 0.883 |
| 16 | 1.742 |
| 17 | 2.216 |
| 18 | 0.964 |
| 19 | 0.304 |
| 20 | 2.979 |
| 21 | 1.527 |
| 22 | 1.283 |
| 23 | 1.213 |
| 24 | 1.200 |
| 25 | 1.143 |
| 26 | 1.188 |
| 27 | 0.598 |
| 28 | 2.973 |
| 29 | 0.169 |
| 30 | 1.395 |
| 31 | 1.224 |
| 32 | 2.004 |
| 33 | 1.423 |
| 34 | 0.778 |
| 35 | 1.134 |
| 36 | 0.418 |
| 37 | 1.061 |
| 38 | 1.915 |
| 39 | 0.502 |
| 40 | 1.331 |
| 41 | 2.061 |
| 42 | 1.477 |
| 43 | 2.444 |
| 44 | 0.378 |
| 45 | 2.493 |
| 46 | 1.422 |
| 47 | 1.811 |
| 48 | 1.063 |
| 49 | 1.793 |
| 50 | 1.791 |
| 51 | 0.735 |
| 52 | 0.591 |
| 53 | 1.231 |
| 54 | 0.346 |
| 55 | 2.423 |
| 56 | 0.372 |
| 57 | 2.169 |
| 58 | 1.378 |
| 59 | 0.441 |
| 60 | 0.521 |
| 61 | 0.710 |
| 62 | 0.376 |
| 63 | 1.427 |
| 64 | 0.404 |
| 65 | 0.514 |
| 66 | 2.098 |
| 67 | 0.293 |
| 68 | 0.663 |
| 69 | 1.823 |
| 70 | 0.786 |
| 71 | 0.397 |
| 72 | 2.675 |
| 73 | 0.706 |

### Example B-2

The GYS1 cell based assay is a bioluminescent assay that quantifies the glucose resulting from glycogen digestion; the quantified glucose is an indirect measure of GYS1 glycogen synthesis. Newly synthesized glycogen is digested using Glucoamylase; the resulting glucose is quantified by using the Glucose-glo assay kit from Promega. Glucose-glo works by coupling glucose oxidation and NADH production with a bioluminescent system that is activated with NADH. Glucose is oxidized by Glucose dehydrogenase and the reaction reduces NAD+ to NADH; NADH activates Reductase which reduces a pro-luciferin Reductase Substrate to luciferin. Luciferin is detected in a luciferase reaction using Ultra-Glo rLuciferase and ATP, and the luminesce produced is proportional to the glucose in the sample. The luminescence is measured as a single point read in a plate reader.

Compounds that inhibit the hGYS1 enzyme and, subsequently, the glycogen synthesis in cells, were tested using assay ready plates (white, clear bottom 384 well plates) in a final DMSO reaction volume of 1% DMSO. Compounds in the assay ready plates were mixed with media with no additives, except for 20 mM glucose prior to cell addition. HeLa cells were starved in media with no additives, except for 1× Glutamax for 24 h. Starved HeLa cells were plated, in a 1:1 ratio to the media in the assay ready plate and incubated for 24 h at 37° C. and 5% CO₂. Cells were washed in 1×PBS buffer and lysed in lysis buffer containing 50% 1×PBS and 25% 0.3 N HCl of the final volume in the well or reaction volume; cells were incubated with lysis buffer for 10 minutes and quenched with the remaining 25% of the reaction volume that consisted of 450 mM Tris pH 8.0. Lysates were mixed in a 1:1 ratio with Glucoamylase in 100 mM Sodium Acetate buffer, pH 5.3; the mixture was incubated for 1 h at 37° C. The digested lysate was mixed in a 1:1 ratio with Glucose-glo detection mixture as per vendor recommendations (Luciferase detection buffer, Reductase, Reductase substrate, Glucose dehydrogenase, and NAD) in read-out plates (solid white 384-well plates) and incubated for 1 h at RT. The plates were read using a plate reader with luminescence capabilities. Each compound concentration Relative Luminescence Unit (RLU) was averaged and normalized to the average RLU of the positive and negative controls to obtain a percentage inhibition. The normalized data vs. concentration was plotted; to determine the half-maximal concentration (IC₅₀), the Levenberg-Marquardt algorithm was used to fit a Hill equation to the dose response data.

The results are shown in Table 4 below, which reports the IC₅₀ of each compound. Unless otherwise specified, IC₅₀ values are reported as the geometric mean of at least 2 assay runs on separate days. As shown in the table below, the compounds of the present invention are potent inhibitors of human GYS1. Unless otherwise specified, IC₅₀ values are reported as the geometric mean of at least two assay runs on separate days. Each run represents the average of a technical replicate, where each compound was assayed twice in the same plate.

TABLE 4

| | Cellular |
|---|---|
| Cmpd | IC₅₀ |
| No. | (μM) |
| 2 | 6.52 |
| 3 | 1.51 |
| 4 | 2.79 |
| 5 | 1.06 |
| 11 | 3.28 |

### Example B-3

The GYS2 coupled enzyme assay is a kinetic biochemical assay that indirectly quantifies the rate of glycogen synthesis by coupling the conversion of GYS2 substrate UDP-glucose into UDP with downstream enzymatic reactions. UDP is released from UDP-glucose as glucose monomers are linked into the growing glycogen strand by GYS2. The coupled assay then proceeds with pyruvate kinase utilizing UDP and phospho(enol)pyruvate (PEP) to form pyruvate. Lactate dehydrogenase then converts pyruvate and NADH into lactate and NAD+. Oxidation of NADH to NAD+ can be measured continuously with a plate reader by quantifying the decrease in NADH absorbance at 340 nm over time.

Compounds that inhibit the hGYS2 enzyme and, subsequently, the downstream conversion of NADH to NAD+, were tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP 1 mM were added before splitting buffer into hGYS2 buffer and substrate buffer. To the hGYS2 buffer, rabbit liver glycogen was added at a final concentration of 0.2% glycogen. Glucose-6-Phosphate was added at 2 mM, recombinant hGYS2/GN1 protein was added at 200 nM to the substrate buffer, phosphoenolpyruvate (PEP) was added at 2 mM, UDP-Glucose was added at 2 mM, NADH was added at 0.6 mM, and Pyruvate Kinase/Lactate Dehydrogenase was added at 20 units/mL. The reaction was initiated by mixing hGYS2 buffer and substrate buffer at a 1:1 ratio. Both buffers were plated using a liquid dispensing device with hGYS2 buffer plated first followed by the substrate buffer. Plates were spun briefly to eliminate air bubbles and are immediately read in continuous mode at an absorbance of 340 nm, for 10 time points in one-minute increments, for a total of 10 minutes. The slope from these 10 time points was normalized to the positive and negative control wells. The duplicate % inhibition values are then averaged and fit to a Hill equation for dose response according to the Levenberg-Marquardt algorithm with the Hill equation maximum set to 100 and the minimum set to 0.

The results are shown in Table 5 below, which reports the IC₅₀ of each compound. Unless otherwise specified, IC₅₀ values are reported as the geometric mean of at least 2 assay runs on separate days. As shown in the table below, the compounds of the present invention are not potent inhibitors of human GYS2. Unless otherwise specified, IC₅₀ values are reported as the geometric mean of at least two assay runs on separate days. Each run represents the average of a technical replicate, where each compound was assayed twice in the same plate.

TABLE 5

| | GYS2 |
|---|---|
| | PK/LDH |
| Cmpd | IC₅₀ |
| No. | (μM) |
| 1 | >100.0 |
| 9 | >100.0 |
| 10 | >100.0 |
| 11 | >100.0 |
| 13 | >100.0 |
| 14 | >100.0 |
| 15 | >100.0 |
| 18 | >100.0 |
| 19 | >100.0 |
| 26 | >100.0 |
| 27 | >100.0 |
| 29 | >100.0 |
| 36 | >100.0 |
| 37 | >100.0 |
| 39 | >100.0 |
| 44 | >100.0 |
| 46 | >100.0 |
| 48 | >100.0 |
| 51 | >100.0 |
| 52 | >100.0 |
| 53 | >100.0 |
| 54 | >100.0 |
| 56 | >100.0 |
| 57 | >100.0 |
| 58 | >100.0 |
| 59 | >100.0 |
| 60 | >100.0 |
| 61 | >100.0 |
| 62 | >100.0 |
| 63 | >100.0 |
| 64 | >100.0 |
| 65 | >100.0 |
| 66 | >100.0 |
| 67 | >100.0 |
| 68 | >97.44 |
| 69 | >100.0 |
| 70 | >100.0 |
| 71 | >100.0 |
| 72 | >100.0 |
| 73 | >100.0 |

### Example B-4

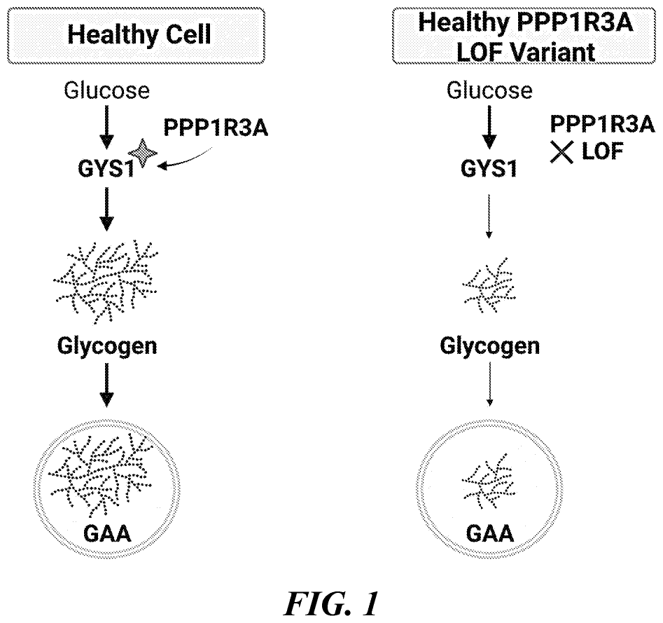

Pompe disease is a glycogen storage disease caused by mutations in the enzyme acid alpha-glucosidase resulting in pathological accumulation of glycogen. Glycogen can accumulate in virtually all tissues, but the primary pathology affects skeletal and cardiac muscle. Inhibiting the synthesis of muscle glycogen could reduce the pathologic build-up of glycogen by acting as a substrate reduction therapy. Savage et. al. identified a predicted protein truncating variant (PTV) in the PPP1R3A gene (a regulator of glycogen metabolism) in ˜0.5% of Europeans, which results in ˜65% reduction in muscle glycogen (Savage et. al., A Prevalent Variant in PPP1R3A Impairs Glycogen Synthesis and Reduces Muscle Glycogen Content in Humans and Mice. PLoS Medicine. 2008; herein incorporated by reference in its entirety). PPP1R3A functions as a key activator of muscle glycogen synthase 1 (GYS1) by dephosphorylating the enzyme and maximizing activity. FIG. 1 demonstrates the pathway in which PPP1R3A (loss of function) LoF leads to reduction in muscle glycogen.

Large biobanks enable investigation of the consequences of genetic variation on many health-related phenotypes. To assess the consequences of a predicted 65% loss of muscle glycogen, association study was performed in the UK Biobank comparing phenotypes between PPP1R3A PTV carriers and non-carriers. Genetic association studies were performed using REGENIE (Mbatchou, J., Barnard, L., Backman, J. et al. Computationally efficient whole-genome regression for quantitative and binary traits. Nat Genet 53, 1097-1103, 2021), adjusted for age, sex, and the first 10 principal components of ancestry. Quantitative traits were normalized using an inverse rank normal transformation.

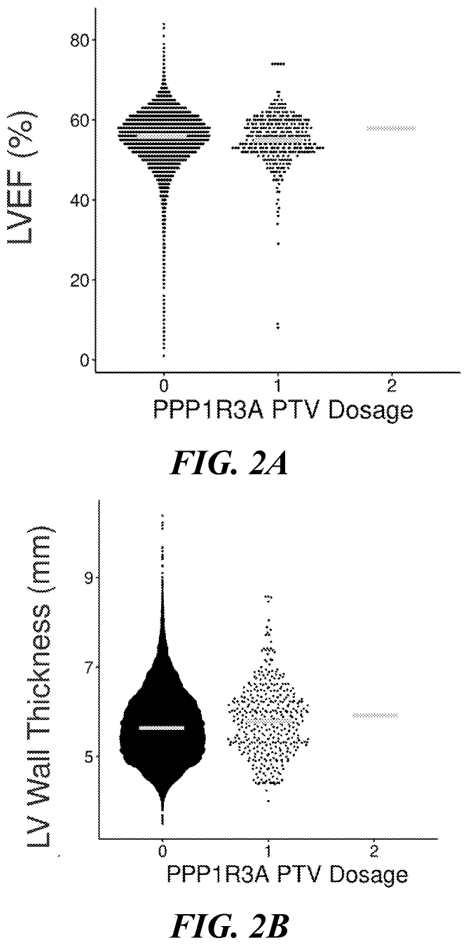

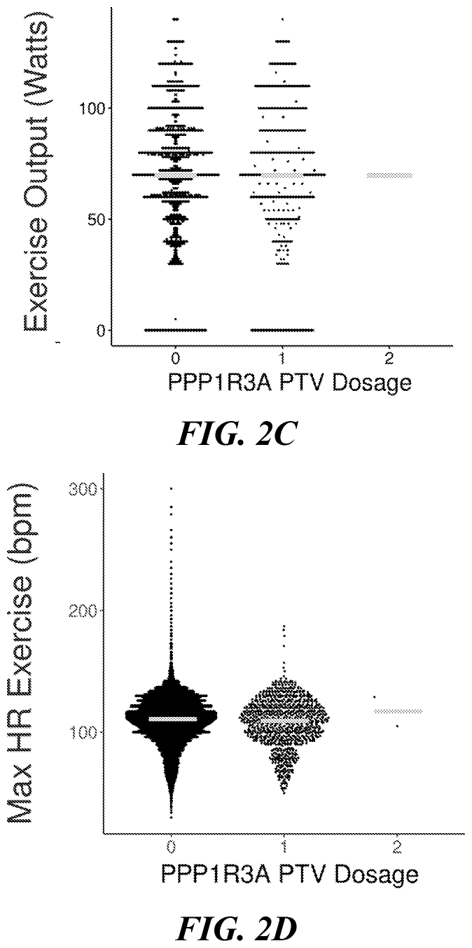

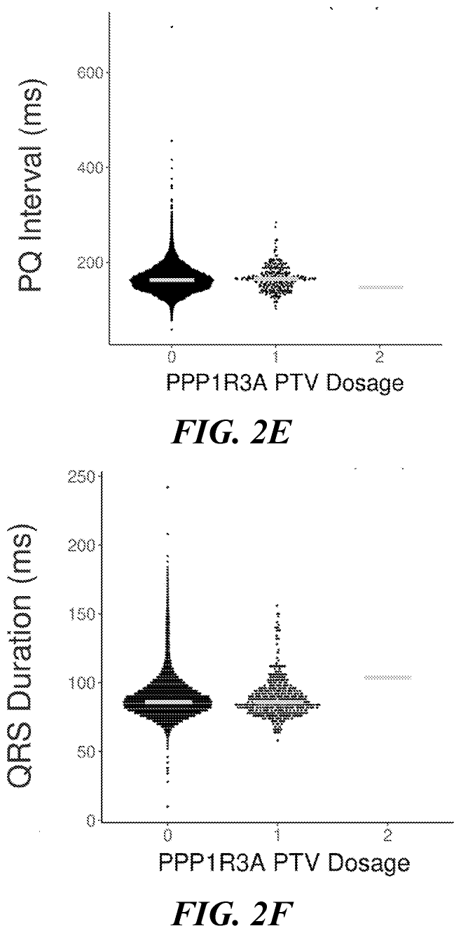

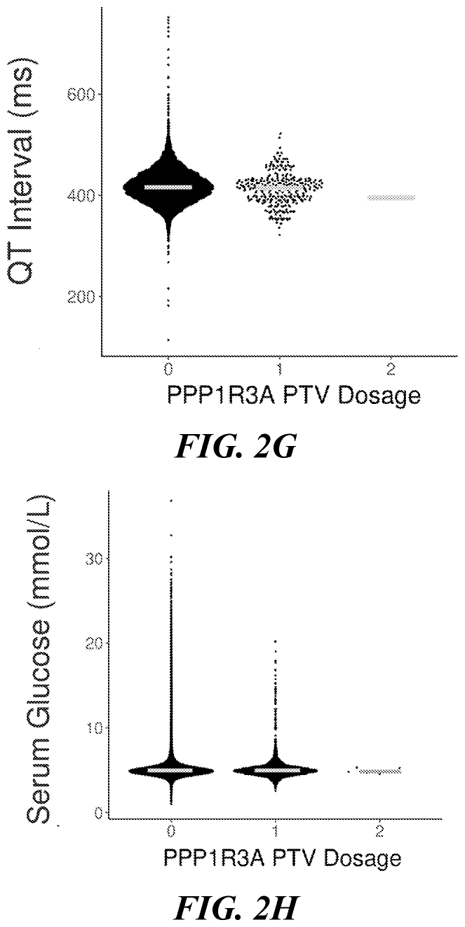

With regards to FIGS. 2A-2H, the association between PPP1R3A PTV and the quantitative phenotypes of left ventricular ejection (LVEF) (%) (FIG. 2A), left ventricle wall thickness (mm) (FIG. 2B), exercise output (watts) (FIG. 2C), max heart rate (HR) exercise (bpm) (FIG. 2D), PQ interval (ms) (FIG. 2E), QRS duration (ms) (FIG. 2F), QT interval (ms) (FIG. 2G), and serum glucose (mmol/L) (FIG. 2H), are depicted. Phenotype values are plotted by PPP1R3A dosage for UK Biobank participants. No association between PPP1R3A PTV and the quantitative phenotypes in the UK Biobank was identified.

Table 6 below lists the P-value and number of participants (N) for the results depicted in FIGS. 2A-H. No associations between PPP1R3A PTV and cardiac parameters, including left ventricular ejection fraction (p=0.871) and wall thickness (p=0.168) were identified. There was no evidence of changes in EKG cardiac conduction intervals nor in any muscle performance measurements (n=49,616), including maximum heart rate (p=0.444) and maximum workload during an exercise test (p=0.100). Further, no changes in serum glucose (p=0.71) or any other members of a panel of ˜170 serum metabolites were observed.

TABLE 6

| Phenotype | P-value | N |
|---|---|---|
| LVEF | 0.871 | 27,716 |
| LV Wall Thickness | 0.168 | 27,579 |
| Exercise Output | 0.100 | 49,616 |
| Max HR Exercise | 0.444 | 49,603 |
| QRS Duration | 0.527 | 29,507 |
| PQ Interval | 0.366 | 16,694 |
| QT Interval | 0.222 | 17,574 |
| Serum Glucose | 0.477 | 294,042 |

As shown in Table 7 below, no association between PPP1R3A PTV and key health outcomes was also observed. In addition to the phenotypes in Table 7, no phenome-wide significant associations between PPP1R3A PTV and rates of any ICD10 code with over 100 occurrences in UK Biobank was observed.

TABLE 7

| Disease | Effect (SE) | P-value | N Cases |
|---|---|---|---|
| Type 2 Diabetes | −0.094 (0.073) | 0.200 | 18,868 |
| Liver Cirrhosis | −0.041 (0.273) | 0.880 | 1,325 |
| Heart Failure | −0.061 (0.127) | 0.630 | 6,117 |

After performing an extensive Phenome-wide association study in UK Biobank, no significant associations between any key outcomes or phenotypes and loss of function of PPP1R3A were found. The results provided herein demonstrate that loss of function variants in the PPP1R3A gene are not associated with adverse health outcomes in a large biobank population. This suggests that partial reduction in muscle glycogen (˜65%) from birth is well tolerated and supports the potential safety of pharmacologic reduction of muscle glycogen.

All publications, patent applications, patents, and other references mentioned herein are expressly incorporated by reference in their entireties, to the same extent as if each were incorporated by reference individually.

It is to be understood that, while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula (I): 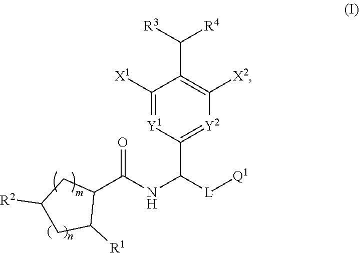
or a stereoisomer or tautomer thereof, or a pharmaceutically acceptable salt of any of the foregoing, wherein:
Y¹ and Y² are each CH, or
one of Y¹ and Y² is N and the other of Y¹ and Y² is CH;
X¹ and X² are each independently H or halo;
R³ and R⁴ are each —CH₃, or
R³ and R⁴ are taken, together with the atoms to which they are attached, to form cyclopropyl or cyclobutyl;
either
(1) L is absent; and
Q¹ is:
(i) C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl, or
(ii) 3-15 membered heterocyclyl, wherein the 3-15 membered heterocyclyl of Q¹ is optionally substituted with one or more oxo, or
(iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl;
or
(2) L is —CH₂—; and
Q¹ is C₃₋₁₀cycloalkyl;
m is 0 or 1;
n is 0 or 1;
R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein
the C₁₋₆alkyl of the —C(O)—NH(C₁₋₆alkyl) of R¹ is optionally substituted with one or more —C(O)—C₁₋₆alkoxy, and
the C₁₋₆alkyl of the —NH—C(O)—C₁₋₆alkyl of R¹ is optionally substituted with one or more —NH—C(O)—C₁₋₆alkyl or —C(O)—NH₂; and
R² is H, halo, or —OH.

2. The compound of claim 1, or a stereoisomer or tautomer thereof, or a pharmaceutically acceptable salt of any of the foregoing, wherein the compounds of formula (I) has a stereochemical configuration of

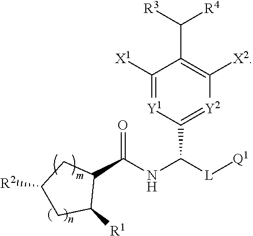

3. The compound of claim 1, or a stereoisomer or tautomer thereof, or a pharmaceutically acceptable salt of any of the foregoing, wherein X¹ is H.

4. The compound of claim 1, or a stereoisomer or tautomer thereof, or a pharmaceutically acceptable salt of any of the foregoing, wherein X¹ is halo.

5. The compound of claim 1 or claim 4, or a stereoisomer or tautomer thereof, or a pharmaceutically acceptable salt of any of the foregoing, wherein X¹ is fluoro.

6. The compound of any one of claims 1-5, or a stereoisomer or tautomer thereof, or a pharmaceutically acceptable salt of any of the foregoing, wherein X² is H.

7. The compound of any one of claims 1-5, or a stereoisomer or tautomer thereof, or a pharmaceutically acceptable salt of any of the foregoing, wherein X² is halo.

8. The compound of any one of claims 1-5 and 7, or a stereoisomer or tautomer thereof, or a pharmaceutically acceptable salt of any of the foregoing, wherein X² is fluoro.

9. The compound of any one of claims 1-8 or a stereoisomer or tautomer thereof, or a pharmaceutically acceptable salt of any of the foregoing, wherein R³ and R⁴ are each independently —CH₃.

10. The compound of any one of claims 1-8 or a stereoisomer or tautomer thereof, or a pharmaceutically acceptable salt of any of the foregoing, wherein R³ and R⁴ are taken, together with the atoms to which they are attached, to form cyclopropyl or cyclobutyl.

11. The compound of any one of claims 1-8 and 10, or a stereoisomer or tautomer thereof, or a pharmaceutically acceptable salt of any of the foregoing, wherein R³ and R⁴ are taken, together with the atoms to which they are attached, to form cyclopropyl.

12. The compound of any one of claims 1-8 and 10, or a stereoisomer or tautomer thereof, or a pharmaceutically acceptable salt of any of the foregoing, wherein R³ and R⁴ are taken, together with the atoms to which they are attached, to form cyclobutyl.

13. The compound of any one of claims 1-12, or a stereoisomer or tautomer thereof, or a pharmaceutically acceptable salt of any of the foregoing, wherein L is absent.

14. The compound of any one of claims 1-13, or a stereoisomer or tautomer thereof, or a pharmaceutically acceptable salt of any of the foregoing, wherein Q¹ is C₆₋₂₀aryl, wherein the C₆₋₂₀aryl of Q¹ is optionally substituted with one or more NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), or —NH—C(═N—CN)—NH₂, —NH(C₁₋₆alkyl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

15. The compound of any one of claims 1-14, or a stereoisomer or tautomer thereof, or a pharmaceutically acceptable salt of any of the foregoing, wherein Q¹ is phenyl, wherein the phenyl of Q¹ is optionally substituted with one or more NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), or —NH—C(═N—CN)—NH₂, —NH(C₁₋₆alkyl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl.

16. The compound of any one of claims 1-15, or a stereoisomer or tautomer thereof, or a pharmaceutically acceptable salt of any of the foregoing, wherein Q¹ is selected from the group consisting of

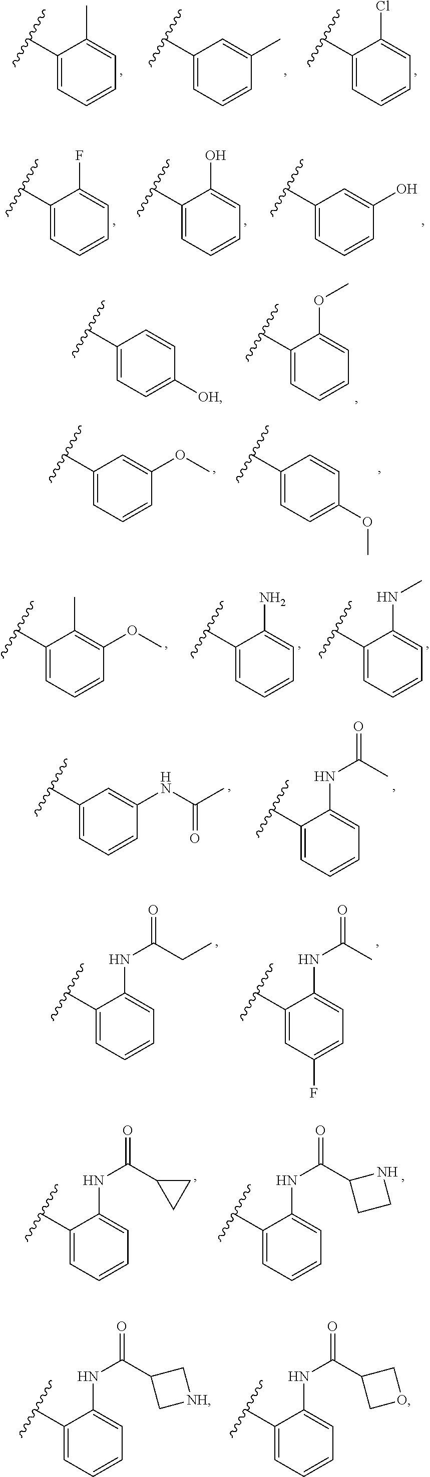

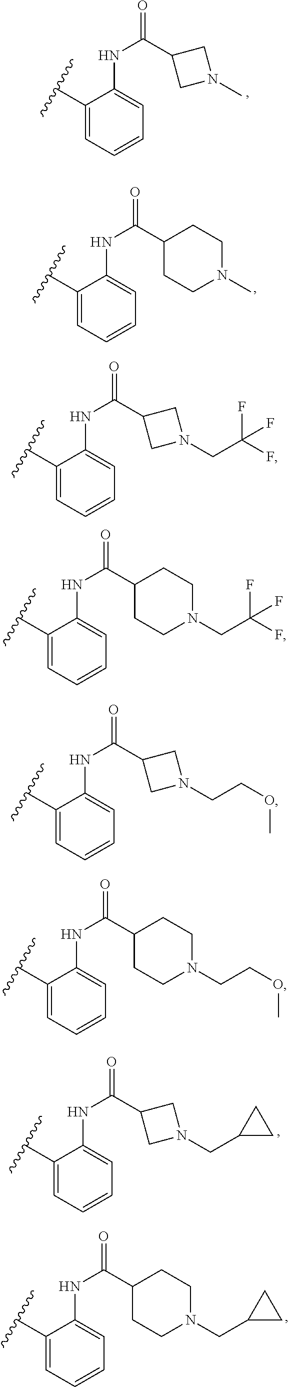

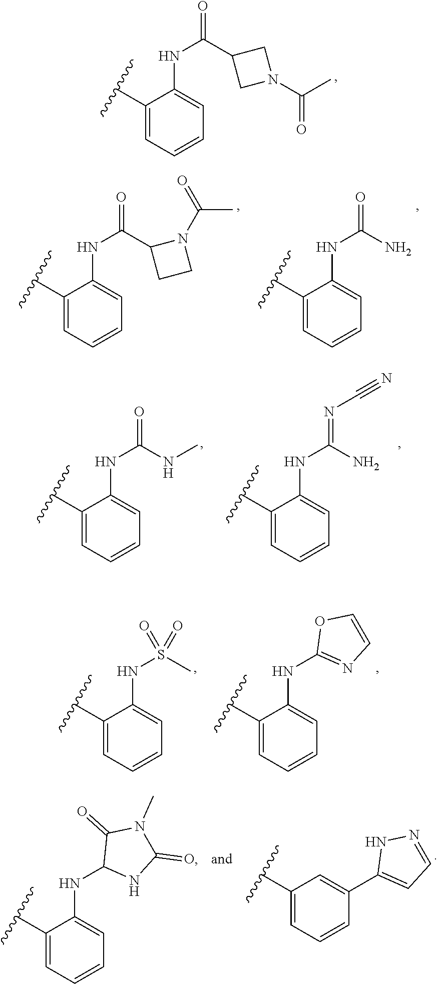

17. The compound of any one of claims 1-13, or a stereoisomer or tautomer thereof, or a pharmaceutically acceptable salt of any of the foregoing, wherein Q¹ is 3-15 membered heterocyclyl, wherein the 3-15 membered heterocyclyl of Q¹ is optionally substituted with one or more oxo.

18. The compound of any one of claims 1-13 and 17, or a stereoisomer or tautomer thereof, or a pharmaceutically acceptable salt of any of the foregoing, wherein Q¹ is 9-10 membered heterocyclyl, wherein the 9-10 membered heterocyclyl of Q¹ is optionally substituted with one or more oxo.

19. The compound of any one of claims 1-13, 17, and 18, or a stereoisomer or tautomer thereof, or a pharmaceutically acceptable salt of any of the foregoing, wherein Q¹ is selected from the group consisting of

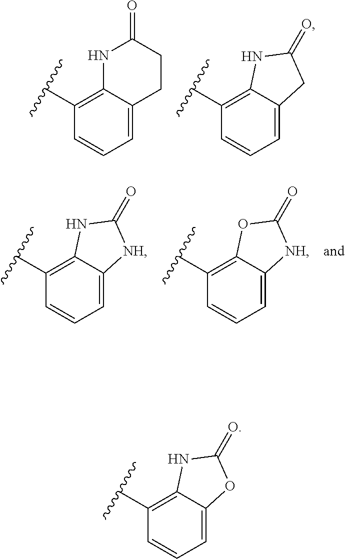

20. The compound of any one of claims 1-13, or a stereoisomer or tautomer thereof, or a pharmaceutically acceptable salt of any of the foregoing, wherein Q¹ is 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl.

21. The compound of any one of claims 1-13 and 20, or a stereoisomer or tautomer thereof, or a pharmaceutically acceptable salt of any of the foregoing, wherein Q¹ is 6-10 membered heteroaryl, wherein the 6-10 membered heteroaryl of Q¹ comprises at least one annular N atom and is optionally substituted with one or more —NH₂, halo, C₁₋₆alkyl, or C₃₋₁₀cycloalkyl.

22. The compound of any one of claims 1-13, 20, and 21, or a stereoisomer or tautomer thereof, or a pharmaceutically acceptable salt of any of the foregoing, wherein Q¹ is selected from the group consisting of

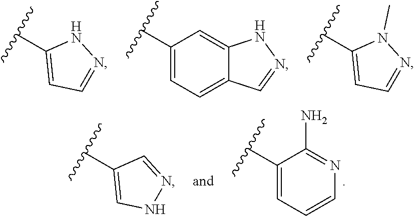

23. The compound of any one of claims 1-12, or a stereoisomer or tautomer thereof, or a pharmaceutically acceptable salt of any of the foregoing, wherein L is —CH₂— and Q¹ is C₃₋₁₀cycloalkyl.

24. The compound of claim any one of claims 1-23, or a stereoisomer or tautomer thereof, or a pharmaceutically acceptable salt of any of the foregoing, wherein m and n are each independently 0.

25. The compound of any one of claims 1-23, or a stereoisomer or tautomer thereof, or a pharmaceutically acceptable salt of any of the foregoing, wherein m and n are each independently 1.

26. The compound of any one of claims 1-25, or a stereoisomer or tautomer thereof, or a pharmaceutically acceptable salt of any of the foregoing, wherein R¹ is selected from the group consisting of H, —CN, —C(O)—NH₂, —C(O)—NH(CN), —C(O)—NH(C₁₋₆alkyl), —NH—C(O)—NH₂, and —NH—C(O)—C₁₋₆alkyl, wherein
the C₁₋₆alkyl of the —C(O)—NH(C₁₋₆alkyl) is optionally substituted with one or more —C(O)—C₁₋₆alkoxy, and
the C₁₋₆alkyl of the —NH—C(O)—C₁₋₆alkyl is optionally substituted with one or more —NH—C(O)—C₁₋₆alkyl or —C(O)—NH₂.

27. The compound of any one of claims 1-26, or a stereoisomer or tautomer thereof, or a pharmaceutically acceptable salt of any of the foregoing, wherein R¹ is H.

28. The compound of any one of claims 1-26, or a stereoisomer or tautomer thereof, or a pharmaceutically acceptable salt of any of the foregoing, wherein R¹ is selected from the group consisting of —CN, —C(O)—NH₂, —C(O)—NH(CN), —C(O)—NH(C₁₋₆alkyl), —NH—C(O)—NH₂, and —NH—C(O)—C₁₋₆alkyl, wherein
the C₁₋₆alkyl of the —C(O)—NH(C₁₋₆alkyl) is optionally substituted with one or more —C(O)—C₁₋₆alkoxy, and
the C₁₋₆alkyl of the —NH—C(O)—C₁₋₆alkyl is optionally substituted with one or more —NH—C(O)—C₁₋₆alkyl or —C(O)—NH₂.

29. The compound of any one of claims 1-26 and 28, or a stereoisomer or tautomer thereof, or a pharmaceutically acceptable salt of any of the foregoing, wherein R¹ is selected from the group consisting of —CN,

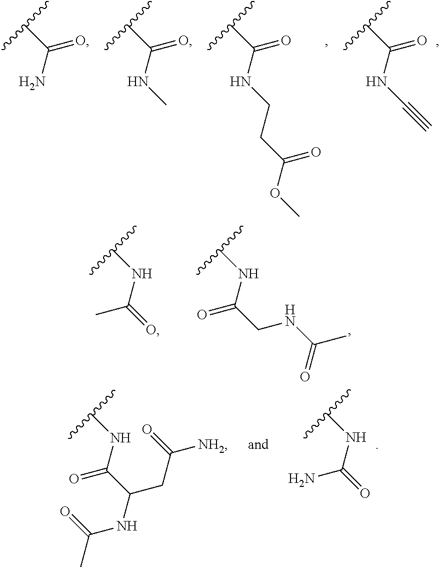

30. The compound of any one of claims 1-25, or a stereoisomer or tautomer thereof, or a pharmaceutically acceptable salt of any of the foregoing, wherein R¹ is H or halo.

31. The compound of any one of claims 1-25 and 30, or a stereoisomer or tautomer thereof, or a pharmaceutically acceptable salt of any of the foregoing, wherein R¹ is H or fluoro.

32. The compound of any one of claims 1-25, 30, and 31, or a stereoisomer or tautomer thereof, or a pharmaceutically acceptable salt of any of the foregoing, wherein R¹ is fluoro.

33. The compound of any one of claims 1-32, or a stereoisomer or tautomer thereof, or a pharmaceutically acceptable salt of any of the foregoing, wherein R² is H.

34. The compound of any one of claims 1-32, or a stereoisomer or tautomer thereof, or a pharmaceutically acceptable salt of any of the foregoing, wherein R² is halo.

35. The compound of any one of claims 1-32, and 34, or a stereoisomer or tautomer thereof, or a pharmaceutically acceptable salt of any of the foregoing, wherein R² is fluoro.

36. The compound of any one of claims 1-32, or a stereoisomer or tautomer thereof, or a pharmaceutically acceptable salt of any of the foregoing, wherein R² is —OH.

37. The compound of any one of claims 1-21, and 24-36, or a stereoisomer or tautomer thereof, or a pharmaceutically acceptable salt of any of the foregoing, wherein the compound is of formula (I-A): 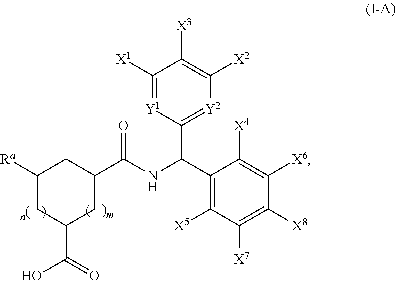
or a stereoisomer or tautomer thereof, or a pharmaceutically acceptable salt of any of the foregoing, wherein
either:
i. X⁴⁻⁸ are each independently H, —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)2-C₁₋₆alkyl, —NH(C₁₋₆alkyl), —NH-(3-15 membered heterocyclyl), or —NH-(5-20 membered heteroaryl), wherein
the 3-9 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-9 membered heterocyclyl of the —NH-(3-15 membered heterocyclyl) is optionally substituted with one or more oxo or C₁₋₆alkyl; or
ii. X⁶ is taken together with either of X⁴ or X⁸, and the atoms to which they are attached, to form ring A, wherein ring A is
3-9 membered heterocyclyl, and wherein the 3-9 membered heterocyclyl of ring A is optionally substituted with one or more oxo, wherein X⁵, X⁷, and the other of X⁴ or X⁸ are each independently H, or oxo, or
5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl, wherein X⁵, X⁷, and the other of X⁴ or X⁸ are each independently H, —NH₂, halo, C₁₋₆alkyl, or C₃₋₁₀cycloalkyl; or
iii. X⁷ is taken together with either of X⁵ or X⁸, and the atoms to which they are attached, to form ring A, wherein ring A is
3-9 membered heterocyclyl, and wherein the 3-9 membered heterocyclyl of ring A is optionally substituted with one or more oxo, and wherein X⁴, X⁶, and the other of X⁵ or X⁸ are each independently H, or oxo, or 5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl, and wherein X⁴, X⁶, and the other of X⁵ or X⁸ are
each independently H, —NH₂, halo, C₁₋₆alkyl, or C₃₋₁₀cycloalkyl.

38. The compound of any one of claims 1-10, 13, 15, or 20-23, or a stereoisomer or tautomer thereof, or a pharmaceutically acceptable salt of any of the foregoing, wherein the compound is of formula (I-C): 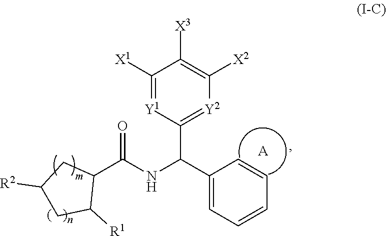
or a stereoisomer or tautomer thereof, or a pharmaceutically acceptable salt of any of the foregoing, wherein ring A is
3-9 membered heterocyclyl, and wherein the 3-9 membered heterocyclyl of ring A is optionally substituted with one or more oxo,
5-14 membered heteroaryl, and wherein the 5-14 membered heteroaryl of ring A comprises at least one annular N atom and is optionally substituted with one or more —NH₂, halo, C₁₋₆alkyl, or C₃₋₁₀cycloalkyl.

39. The compound of claim 1, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from Table 1.

40. A process for preparing a compound of any one of claims 1-39, or a stereoisomer or tautomer thereof, or a pharmaceutically acceptable salt of any of the foregoing, wherein the process comprises:
(a) reacting a compound of formula (I-1): 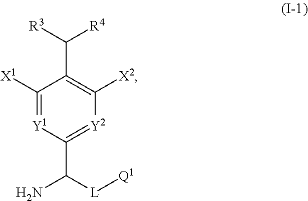
or a salt thereof, wherein
Y¹ and Y² are each CH, or
one of Y¹ and Y² is N and the other of Y¹ and Y² is CH;
X¹ and X² are each independently H or halo;
R³ and R⁴ are each —CH₃, or
R³ and R⁴ are taken, together with the atoms to which they are attached, to form cyclopropyl or cyclobutyl;
either
(1) L is absent; and
Q¹ is:
(i) C₆₋₂₀aryl, wherein the C₆₋₂₀aryl of Q¹ is optionally substituted with one or more —OH, —NH₂, halo, C₁₋₆alkyl, C₁₋₆alkoxy, C₃₋₁₀cycloalkyl, 5-20 membered heteroaryl, —NH—C(O)—NH₂, —NH—C(O)—NH(C₁₋₆alkyl), —NH—C(O)—C₁₋₆alkyl, —NH—C(O)—C₃₋₁₀cycloalkyl, —NH—C(O)-(3-15 membered heterocyclyl), —NH—C(═N—CN)—NH₂, —NH—S(O)₂—C₁₋₆alkyl, —NH(C₁₋₆alkyl), —NH-(3-15 membered heterocyclyl), or —NH-(5-20 membered heteroaryl), wherein
the 3-15 membered heterocyclyl of the —NH—C(O)-(3-15 membered heterocyclyl) is optionally substituted with one or more —C(O)—C₁₋₆alkyl or C₁₋₆alkyl, wherein the C₁₋₆alkyl is optionally substituted with one or more halo, C₁₋₆alkoxy, or C₃₋₁₀cycloalkyl, and
the 3-15 membered heterocyclyl of the —NH-(3-15 membered heterocyclyl) is optionally substituted with one or more oxo or C₁₋₆alkyl, or
(ii) 3-15 membered heterocyclyl, wherein the 3-15 membered heterocyclyl of Q¹ is optionally substituted with one or more oxo, or
(iii) 5-20 membered heteroaryl, wherein the 5-20 membered heteroaryl of Q¹ comprises at least one annular N atom and is optionally substituted with one or more —NH₂, halo, C₁₋₆alkyl, or C₃₋₁₀cycloalkyl;
or
(2) L is —CH₂—; and
Q¹ is C₃₋₁₀cycloalkyl,
with a compound of formula (I-2): 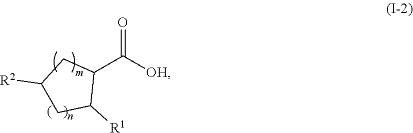
wherein,
m is 0 or 1;
n is 0 or 1;
R¹ is H, halo, —CN, —C(O)—NH₂, —C(O)—NH(CN), —C(O)—NH(C₁₋₆alkyl), —NH—C(O)—NH₂, or —NH—C(O)—C₁₋₆alkyl, wherein
the C₁₋₆alkyl of the —C(O)—NH(C₁₋₆alkyl) of R¹ is optionally substituted with one or more —C(O)—C₁₋₆alkoxy, and
the C₁₋₆alkyl of the —NH—C(O)—C₁₋₆alkyl of R¹ is optionally substituted with one or more —NH—C(O)—C₁₋₆alkyl or —C(O)—NH₂; and
R² is H, halo, or —OH
in the presence of a coupling reagent to provide a compound of formula (I).

41. A pharmaceutical composition comprising (i) a compound of any one of claims 1-39, or a stereoisomer or tautomer thereof, or a pharmaceutically acceptable salt of any of the foregoing, and (ii) one or more pharmaceutically acceptable excipients.

42. A method of treating a GYS1-mediated disease, disorder, or condition in an individual in need thereof, comprising administering to the individual an effective amount of a compound of any one of claims 1-39, or a stereoisomer or tautomer thereof, or a pharmaceutically acceptable salt of any of the foregoing, or a pharmaceutical composition of claim 41.

43. The method of claim 42, wherein the disease, disorder, or condition is a glycogen storage disorder (GSD).

44. The method of claim 42 or claim 43, wherein the disease, disorder, or condition is selected from the group consisting of Pompe disease, Cori disease (GSD III), adult polyglucosan body disease (APBD), and Lafora disease.

45. The method of any one of claims 42-44, wherein the disease, disorder, or condition is Pompe disease.

46. The method of claim 42, wherein the disease, disorder, or condition is cancer.

47. The method of claim 42 or claim 46, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

48. The method of claim 42, wherein the individual has a GAA mutation.

49. The method of claim 48, wherein the GAA mutation is a loss-of-function mutation.

50. A kit, comprising (i) a compound of any one of claims 1-39, or a stereoisomer or tautomer thereof, or a pharmaceutically acceptable salt of any of the foregoing, or a pharmaceutical composition of claim 41, and (ii) instructions for use in treating an GYS1-mediated disease, disorder, or condition in an individual in need thereof.

51. The kit of claim 50, wherein the disease, disorder, or condition is a glycogen storage disorder (GSD).

52. The kit of claim 50 or claim 51, wherein the disease, disorder, or condition is selected from the group consisting of Pompe disease, Cori disease (GSD III), adult polyglucosan body disease (APBD), and Lafora disease.

53. The kit of any one of claims 50-52, wherein the disease, disorder, or condition is Pompe disease.

54. The kit of claim 50, wherein the disease, disorder, or condition is cancer.

55. The kit of claim 50 or claim 54, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

56. The kit of claim 52, wherein the individual has a GAA mutation.

57. The kit of claim 56, wherein the GAA mutation is a loss-of-function mutation.

58. A method of modulating GYS1 in a cell, comprising exposing the cell to a composition comprising an effective amount of a compound of any one or claims 1-39, or a stereoisomer or tautomer thereof, or a pharmaceutically acceptable salt of any of the foregoing, or a pharmaceutical composition of claim 41.

59. A method of inhibiting GYS1 in a cell, comprising exposing the cell to a composition comprising an effective amount of a compound of any one or claims 1-39, or a stereoisomer or tautomer thereof, or a pharmaceutically acceptable salt of any of the foregoing, or a pharmaceutical composition of claim 41.

60. A method of reducing tissue glycogen stores in an individual in need thereof, comprising administering to the individual an effective amount of a compound of any one of claims 1-39, or a stereoisomer or tautomer thereof, or a pharmaceutically acceptable salt of any of the foregoing, or a pharmaceutical composition of claim 41.

61. A method of treating a GYS1-mediated disease, disorder, or condition in an individual in need thereof, comprising subjecting the individual to glycogen substrate reduction therapy, wherein the glycogen substrate reduction therapy comprises administering to the individual an effective amount of a compound of any one of claims 1-39, or a stereoisomer or tautomer thereof, or a pharmaceutically acceptable salt of any of the foregoing, or the pharmaceutical composition of claim 39.

62. The method of claim 61, comprising subjecting the individual to glycogen substrate reduction therapy in combination with enzyme replacement therapy.

63. The method of claim 62, wherein the enzyme replacement therapy is selected from the group consisting of alglucosidase alfa (human recombinant alpha-glucosidase (human GAA)) Myozyme and Lumizyme.

64. The method of any one of claims 61-63, wherein the disease, disorder, or condition is a glycogen storage disorder (GSD).

65. The method of any one of claims 61-64, wherein the disease, disorder, or condition is selected from the group consisting of Pompe disease, Cori disease (GSD III), adult polyglucosan body disease (APBD), and Lafora disease.

66. The method of any one of claims 61-65, wherein the disease, disorder, or condition is Pompe disease.

67. The method of any one of claims 61-63, wherein the disease, disorder, or condition is cancer.

68. The method of any one of claims 61-63, or 67, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

69. The method of any one of claims 61-63, wherein the individual has a GAA mutation.

70. The method of claim 69, wherein the GAA mutation comprises a loss-of-function mutation.

71. The method of any one of claims 58-60 wherein the compound is selective for GYS1 over GYS2.

72. The method of claim 71, wherein the compound is greater than 500 or 1,000 or 1,500 or 1,700-fold selective for GYS1 over GYS2.

73. The method of any one of claims 42-49 or 58-72, comprising reducing the level of glycogen in skeletal muscle.

74. A compound of any one of claims 1-39, or a stereoisomer or tautomer thereof, or a pharmaceutically acceptable salt of any of the foregoing, or a pharmaceutical composition of claim 41, for use in treating a GYS1-mediated disease, disorder, or condition in an individual in need thereof.

75. A compound of any one of claims 1-39, or a stereoisomer or tautomer thereof, or a pharmaceutically acceptable salt of any of the foregoing, or a pharmaceutical composition of claim 41, for use in modulating GYS1 in a cell.

76. A compound of any one of claims 1-39, or a stereoisomer or tautomer thereof, or a pharmaceutically acceptable salt of any of the foregoing, or a pharmaceutical composition of claim 41, for use in inhibiting GYS1 in a cell.

77. A compound of any one of claims 1-39, or a stereoisomer or tautomer thereof, or a pharmaceutically acceptable salt of any of the foregoing, or a pharmaceutical composition of claim 41, for use in reducing tissue glycogen stores in an individual in need thereof.

78. A compound of any one of claims 1-39, or a stereoisomer or tautomer thereof, or a pharmaceutically acceptable salt of any of the foregoing, or the pharmaceutical composition of claim 41, for use in a glycogen substrate reduction therapy for treating a GYS1-mediated disease, disorder, or condition in an individual in need thereof.

79. Use of a compound of any one of claims 1-39, or a stereoisomer or tautomer thereof, or a pharmaceutically acceptable salt of any of the foregoing, or a pharmaceutical composition of claim 41, in the manufacture of a medicament for use in treating a GYS1-mediated disease, disorder, or condition in an individual in need thereof.

80. Use of a compound of any one of claims 1-39, or a stereoisomer or tautomer thereof, or a pharmaceutically acceptable salt of any of the foregoing, or a pharmaceutical composition of claim 41, in the manufacture of a medicament for use in modulating GYS1 in a cell.

81. Use of a compound of any one of claims 1-39, or a stereoisomer or tautomer thereof, or a pharmaceutically acceptable salt of any of the foregoing, or a pharmaceutical composition of claim 41, in the manufacture of a medicament for use in inhibiting GYS1 in a cell.

82. Use of a compound of any one of claims 1-39, or a stereoisomer or tautomer thereof, or a pharmaceutically acceptable salt of any of the foregoing, or a pharmaceutical composition of claim 41, in the manufacture of a medicament for use in reducing tissue glycogen stores in an individual in need thereof.

83. Use of a compound of any one of claims 1-39, or a stereoisomer or tautomer thereof, or a pharmaceutically acceptable salt of any of the foregoing, or the pharmaceutical composition of claim 41, in the manufacture of a medicament for use in a glycogen substrate reduction therapy for treating a GYS1-mediated disease, disorder, or condition in an individual in need thereof.
